(19) United States
(12) Patent Application Publication
(10) Pub. No.: US 2008/0248589 A1
(43) Pub. Date: Oct. 9, 2008

(54) Sample Presentation Device

(76) Inventors: Christopher M. Belisle, Concord, CA (US); John A. Walker, Hayward, CA (US); Sarah M. Ngola, Sunnyvale, CA (US); Douglas P. Greiner, Fremont, CA (US); Mark J. Levy, San Jose, CA (US); Xiaoxia Zhao, Fremont, CA (US); Irene Y. Chen, Fremont, CA (US); Mark L. Stolowitz, Pleasanton, CA (US); Donald P. Paquin, San Jose, CA (US)

Correspondence Address: MCDERMOTT WILL & EMERY LLP, 600 13TH STREET, N.W., WASHINGTON, DC, 20005-3096, US

(21) Appl. No.: 11/596,285

(22) PCT Filed: May 19, 2005
(86) PCT No.: PCT/US05/17813

§ 371 Date: Mar. 3, 2008

Related U.S. Application Data
(63) Continuation of application No. 11/036,707, filed on Jan. 13, 2005.
Application No. 11/036,707 is a continuation-in-part of application No. PCT/US03/21786, filed on Jul. 14, 2003.
(60) Provisional application No. 60/573,440, filed on May 21, 2004.
(60) Provisional application No. 60/573,440, filed on May 21, 2004.

(51) Int. Cl. G01N 33/543; B01J 19/00

(52) U.S. Cl. 436/518; 422/68.1

(57) ABSTRACT

The present invention relates to sample presentation devices useful in performing analytical measurements. These devices have been configured to enable various aspects of liquid handling such as: retention, storage, transport, concentration, positioning, and transfer. Additionally, these devices can enhance the detection and characterization of analytes. The sample presentation devices of the present invention are comprised of one or more substrates having a plurality of zones of differing wettability. Methods of analyzing samples using the sample presentation device of the invention, as well as methods of making the sample presentation devices are disclosed.

### CROSS-REFERENCE TO RELATED APPLICATION

This application is a continuation of U.S. application Ser. No. 11/036,707, entitled “SAMPLE PRESENTATION DEVICE” filed on Jan. 13, 2004, which claims the benefit of U.S. provisional application No. 60/573,440 entitled “SAMPLE PRESENTATION DEVICE” filed on May 21, 2004, each of which is incorporated herein by reference in its entirety.

### FIELD OF THE INVENTION

The present invention relates to sample presentation devices useful in performing analytical measurements. In addition, the present invention relates to the fabrication and use of sample presentation devices.

### BACKGROUND OF THE INVENTION

Most scientific fields that involve some kind of chemical and biological analysis of a sample require researchers to be able to identify and measure compounds or analytes found in aqueous solutions (e.g., the measurement of proteins in blood plasma or the measurement of pesticides in runoff from streams). Here, analytes generally refer to component(s) of a liquid sample that are of interest to an investigator. Typically, fluid samples containing analytes are presented to an analytical measurement instrument by means of a container (e.g., test tube, multiwell plate, or cuvette) or other presentation device (e.g., slide or biochip). Because of the overriding interest in measuring a large number of samples quickly (so called “high throughput” measurement of samples), much attention has been paid to developing standardized containers and devices that can be used in connection with automated analytical instruments. For example, in the drug discovery field, researchers interested in screening drug candidates frequently screen thousands or even millions of possible drug candidates using various analytical techniques (e.g., fluorescence polarization detection), many of which use standard 384 well plates to contain the sample solutions containing the drug candidates. As such, sample presentation devices constitute a critically important component of a researcher's analytical equipment in a wide range of scientific fields, ranging from genomics and proteomics, drug development, clinical diagnostics, and analysis of environmental or biological toxins or agents (e.g., assessing environmental contamination and screening for possible agents used in bioterrorism).

In genomics and proteomics, for example, the focus is on the identification and study of DNA/RNA and proteins/peptides, respectively. These fields collectively refer to the systemic study of chemical and biological moieties in living organisms, their interactions, and the analytical techniques required to discern them. Understanding complex living systems, rather than individual cell components, is a major focus of current biological and biomedical research in both fields. Specifically, a principal aim of genomics is to sequence and generate large databases of the gene content of entire organisms. Genomes have been compiled for bacteria, yeast, nematodes, drosophila, and, most recently, humans. Similarly, proteomics is the study of all proteins expressed at a specific time in the cell, a principal aim of which is to obtain partial protein amino acid sequences that can be used with database matching tools to identify an entire protein, as opposed to completely sequencing a protein. The identification of proteins allows for the study of protein expression (important to identify proteins that are differentially expressed under different conditions and biomarkers for disease states) as well as mapping protein interactions (which helps develop a picture of the cell architecture). Understanding the role of proteins is critical to our understanding of living systems, as proteins are the main component of biomatter and perform virtually all critical biological functions, from regulating reactions, to transport of oxygen, to providing cellular and extracellular structure. As with genomics, the burgeoning field of proteomics has resulted in the generation of information about the proteome of humans and other organisms, and, while this information is still incomplete, much of this information is and will be stored in databases. It is expected that much of our future understanding of living systems will be extracted from these genomic and proteomic databases.

In the field of clinical diagnostics, researchers focus on the identification and measurement of a wide range of analytes. The analytes of interest may be the actual drug candidates, such as in the example of bioavailability studies conducted in the course of clinical trials that reveal the extent to which a drug candidate is present throughout the organism. Alternatively, the analyte of interest may reflect a physiological response to a drug candidate, such as in the case of measuring the presence or absence of phosphorylated reaction products of kinase enzyme reactions. Because kinase enzymes are important in the growth and reproduction of cells, a high level of kinase activity is observed in patients suffering from diseases in which growth is abnormal (e.g., cancers). Drugs that result in a reduction of kinase activity are thus possible anti-cancer therapeutics, and analytical methods of detecting the efficacy of such drug candidates often focus on measuring the presence or absence of analytes in the form of kinase enzyme reaction products. These and other kinds of direct and indirect measurements of analytes of importance in clinical diagnostics and drug development depend on the existence of analytical techniques and sample presentation devices that facilitate their measurement.

The importance of sample presentation devices is by no means limited to the biomedical context. For example, researchers interested in determining the extent of environmental contamination (or remediation) need to be able to screen environmental samples of all kinds, including water, air, and soil samples. Many of the analytical techniques used to analyze such sample involve analysis of liquid samples, as is the case of water quality studies or in the case of soil samples that have been extracted by diluting in organic and/or inorganic solvents so as to remove various components. Sample presentation devices that can present liquid samples for analysis are therefore an important tool in accomplishing these kinds of analytical measurements.

In the post-September 11 world, governments are confronted with the need for platforms and analytical techniques to facilitate the detection of chemical and biological agents in both military and civil scenarios. Challenges for biowarfare detection include sample collection and distinguishing between innocuous versus toxic organisms. The current battlefield technique for bioagents utilizes pyrolysis to convert biological compounds to small molecules that can be more easily detected by mass spectometry (MS). Development of techniques that rely upon protein or peptide biomarkers is anticipated, however, because it would be more specific than currently known methods, and could be used to determine potential exposure to warfare agents in combination with breath tests, urinalysis, or blood drawing techniques. Stand-alone biosensors as alerting devices are also of great interest for use on the battlefield as well as in public places. All of these methods present challenges in sample collection, pre-treatment, and presentation of samples to detectors.

A wide variety of analytical techniques have been developed to identify and measure compounds of interest in liquid samples, such as DNA, RNA, proteins, and peptides in blood sera, environmental toxins and agents in environmental samples. While each of these analytical techniques is useful in its own way, each is at least partially dependent upon the type of sample presentation device that is employed. Thus, limitations inherent to such devices may adversely affect the measurement of compounds of interest using these analytical techniques.

Moreover, many analytical techniques focused on identifying, isolating or measuring analytes in liquid samples require that the sample undergo separate pre-processing steps—i.e., processing of the sample before it is exposed to a particular analytical technique to determine the presence and amounts of analytes of interest. For example, many protein cell extraction techniques yield complex protein mixtures and incorporate detergents and salts that interfere with mass spectral analyses that must be removed prior to analysis of the proteins. Current methods of fractionation and purification are time-consuming. Other purification methods, such as liquid chromatography and gel electrophoresis used to purify proteins, routinely involve sample recovery of volumes greater than 10 μL, necessitating additional concentration prior to analysis by various protein detection techniques (e.g., MALDI-MS). The demands of currently known analytical techniques—and the sample presentation devices used in connection with them—underscore the importance of sample purification, sample preparation, automated data acquisition, and automated data analyses.

For example, the most common and preferred type of mass spectrometry used in the field of proteomics is matrix assisted laser desorption ionization mass spectrometry (MALDI-MS). MALDI-MS is a variation of standard laser desorption time-of-flight mass spectrometry wherein proteins of relatively high molecular mass are deposited on a surface in the presence of a very large molar excess of an acidic, UV absorbing chemical matrix (for example, nicotinic acid). This technique allows for desorption of these high molecular weight labile macromolecules in the intact state. Mass spectrometry has become an important analytical tool in proteomic efforts because it provides mass accuracy, sensitive detection, and rapid analysis of minute quantities of samples at moderate cost.

However, MALDI-MS suffers from various drawbacks, particularly problems associated with sample preparation. Collectively, present day MALDI-MS sample supports suffer from a severe sample volume limitation in that they are incompatible with sample volumes in excess of 2 μL. Volumes of up to 2 μL are routinely utilized and afford dried-droplets having a diameter of from 1 mm to 2 mm. (Karas, M. and Hillenkamp, F. Anal. Chem. 1988, 60, 2299-2301, incorporated herein by reference). Because the laser irradiates only a small portion of the dried-droplet (from 0.015 mm² to 0.030 mm²) during single-site data acquisition, there is no guarantee that all proteins in a sample will be detected. In addition, the sample volume (Up to 2 μL) is significantly smaller than the volume in which samples are routinely recovered after purification necessitating their further concentration prior to MALDI-MS; for example, peptide and protein samples purified by liquid chromatographic and electrophoretic methods are routinely recovered in volumes greater than 10 μL. As a result, such samples must be further concentrated prior to MALDI-MS. Many samples also contain detergents and salts that interfere with mass spectral analyses, necessitating their removal prior to MALDI-MS.

Another drawback associated with MALDI-MS is lack of sample homogeneity. Even volumes as small as 2 μL can prove problematic owing to sample heterogeneity when the dried-droplet approach to sample application is utilized. Sample volumes in the range 0.5-2.0 μL are routinely utilized and dried, which afford dried-droplets having a diameter of from 1 mm to 2 mm. (Karas, M. and Hillenkamp, F. Anal. Chem. 1988, 60, 2299-2301, incorporated herein by reference). Consequently, only a minute portion of the dried-droplet (from 0.015 mm² to 0.030 mm²) is irradiated by the laser during single-site data acquisition. Unfortunately, even small volumes of 0.5-2.0 μL are known to result in sample heterogeneity (the heterogeneous deposition of analytes), which gives rise to significant variations in peak presence, intensity, resolution and mass accuracy when focusing the laser on different regions of the dried-droplet (Strupat, K.; Karas, M.; Hillenkcamp, F. Int'l. J. Mass Spectrom. Ion Processes 1991, 111, 89-102; Cohen, S. L. and Chait, B. T. Anal. Chem. 1996, 68, 31-37; and Amado, F. M. L.; Domingues, P.; Santana-Marques, M. G.; Ferrer-Correia, A. J.; Tomer, K. B. Rapid Commun. Mass Spectrom. 1997, 11, 1347-1352, all of which are incorporated herein by reference). These phenomena render necessary the critical inspection of the mass spectral data as well as the accumulation of a large number of single-site spectra per sample. Therefore, only a few hundred samples can be analyzed per day per instrument, and automatic data acquisition is often precluded.

It has been demonstrated that the problem of sample heterogeneity can be minimized as the spot diameter falls to the order of the laser diameter. In that case, a large portion of the sample can be irradiated simultaneously, improving sensitivity and reproducibility (Little, D. P.; Cornish, T. J.; ODonnell, M. J.; Braun, A.; Cotter, R. J.; Koster, H. Proc. Natl. Acad. Sci. U.S.A. 1997, 69, 4540-4546; and Goborn, J.; Nordhoff, E.; Mirgorodskaya, E.; Ekman, R.; Roepstorff, P. J. Mass Spectrum. 1999, 34, 105-116, incorporated herein by reference). The sample supports described in U.S. Pat. No. 6,287,872 are further described (Schuerenberg, M.; Lubbert, C.; Eickhoff, H.; Kalkum, M.; Lehrach, H; Nordhoff, E. Anal. Chem. 2000, 72, 3436-3442, incorporated herein by reference), wherein it is shown that confining the deposition of analytes to a small spot diameter not only reduces problems associated with sample heterogeneity, but also results in a significant increase in sensitivity of detection. The drawback is that to obtain this desired spot size, sample volumes have to be reduced to less than 2 μL.

To overcome these sample volume and impurity problems, researchers have employed sample supports designed or mini-columns used to pre-process samples. An example of such a sample support is commercially available as the AnchorChip™ from Bruker Daltonics GmbH. The AnchorChip™ products improve MALDI-MS sensitivity by concentrating the sample in a precisely-defined location, and specifically involve a thin layer of nonwettable hydrophobic material that carries an array of wettable hydrophilic spots. A principal limitation associated with the use of the AnchorChip™ is the requirement that the volume of liquid sample applied to each anchor be limited to from 0.50 μL to 3.0 μL (No. 1 of Eleven General Rules for Sample Preparation on AnchorChip™ Targets, see AnchorChip™ Technology, Reivsion 1.6, Bruker Daltonics GmbH, November 2000, incorporated herein by reference); the examples provided by the manufacturer in the product's literature further limit the liquid sample drop volume to either 0.5 μL or 1.0 μL. Another limitation is that both analytes and contaminants (salts, detergents) often get concentrated in the laser-irradiating region. Therefore, samples must first be desalted and/or concentrated on a ZipTip® or similar mini-column sample preparation device prior to application onto mass spectrometer sample supports, as described above. (ZipTips®, made by Millipore Corp., are micro-columns for sample concentration and desalting prepared by packing small pipette tips with reverse phase chromatographic media. (Rusconi, F.; Schmitter, J.-M.; Rossier, J.; le Maire, M. Anal. Chem. 1998, 70, 3046-3052, incorporated herein by reference)). However, the use of home-made micro-columns or commercially available ZipTips® is time consuming, adds considerable cost, has proven difficult to automate and often affords only moderate recoveries of sample material. Therefore, AnchorChips™ suffer many of the same limitations associated with other present day MALDI-MS sample supports.

An alternative technique to MALDI-MS has been developed for protein profiling of serum samples. This technique is called surface enhanced laser desorption ionization mass spectrometry (SELDI-MS), and it has produced results with respect to the discovery of biomarkers for ovarian cancer and for differentiation of prostate cancer and benign prostate hyperplasia. During SELDI-MS, analytes are first selectively retained on a sample support having a functionalized surface that acts as an affinity capture device. The retained analytes are then ionized by laser desorption at the point of capture to enable their detection without the need to effect their recovery from the retentive surface as is required for other hyphenated liquid chromatography-mass spectrometry approaches. SELDI-MS is described in U.S. Pat. Nos. 5,719,060; 5,894,063; 6,020,208; 6,027,942; 6,124,137; 6,225,047; and 6,579,719, all incorporated herein by reference. Despite the results recently reported, the SELDI-MS approach is often problematic in practice as surfaces which are optimum with respect to retention of biological analytes can exhibit less than optimum performance with respect to analyte presentation during laser desportion ionization.

Still other techniques used to isolate and purify analytes, such as proteins, have been used. For example, fractionation and purification approaches for biological samples via the time consuming techniques of 2D gel electrophoresis and multi dimensional liquid chromatography are well known, as are quicker, low sensitivity techniques such as consumable columns or pipette tips with chromatography beds. Gel electrophoresis, which serves to separate protein mixtures, can be either one or two dimensional. In 1D gel electrophoresis, also known as SDS-PAGE (sodium dodecyl sulfate-polyacrylamide gel electrophoresis), protein mixtures are separated by their molecular weight only. In 2D gel electrophoresis, also known as 2D-PAGE, mixtures are separated by their isoelectric point followed by their molecular weight. One disadvantage of the technique is that the method has poor resolution, i.e., each resolved spot might contain more than one protein. Another disadvantage is that the dyes used to see the separation do not stain all of the proteins. Liquid chromatography (LC) is known as “high performance liquid chromatography” (HPLC) or “multi-dimensional liquid chromatography,” if more than one chromatographic column is used. The advantage of LC in general is the availability of diverse column chemistries. In contrast to gel electrophoresis, which cannot efficiently separate the smaller peptides, LC can be used to separate peptide mixtures from enzymatic digests. Solid phase extraction (SPE) provides a fast way of purification and it is used in many areas, from organic synthesis to environmental sample collection. It is faster than liquid-liquid extraction or HPLC, it consumes less solvent and can be used to extract analytes from gas or liquid samples. The technique of SPE is offered in a variety of devices, such as pipette tips, columns, membranes, and 384-well plates, to mention a few.

In drug discovery, still other sample presentation devices have been developed for use in known analytical methods. For example, ADMET (Absorption Distribution Metabolism Excretion Toxicology) studies using the Empore card (http://www.3m.com/empore), a C18 RP (reverse phase) sorbent embedded in a membrane, are touted as capable of reducing the number of steps in sample purification and the potential for archiving and concentrating because the loaded samples are kept dry. Sample purification requires three steps: loading of samples on to the card, transferring the card to the eluter, and eluting 100% of the sample directly into a mass spectrometer. The Empore card could be used to load peptide digest samples on a MS if the elution volumes are kept as low as possible, otherwise low concentration peptides are below the limit of detection.

Therefore, a need exists for sample presentation devices that can be used in connection with various analytical methods to detect with high sensitivity biological and chemical moieties. Moreover, there is a need for sample presentation devices that are compatible with the sample volumes routinely recovered from liquid chromatographic and electrophoretic separations and other kinds of separation/purification techniques, that direct a liquid sample containing analytes to a confined area so as to minimize the problems associated with sample heterogeneity, that result in an increase in sensitivity of detection. The availability of such sample presentation devices would enable automated sample processing, such as, for example, on the life science industry's standard multi-well plate processors and liquid handling robots. More importantly, they also enable the direct collection and subsequent MALDI-MS analysis of chromatographic eluates. Furthermore, these capabilities would collectively enhance the throughput of the detection and measurement of biological and chemical moieties using the various analytical techniques known to those of skill in the art. These and other benefits of the present invention are described in more detail below.

### SUMMARY OF THE INVENTION

The sample presentation devices of the present invention provide attractive alternatives to known sample presentation devices used in various analytical methods used for the identification of chemical and biological entities. In addition, the present invention provides methods of making the sample presentation devices as well as methods of using them to perform a wide range of analytical measurements of analytes contained in liquid samples. The unique properties of the sample presentation devices of the present invention address many of the shortcomings (described above) associated with known analytical techniques and the sample presentation devices or containers used in connection with them.

In fields such as genomics, proteomics, drug discovery, clinical diagnostics, biosensors, and detection of environmental toxins and agents, mass spectrometry is a technique used to identify chemical and biological moieties, wherein often only very small quantities of the samples are available, and wherein rapid throughput of large numbers of samples is desirable. Other analyte detection methods, such as fluorescence polarization, immunofluorescence spectroscopy, gel chromatography, ion exchange chromatography, affinity chromatography, can also be used for high throughput detection of biological and chemical moieties, and can thus also be used in combination with the sample presentation devices of the present invention.

The sample presentation devices of the present invention provide attractive alternatives to known sample presentation devices used in various analytical methods. For example, the present invention allows for analytes to be selectively retained and concentrated on the surface of the biochip in volumes up to 100 μL. In addition, because analytes are detected from a portion of the sample presentation device that is designed to be substantially non-binding or binding resistant, they may be detected at high sensitivity as compared to direct detection on the surface of a biochip-based affinity capture device, or other sample presentation devices in which the surfaces of the devices have significant affinity for the analytes.

The present invention further minimizes the potential losses associated with the transfer of analytes from one surface to another because the present sample presentation devices, in a preferred embodiment, require only a single liquid manipulation. This, coupled with the analyte-resistant properties of the sample presentation device surfaces, results in a reduction in the loss of the analytes of interest as is the case in known methods. In contrast to SELDI-MS, the present invention does not involve desorption of bound analytes from the point of capture by an affinity capture device, but rather uses sample presentation devices wherein the desorption of analytes from a surface having no appreciable affinity or binding of the analytes to that surface.

In addition, the liquid samples can be manipulated and moved on the surfaces of the sample presentation devices of the present invention in a controlled fashion. This allows for the samples to be concentrated to an analysis zone where there is no substantial binding of analyte to the surface of the sample presentation device. Moreover, this allows the analyte-containing samples to be moved to different zones on the surfaces, each zone having different properties with respect to an analyte, which allows for purification, isolation and/or modification of the analytes prior to detection. In addition, the present invention involves sample presentation devices in which the properties of various portions of the surfaces may change in response to various chemical or physical stimuli (e.g., heat, UV radiation), such that the properties of such surfaces with respect to analytes can be manipulated during sample handling. Such changes in surface properties may be designed to be reversible or non-reversible.

These and other features of the sample presentation devices of the present invention are described in more detail below. The present invention comprises sample presentation devices, methods of making sample presentation devices, and methods of using sample presentation devices.

### Sample Presentation Devices

The present invention relates to sample presentation devices that are useful in performing analytical measurements. In one embodiment, the present invention involves sample presentation devices having surfaces with one or more zones of differing wettability with respect to various samples to be analyzed. These zones of differing wettability result in zones of differing abilities to retain, concentrate, and move analytes in liquid samples. These zones may be of various shapes and sizes, and may be continuous or discontinuous with respect to each other.

The sample presentation devices of the present invention may be comprised of distinct zones, one of which is optimal with respect to the retention of a liquid sample. The sample presentation devices of the present invention may further comprise distinct zones of wettability, one of which is optimal with respect to high sensitivity detection of analytes.

The sample presentation devices of the present invention may comprise two-dimensional or three-dimensional surfaces, each of which having two or more zones of differing wettability.

The sample presentation devices of the present invention comprise a substrate, which can be made from a variety of materials, including but not limited to, for example, glasses, semiconductors, metals, polymers (e.g., plastics), and other hydroxylated materials, e.g., SiO₂ on silicon, Al₂O₃ on aluminum, etc. Preferably, the substrate is a metal, such as gold, or semiconductor, such as silicon.

The sample presentation devices of the present invention further comprise a substrate that has been surface-modified by methods known to those of ordinary skill in the art in order to create various zones on the surface of the substrate, which zones have differing properties with respect to wettability. Such surface modifications include but are not limited to the addition of self-assembled monolayers (SAMs), polymers (linear and branched), and Langmuir-Blodgett assemblies to the substrate. Using SAMs as an example, when added to the substrate, the SAMs create a surface of the sample presentation devices to which liquid samples may be exposed. Depending on the composition of the particular SAMs used, the surfaces of the sample presentation devices of the present invention may have different properties in terms of wettability, and in terms of affinity (or lack thereof) for analytes in liquid samples. The SAMs may be added to the sample presentation devices of the present invention in a manner that creates distinct zones whose properties reflect the SAMs used in a particular zone. Other surface modification techniques known to those of skill in the art are also included in the present invention.

With respect to the kinds of zones that the surfaces of the sample presentation devices may include, they are characterized primarily by virtue of their differing wettability with respect to the sample to be analyzed, which in turn results in zones that have differing abilities to retain or bind analytes in liquid samples. These zones are broadly termed “boundary zones,” “liquid retention zones,” and “analysis zones.” The present invention only requires the presence of two types of zones, although inclusion of more than two types of zones is also contemplated. The present invention may also include more than one zone of each kind—e.g., the sample presentation devices may comprise multiple liquid retention zones, each of which may have different properties with respect to a liquid sample and/or the analytes contained therein.

A first type of zone is termed a “boundary zone” and involves a substantially non-wettable zone with respect to the sample to be analyzed. The boundary zone is the zone with the highest contact angle with respect to the sample in comparison to the other zones.

A second type of zone, termed the “liquid retention zone,” is relatively more wettable in comparison to the boundary zone with respect to the sample to be analyzed (and is relatively less wettable than the analysis zone, described below). The liquid retention zone has a contact angle relatively lower than the contact angle of the boundary zone (and a contact angle relatively higher than the contact angle of the analysis zone, described below). The liquid retention zone can also have equal or lower contact angle than the analysis zone initially, but because of chemical or physical stimuli, the liquid retention zone may assume a higher contact angle than the analysis zone prior to the chemical or physical stimuli, which results in the liquid sample being directed to one zone preferentially over another.

The liquid retention zone can be of two subtypes. In one subtype, the liquid retention zone is designed to operate for liquid sample retention purposes, while being substantially analyte binding resistant. In a second subtype, the liquid retention zone is designed to retain a liquid sample, but also to substantially bind analytes within a liquid sample, and can thus be termed a “capture zone” in that it captures the analytes. This second subtype may also include a surface that is substantially analyte binding but that becomes substantially non-binding upon being subjected to chemical or physical stimuli, such as, for example, UV radiation, electricity, or heat.

A third type of zone is termed the “analysis zone” and is the zone that is the most wettable (and has the lowest contact angle) with respect to the sample in comparison to the other zones. The analysis zone is designed to be analyte binding resistant. The analysis zone may be optimized in terms of size, shape, and surface properties to enhance the sensitivity of the analysis of the desired analytes.

The liquid capacity of the sample presentation devices of the present invention is dependent on the sizes of the zones. For a 3 mm diameter circular zone, the liquid capacity can be up to about 100 μl. The sample presentation devices can contain this amount of liquid sample without the need for physical boundaries, reservoirs, or wells. The various zones can be precisely positioned in order to facilitate or be compatible with high throughput automation on various analytical instruments, such as, for example, mass spectrometry instruments.

In another embodiment of the sample presentation devices of the present invention, the sample presentation devices can be termed “target chips,” and abbreviated Tn, where “n” is a numerical designation referring to the number of distinct zones on the surface of the sample presentation device, where “n” can be any number from 2 to infinity. Thus, for example, a T2 target chip has two zones, a T3 target chip has three zones, etc. The present invention contemplates sample presentation devices containing many more than 2 or 3 zones and is not limited in any way to a specific number of zones. As the number of zones increases, the overall effect approaches a gradient. Target chips are sample presentation devices comprised of one or more zones that are designed to be resistant to analyte binding.

With respect to a T2 target chip, for example, the sample presentation device comprises two zones—i.e., a boundary zone and an analysis zone. The surfaces of the zone that contacts the liquid sample are designed to be analyte binding resistant—i.e., the analysis zone is analyte binding resistant. The surfaces of the zone that contacts the liquid sample effectively confine the analytes during the drying step before analysis.

With respect to a T3 target chip, the sample presentation device comprises three zones—i.e., a boundary zone, a liquid retention zone, and an analysis zone. The surfaces of the zones that contact the liquid sample are designed to be analyte binding resistant—i.e., the liquid retention zone and the analysis zone are analyte binding resistant. The surfaces of the zones that contact the liquid sample effectively concentrate the analytes to the analysis zone during the drying step.

The sample presentation devices of the present invention may thus comprise distinct zones, each of which exhibits a minimum of adsorption with respect to analytes.

In another embodiment of the sample presentation devices of the present invention, the sample presentation devices can be termed “capture chips” or “capture/concentrate chips,” and abbreviated Xn, where “n” is a numerical designation referring to the number of zones on the surface of the sample presentation device, where “n” can be any number from 2 to infinity. Thus, for example, an X2 capture chip has two zones, an X3 capture chip has three zones, etc. The present invention contemplates sample presentation devices containing many more than 2 or 3 zones and is not limited in any way to a specific number of zones. As the number of zones increases, the overall effect approaches a gradient. Capture chips and capture/concentrate chips are sample presentation devices comprised of one or more zones that are designed to bind analytes.

With respect to an X2 capture chip, for example, the sample presentation device comprises two zones—i.e., a boundary zone and a capture zone. The surfaces of the zones that contact the liquid sample are designed to capture the analytes—i.e., the capture zone binds the analytes—based on the chemical or biological properties of the surfaces of the capture zone. The surfaces of the zones that contact the liquid sample effectively confine the analytes during the drying step before analysis.

With respect to an X3 capture/concentrate chip, the sample presentation device comprises three zones—i.e., a boundary zone, a capture zone, and an analysis zone. The boundary zone is designed to be substantially non-wettable. The capture zone is designed to capture and bind analytes. The analysis zone is designed to be analyte binding resistant. Analytes are transferred between the capture and analysis zones, which is done prior to analysis by one of the various known analytical detection methods. The surface of the analysis zone that contains the liquid sample effectively confines the analytes during the drying step before analysis. The transfer of the liquid sample from the capture zone to the analysis zone may be accomplished by virtue of the properties of the surface of the capture zone—i.e., if the capture zone has a lower degree of wettability than the analysis zone, the liquid sample will move from the capture zone to the analysis zone without physical intervention. Alternatively, the capture zone may be designed such that its properties may be changed in response to chemical or physical stimuli (e.g., heat, UV radiation), causing the capture zone to have a lower degree of wettability than the analysis zone, and thus causing the liquid sample to move from the capture zone to the analysis zone.

In yet another embodiment of the sample presentation devices of the present invention, the sample presentation devices can be combinations of the above-described target and capture chips. In this embodiment, the sample presentation devices are comprised of surfaces having different functionality. These kinds of sample presentation devices may involve the transfer of a liquid sample from one zone to another by mechanical means (e.g., via pipetting)or otherwise (e.g., via the differences in wettability between zones). As an example, a “capture-transfer-concentrate chip,” abbreviated X2-transfer-T3, is a sample presentation device comprised of both an X2 chip comprised of two zones (i.e., a boundary zone and a capture zone), as well as a T3 chip comprised of three zones (i.e., boundary zone, liquid retention zone, and analysis zone). A transfer (mechanical or otherwise) of the analyte occurs between the capture zone of the X2 chip and the liquid retention zone of the T3 chip. In addition, the embodiments of the sample presentation devices that involve combinations of capture zones and liquid retention zones may further be used in a combinatorial manner to isolate, concentrate, purify, and modify analytes in liquid samples prior to their detection. So, for example, a liquid sample may be placed onto a T2 chip such that the analytes in the sample are confined in the analysis zone. That sample may then be transferred to an X3 chip that contains a boundary zone, a capture zone, and an analysis zone. In this example, the capture zone may be designed to bind (and thus remove) lipid moieties from the liquid sample, such that when the sample is applied to the X3 chip, it moves from the boundary zone to the capture zone (which has a higher degree of wettability), the lipid moieties in the sample bind to the surface of the capture zone, and the remaining sample moves to the analysis zone (because it has the highest degree of wettability). In this example, the liquid sample is confined on the T2 chip, and then the lipids are moved on the X3 chip, such that the final sample that is analyzed from the analysis zone is concentrated and purified of lipids. Because the capture zones can be designed to bind a multitude of different analytes, and because various combinations of any of these zones may be used, sample presentation devices having a vast range of purification, concentration, isolation, and modification capabilities (vis-à-vis one or more analytes) can be created.

The mechanism of transfer of liquid samples from one sample presentation device to another may vary. Using the above example, the concentrated sample from T2 may be removed mechanically (e.g., by pipetting) and placed on a separate X3 sample presentation device. Alternatively, the T2 and X3 sample presentation devices may be connected by a zone, the wettability of which may be changed in response to chemical or physical stimuli (e.g., UV radiation), such that the concentrated sample in the analysis zone of the T2 sample presentation device is transferred to the capture zone of the X3 device when the exposure of a zone between them to UV radiation results in a wettability that is higher than the analysis zone of the T2 device but lower than that of the capture zone of the X3 device, such that the sample moves from T2 to X3. Again, with a vast number of surfaces (having different wettability and analyte binding properties) and configurations thereof, sample presentation devices having a vast range of purification, concentration, isolation, and modification capabilities (vis-a-vis one or more analytes) can be created.

The sample presentation devices of the present invention further provide zones of different wettability having different shapes or patterns. For example, in one embodiment, a sample presentation device may have zones in the form of concentric circles, with the center zone being the analysis zone, surrounded by the liquid retention zone, surrounded by the boundary zone. Because the zones can be created using a various photo-patterning techniques, and because known photo-patterning techniques provide for tremendous variation in the resulting patterns, there is a vast range of possible shapes, patterns, and configurations of the various zones. Moreover, the various properties of the different zones of wettability allow for the creation of sample presentation devices capable of directing analytes to single or multiple specified or pre-determined locations on the surfaces (e.g., addressable sites, lanes, or fields).

The sample presentation devices of the present invention are suitable for the handling of both biological and non-biological liquid samples. They are also suitable for application in a wide range of analyte detection methods, for example, including but not limited to, mass spectrometry, various chromatographic methods, immunofluorescence spectroscopy, and other known analytical methods of detecting and measuring analytes in liquid samples.

Each of the above-described variations is designed to allow for maximum flexibility in design and use of sample presentation devices having enhanced capability to present analytes for detection and analysis over known methods. Thus, the sample presentation devices of the present invention have the capability of directing analytes to an analysis zone designed to enhance high sensitivity detection of analytes. The sample presentation devices of the present invention thus afford improved deposition of analytes.

### Fabrication of Sample Presentation Devices

Still other embodiments of the invention include methods for creating or fabricating the sample presentation devices described above.

In an embodiment in which the surfaces are comprised of self-assembled monolayers (SAMs) which form distinct zones depending on differences between the SAMs used, the sample presentation devices of the present invention may comprise various SAM zones that are created by known photo-patterning techniques. Accordingly, the present invention further includes methods of creating sample presentation devices comprised of SAMs using, as one preferred method, photo-patterning techniques.

The surface of the substrate of the sample presentation device of the present invention is typically modified or patterned by methods known to those of skill in the art. As an example, the substrate's surface can be modified or patterned by means of applying self-assembled monolayers (SAMs), which modify the surface of the substrate of the sample presentation device and whose exposed surfaces may impart particular chemistries to the substrate. Selection of various SAMs, including 1°, 2°, 3°, or 4° compositions, for a particular substrate provides the surface of the substrate with unique surface characteristics and properties. In particular, application of multiple SAMs results in the patterning of the substrate so that it contains a plurality of zones, each zone having different surface characteristics and properties. Methods of patterning the SAMs are known in the art, and include UV photo-patterning, photolithographic patterning, microstamping, electron-beam patterning, and reactive-ion etching.

The zones that are created on the surface of the substrate can be in any shape, with circular shapes being preferred. In addition, the zones can be either continuous or discontinuous with respect to other zones—i.e., the zones can all be contiguous with each other or one or more zones can be discontiguous with one or more other zones. The zones that are created on the surface of the substrate of the sample presentation devices preferably have a plurality of zones of differing wettability with respect to the sample to be analyzed.

As another embodiment of the invention, methods of fabricating sample presentation devices that are capable of precisely positioning analytes so as to facilitate automated data acquisition are provided.

### Uses and Applications of Sample Presentation Devices

In another embodiment, the sample presentation devices of the present invention find many uses in combination with various analytical techniques and procedures. Thus, the present invention includes methods for using the aforementioned sample presentation devices. More specifically, present invention includes methods of using the sample presentation devices of the present invention to identify the presence of analytes in a sample, and to analyze a plurality of samples, either on a sample presentation device or on a plurality of sample presentation devices.

Virtually any analytical method that permits the detection, identification, or measurement of analytes in a liquid sample can be used in combination with the sample presentation devices of the present invention. Examples of such analytical methods include but are not limited to, MALDI-MS or electrospray ionization MS. The sample presentation devices are particularly well suited to us in combination with high throughput analytical measurement techniques, such as, for example, for use in MALDI-MS in which the sample presentation device analysis zones are configured in such fashion as to promote high throughput data acquisition.

The sample presentation devices of the present invention may also be used to manipulate liquid samples, and the analytes contained therein. Based on the differing wettability properties and capture properties that the surfaces of the sample presentation devices may be designed to have, the sample presentation devices may be designed to manipulate, concentrate, position, store, transfer (with and without mechanical intervention), recover (with or without mechanical intervention), analyze, modify or process (via use of analyte modifying reagents on the sample presentation devices), or fractionate liquid samples or the analytes contained therein. Moreover, because the sample presentation devices of the present invention may be designed to accomplish any of these functions in response to chemical or physical stimuli (e.g., heat, UV radiation, pressure, electromagnetic radiation), the sample presentation devices of the present invention may accomplish these functions reversibly or irreversibly, and may further perform various combinations of these functions in response to external forces.

Any liquid sample (and analytes) can be used in connection with the sample presentation devices of the present invention. For example, the present invention can be used to analyze fractions recovered from liquid chromatography. The present invention can be used to analyze enzymatic digests prepared from either protein spots excised from 2D gel electrophoresis or from fractions collected from affinity chromatography (i.e., ICAT (Isotope-Coded Affinity Tags)). The present invention can also be used to analyze samples recovered from biosensors. The present invention can also be used for 1:1 sample transfer with standard multi-well format robotics and assays. Indeed, the sample presentation devices of the present invention can be used to handle and manipulate liquid samples obtained from virtually any source, whether such samples are the result of laboratory experiment (such as the enzymatic digest and biosensor sample examples identified above), obtained from the environment (such as a water quality sample from a river), or obtained directly from living organisms (such as a human urine sample).

The present invention can also be used for storage of samples for archival purposes or for further analysis. In other words, the detection and analysis of the analytes contained in liquid samples need not occur immediately following transfer of the liquid sample to the analysis zone.

Thus, various embodiments of the present invention provide for sample presentation devices that serve a variety of liquid-handling functions, including but not limited to sample/analyte handling, as well as liquid deposition, retention, transfer, locating and re-locating, and storage.

### Features and Advantages

In addition to the many features and advantages of the present invention described in the summary of the invention section above, additional features and advantages include at least the following:

Analytical methods to detect analytes present in a liquid sample, such as MALDI-MS, can be performed from a single surface that is substantially analyte non-binding, resulting in increased sensitivity of analysis, increased reproducibility of results, and comparable results from different capture zones.

With respect to sample liquid handling, increased sample volumes—up to about 100 μl for a 3 mm diameter zone—can be analyzed, surfaces can be patterned having SBS (Society for Biomolecular Screening) standard well formats (i.e., 96/384/1536 well formats), and thus are able to be interfaced with common robotics and other high throughput analytical methods.

Increased throughput for the various analytical methods (e.g., MALDI-MS) can be achieved, in that zones are precision placed for high throughput data acquisition. With respect to MALDI-MS, the analysis zone is of optimal size (i.e., less than 2 mm², and preferably less than 1 mm²). The sample/matrix has improved crystallization, leading to improved ionization consistency within the analysis zone. The smaller analysis zone as compared to dried spot analysis results in less area to interrogate, resulting in high throughput of analysis.

The sample presentation devices of the present invention enable analysis of diluted samples by means of the concentration of analyte in the analysis zone.

Separation of analytes in a liquid sample is possible without the need for multiple separation steps, such as with binding analytes to an ion exchange chromatography column and then having to isolate the analytes from the column in a subsequent wash step. Indeed, by using SAMs with different surface chemistries designed to bind to different analytes, highly specific isolation and purification of particular analytes is possible.

A wide array of liquid samples and analytes can be handled by the sample presentation devices of the present invention, which avoid the shortcomings of known presentation devices and analytical methods described above. While the sample presentation devices of the present invention are particularly well suited to use in the proteomics field and laser desorption ionization mass spectroscopy, as is described in detail below the utility of the claimed devices is not in any way limited to only that field.

### BRIEF DESCRIPTION OF THE FIGURES

FIG. 1a depicts a sample presentation device of the present invention, wherein the central analysis zone and the surrounding liquid retention zone are concentric with respect to one another, and wherein the liquid retention zone is surrounded by a boundary zone.

FIG. 1b depicts a cross-sectional view of the sample presentation device depicted in FIG. 1a.

FIG. 2 depicts the surface of a sample presentation device of the present invention, wherein the surface is further comprised of 16 pairs of analysis zones and liquid retention zones, wherein the analysis zones and liquid retention zones are concentric with respect to one another, and wherein pairs of analysis zones and liquid retention zones are surrounded by a common boundary zone. In this instance, the sample presentation device is organized on geometries corresponding to standard 96-well plate.

FIG. 3 depicts the surface of a sample presentation device of the present invention, wherein a portion of the analysis zone and liquid retention zone are contiguous with respect to one another, wherein those portions of the analysis and liquid retention zones that are not contiguous with respect to one another are surrounded by a common boundary zone, and wherein the surface area of the analysis zone is smaller than the surface of the liquid retention zone.

FIG. 4a depicts the surface of a sample presentation device of the present invention, wherein the shape of the analysis zone has been designed to facilitate the automated acquisition of mass spectral data. FIG. 4b depicts an enlargement of the analysis zone indicating 36 regions which measure approximately 100 μm², and which correspond to the individual regions that may be sampled by the laser during mass spectrometry.

FIG. 5 depicts the surface of a sample presentation device of the present invention, wherein the surface is further comprised of 96 pairs of analysis zones and liquid retention zones, wherein the analysis zones and liquid retention zones are concentric with respect to one another, and wherein pairs of analysis zones and liquid retention zones are surrounded by a common boundary zone. In this instance, the sample presentation device is organized on geometries corresponding to a standard 96-well plate. The liquid retention zone is elongated to maximize liquid-holding capacity and minimize the distance between adjacent zones. A serpentine pattern is overlaid on the first two rows of the sample presentation device to indicate the path described by deposition of a liquid stream of chromatographic eluate during automated fraction collection.

FIGS. 6a through 6h illustrate the steps involved in fabrication of a sample presentation device of the present invention, when alkylthiols on gold are utilized for surface modification and UV-photopatterning is exploited for surface patterning.

FIGS. 7a through 7l illustrate the steps involved in fabrication of a sample presentation device of the present invention, when alkylthiols on gold are utilized for surface modification and photolithography is exploited for surface patterning.

FIGS. 8a through 8l illustrate the steps involved in fabrication of a sample presentation device of the present invention, when alkylsilanes on silicon are utilized for surface modification and photolithography is exploited for surface patterning.

FIGS. 9a through 9f depict various stages during the process whereby a large volume of aqueous sample deposited on the surface of a sample presentation device of the present invention dries within the area corresponding to the analysis zone.

FIGS. 10a through 10d depict the surface and drop drying characteristics associated with a sample presentation device having a liquid retention zone and no analysis zone. FIGS. 10e through 10h depict the surface and drop drying characteristics associated with a sample presentation device having an analysis zone and no liquid retention zone.

FIGS. 11a through 11h depict images recorded on a video contact angle apparatus during the drying of a drop on the surface of a sample presentation device of the present invention, wherein the analysis zone measures 0.6 mm diameter and the liquid retention zone measures 1.5 mm diameter.

FIG. 12 is a graph that summarizes the contact angle, drop width and drop height associated with the images depicted in FIGS. 11a through 11h.

FIG. 13 illustrates a sample presentation device of the present invention with liquid volumes of from 5 μL to 70 μL deposited thereupon.

FIG. 14a illustrates, a sample presentation device of the present invention taken immediately after liquid drops of from 5 μL to 40 μL were deposited thereupon. Each of the liquid drops contained an equivalent amount of alpha-cyano-4-hydroxycinnamic acid (HCCA).

FIG. 14b illustrates the HCCA having been concentrated and directed to the analysis zone due to sample drying on the sample presentation device depicted in FIG. 14a. A visual reference to the concentric zones is superimposed above the dried HCCA.

FIG. 15 illustrates a variation of a process for extracting desired analytes for analysis utilizing a sample presentation device.

FIG. 16a illustrates desired analytes being bound on the capture zone of the sample presentation device. Illustrations of associated mass spectrometry spectrums are also shown.

FIG. 16b illustrates desired analytes being focused onto the analysis region of the sample presentation device. Illustration of an associated mass spectrometry spectrum is also shown.

FIG. 17a illustrates a mass-spectrometry spectrum from a sample contaminated with 1M NaCl which has been processed with a capture chip.

FIG. 17b illustrates a mass-spectrometry spectrum from a sample contaminated with 1M Urea which has been processed with a capture chip.

FIG. 17c illustrates a mass-spectrometry spectrum from a sample contaminated with 1M TRIS which has been processed with a capture chip.

FIG. 17d illustrates a mass-spectrometry spectrum from a sample contaminated with 1M NaCl which has been processed with a capture chip.

FIG. 18a illustrates a mass-spectrometry spectrum of a sample that was directly applied on an X3 chip.

FIG. 18b illustrates a mass-spectrometry spectrum of the sample that was applied on a T3 chip after ZipTip filtering.

FIG. 18c illustrates a mass-spectrometry spectrum of the sample that was directly applied on a stainless steel surface.

FIG. 19a illustrates a spectrum derived from an X3-type surface using TFA protocol.

FIG. 19b illustrates a spectrum derived from an X3-type surface using MOPS protocol.

FIG. 19c illustrates a spectrum of a clean digest derived from a T3-type surface.

FIG. 20 is an FTIR spectrum which confirms the presence of the NHS-ester group.

FIG. 21 shows the antigen detection results for the sample presentation device utilizing different antibodies (Anti ACTH C-terminus vs. Anti ACTH N-terminus vs. Non-specific Mouse IgG). Two types of antigens (ACTH 18-39 (C-terminal) and ACTH 1-39 (full-length) were utilized in two separate tests for each antibody surfaces. The + and − signs indicate whether the analyte was detected. The concentration (in parentheses) is an actual antigen solution concentration tested that resulted in the corresponding positive/negative result.

FIG. 22 illustrates the spectrum for 125 fmol of β-casein in-gel digest on the IMAC-Fe X3 chip created in example XXVI.

FIG. 23 illustrates the spectrum obtained from a 100 fmol phosphorylase b solution digest +5 fmol β-casein solution digest placed on a T3 chip.

FIG. 24 illustrates the spectrum obtained from a 100 fmol phosphorylase b solution digest +5 fmol β-casein solution digest after processing on the IMAC—Fe X3 chip created in example XXVI.

FIG. 25 illustrates detection of phosphotyrosine-containing peptide in sample solutions with varying concentrations of the peptide (50 fmol, 5 fmol, and 500 amol) the IMAC—Fe X3 chip created in example XXVI. (* indicates the position of the phosphotyrosine peptide's spectrum peak).

FIG. 26 illustrates the spectrum from a mixture having both His-tagged (m/Z˜9500) and non-His-tagged ubiquitins (m/Z˜8500) after the mixture had been process with the IMAC—Ni X3 chip created in example XXVII. As seen in FIG. 26, only the His-tagged variant of the ubiquitins is visible.

FIG. 27a illustrates one variation of a sample presentation device comprising three concentric circles. The surfaces of the circles are configured to promote movement of liquids toward the center region.

FIG. 27b illustrates another variation of a sample presentation device, in which a “drop zone” is provided for receiving a sample liquid.

FIG. 27c illustrates another variation of a sample presentation device with four integrated drop zones.

FIG. 27d illustrates another variation of the sample presentation device comprising a liquid transport region for transporting liquid across the surface of the sample presentation device.

FIG. 27e illustrates yet another variation of the sample presentation device comprising a drop zone, a liquid transport region, a liquid retention zone, and an analysis zone. One or more of the regions may have a chemically active surface for binding or reacting with an analyte in the sample solution to be processed by the sample presentation device.

FIG. 28a illustrates a subsection of an array of processing sites on one variation of a sample presentation device.

FIG. 28b illustrates one of the individual processing sites from the array shown in FIG. 28a.

FIG. 29 illustrates one application where a photo-emitter and a photo-detector are utilized to measure the analyte presented on the sample presentation device.

FIG. 30 illustrates another variation of a system for detecting/measuring analyte on a sample presentation device. In this variation, the presentation device is utilized with an apparatus which ionizes the analytes on the sample presentation device and directs the ionized particles toward the detector.

FIG. 31 illustrates another application where energy is transmitted through the sample presentation device to analyze the sample presented on the sample presentation device.

### DETAILED DESCRIPTION OF THE PREFERRED EMBODIMENTS

The following detailed description should be read with reference to the drawings, in which identical reference numerals refer to like elements through out the different figures. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

### Definitions

Unless defined otherwise, all technical and scientific terms used herein have the meaning commonly understood by a person skilled in the art to which this invention belongs. As used herein, the following terms have the meanings ascribed to them unless specified otherwise.

“Analyte(s)” refers to component(s) of a sample which is desirably detected. The term can refer to a single component or to multiple components in the sample.

“Sample(s)” refers to any material derived from a biological or non-biological sources which is presented on the surface of a sample presentation device. The samples may be applied to the sample presentation devices in their original, untreated form and/or after treatments, including but not limited to modification, fractionation, extraction, and concentration. The samples of the present invention can be liquid or non-liquid samples.

“Substrate” refers to a material that is capable of presenting or supporting a surface.

“Surface” refers to the exterior or upper boundary of a body or a substrate.

“Substantially non-binding” or “binding resistant” or “analyte binding resistant” refers to the property of certain surfaces used in connection with the sample presentation devices of the present invention that do not exhibit appreciable affinity or binding of an analyte to a surface. While some binding may occur, these surfaces are specifically designed to minimize binding to levels below the limit of detection of the analysis method employed.

“Surface tension” refers to a property of liquids in which a liquid drop deposited on a surface tends to contract to the smallest possible contact area because of unequal molecular cohesive forces near the surface.

“Wettability” refers to the degree to which a solid surface is wetted by a liquid sample. Unless otherwise specified, liquid samples are aqueous in nature.

“Contact angle” refers to the angle between the plane of the solid surface and the tangential line to the liquid drop boundary originating at the point of three phase contact (solid/liquid/vapor).

“Matrix” refers to materials used in mass spectroscopy techniques, such as MALDI-MS or SELDI-MS, for absorbing the energy of the laser and transferring that energy to analyte molecules, enabling ionization of labile macromolecules. In SELDI-MS, the matrix is referred to as “EAM” or “energy absorbing molecule.” Reagents frequently used as matrices for the detection of biological analytes include but are not limited to trans-3,5-dimethoxy-4-hydroxycinnamic acid (sinapinic acid, SA), α-cyano-4-hydroxycinnamic acid (HCCA) and 2,5-dihydroxybenzoic acid (DHBA). Other suitable matrices are known to those skilled in this art.

“SAM” refers to self-assembled monolayers. SAMs are molecular assemblies that are formed spontaneously by the immersion of an appropriate substrate into a solution of an active surfactant in an organic solvent.

It must also be noted that, as used in this specification and the appended claims, the singular forms “a,” “an” and “the” include plural referents unless the context clearly dictates otherwise. Thus, for example, the term “a molecule” is intended to mean a single molecule or a combination of molecules, “a fluid” is intended to mean one or more fluids, or a mixture thereof.

### Description of the Sample Presentation Devices

The following description of the sample presentation devices of the present invention provides a more detailed understanding than set forth above in the summary of the invention. However, the sample presentation devices of the present invention are further described by reference to the figures, the methods of fabricating the sample presentation devices of the present invention, and the uses and applications of the sample presentation devices of the present invention, each of which is described in detail below.

As noted above, the sample presentation devices of the present invention provide attractive alternatives to known sample presentation devices used in various analytical methods used for the identification of chemical and biological entities. In addition, the present invention provides methods of making the sample presentation devices as well as methods of using them to perform a wide range of analytical measurements of analytes contained in liquid samples. The unique properties of the sample presentation devices of the present invention address many of the shortcomings (described in the background section above) associated with known analytical techniques and the sample presentation devices or containers used in connection with them.

More specifically, the sample presentation devices of the present invention provide attractive alternatives to known sample presentation devices used in a wide range of analytical methods. They have additional benefits, such as, for example, allowing for analytes in a liquid sample to be selectively retained and concentrated on the surface of the biochip in volumes up to 100 μL. In addition, because analytes are detected from a portion of the sample presentation device that is designed to be substantially non-binding or binding resistant, they are detected at high sensitivity as compared to direct detection on the surface of a biochip-based affinity capture device, or other sample presentation devices in which the surfaces of the devices have significant affinity for the analytes.

The present invention further minimizes the potential losses associated with the transfer of analytes from one surface to another because the present sample presentation devices, in a preferred embodiment, require only a single liquid manipulation. This, coupled with the analyte-resistant properties of the sample presentation device surfaces, results in a reduction in the loss of the analytes of interest.

In addition, because the analytes are not bound to affinity capture devices as in, for example, SELDI-MS biochips, the liquid samples can be manipulated and moved on the surfaces of the sample presentation devices of the present invention in a controlled fashion. This allows for the samples to be concentrated to an analysis zone where there is no substantial binding of analyte to the surface of the sample presentation device. Moreover, this allows the analyte-containing samples to be moved to different zones on the surfaces, each zone having different properties with respect to an analyte, which allows for purification, isolation and/or modification of the analytes prior to detection.

The present invention involves sample presentation devices in which the properties of various portions of the surfaces may change in response to various chemical or physical stimuli (e.g., heat, UV radiation), such that the properties of such surfaces with respect to analytes can be manipulated during sample handling. Such changes in surface properties may be designed to be reversible or non-reversible.

The present invention thus relates to sample presentation devices that are useful in performing analytical measurements. In one embodiment, the present invention involves sample presentation devices having surfaces with one or more zones of differing wettability with respect to various samples to be analyzed. These zones of differing wettability result in zones of differing abilities to retain, concentrate, and move analytes in liquid samples. These zones may be of various shapes and sizes, and may be continuous or discontinuous with respect to each other. The sample presentation devices of the present invention may comprise two-dimensional or three-dimensional surfaces, each of which having two or more zones of differing wettability.

The sample presentation devices of the present invention comprise a substrate, which can be made from a variety of materials, including but not limited to, for example, glasses, silicates, semiconductors, metals, polymers (e.g., plastics), and other hydroxylated materials, e.g., SiO₂ on silicon, Al₂O₃ on aluminum, etc. Preferably, the substrate is a metal, such as gold, or a semiconductor, such as silicon. The sample presentation devices of the present invention further comprise a substrate that has been surface-modified by methods known to those of ordinary skill in the art in order to create various zones on the surface of the substrate, which zones have differing properties with respect to wettability. Such surface modifications include but are not limited to the addition of self-assembled monolayers (SAMs), polymers (linear and branched), and Langmuir-Blodgett assemblies to the substrate. Using SAMs as an example, when added to the substrate, the SAMs create a surface of the sample presentation devices to which liquid samples may be exposed. Depending on the composition of the particular SAMs used, the surfaces of the sample presentation devices of the present invention may have different properties in terms of wettability, and in terms of affinity (or lack thereof) for analytes in liquid samples. The SAMs may be added to the sample presentation devices of the present invention in a manner that creates distinct zones whose properties reflect the SAMs used in a particular zone. Other surface modification techniques known to those of skill in the art are also included in the present invention.

The sample presentation devices of the present invention are comprised of distinct zones, one of which is optimal with respect to the retention of a liquid sample. The sample presentation devices of the present invention may further comprise distinct zones of wettability, one of which is optimal with respect to high sensitivity detection of analytes.

With respect to the kinds of zones that the surfaces of the sample presentation devices may include, they are characterized primarily by virtue of their differing wettability with respect to the sample to be analyzed, which in turn results in zones that have differing abilities to retain or bind analytes in liquid samples. These zones are broadly termed “boundary zones,” “liquid retention zones,” and “analysis zones.” The present invention only requires the presence of two types of zones, although inclusion of more than two types of zones is also contemplated. The present invention may also include more than one zone of each kind—e.g., the sample presentation devices may comprise multiple liquid retention zones, each of which may have different properties with respect to a liquid sample and/or the analytes contained therein. The various zones can be precisely positioned in order to facilitate or be compatible with high throughput automation on various analytical instruments, such as, for example, mass spectrometry instruments.

The “boundary zone” involves a substantially non-wettable zone with respect to the sample to be analyzed. The boundary zone is the zone with the highest contact angle with respect to the sample in comparison to the other zones.

The “liquid retention zone” is relatively more wettable in comparison to the boundary zone with respect to the sample to be analyzed (and is relatively less wettable than the analysis zone, described below). The liquid retention zone has a contact angle relatively lower than the contact angle of the boundary zone (and a contact angle relatively higher than the contact angle of the analysis zone, described below). The liquid retention zone can also have equal or lower contact angle than the analysis zone initially, but because of chemical or physical stimuli, the liquid retention zone may assume a higher contact angle than the analysis zone prior to the chemical or physical stimuli, which results in the liquid sample being directed to one zone preferentially over another. Moreover, the liquid retention zone can be of two subtypes. In one subtype, the liquid retention zone is designed to operate for liquid sample retention purposes, while being substantially analyte binding resistant. In a second subtype, the liquid retention zone is designed to retain a liquid sample, but also to substantially bind analytes within a liquid sample, and can thus be termed a “capture zone” in that it captures the analytes. This second subtype may also include a surface that is substantially analyte binding but that becomes substantially non-binding upon being subjected to chemical or physical stimuli, such as, for example, UV radiation, electricity, or heat.

The “analysis zone” is the zone that is the most wettable (and has the lowest contact angle) with respect to the sample in comparison to the other zones. The analysis zone is designed to be analyte binding resistant. The analysis zone may be optimized in terms of size, shape, and surface properties to enhance the sensitivity of the analysis of the desired analytes.

Among other benefits, the sample presentation devices of the present invention are able to retain and handle liquid sample volumes that are larger than other biochips used in sample handling, due to the differences in wettability between zones. While the liquid capacity of the sample presentation devices of the present invention is dependent on the sizes of the zones; for a 3 mm diameter circular zone, the liquid capacity can be up to about 100 μL, and at least up to about 70 μL. The sample presentation devices can contain this amount of liquid sample without the need for physical boundaries, reservoirs, or wells.

In another embodiment of the sample presentation devices of the present invention, the sample presentation devices can be termed “target chips,” and abbreviated Tn, where “n” is a numerical designation referring to the number of distinct zones on the surface of the sample presentation device, where “n” can be any number from 2 to infinity. Thus, for example, a T2 target chip has two zones, a T3 target chip has three zones, etc. The present invention contemplates sample presentation devices containing many more than 2 or 3 zones and is not limited in any way to a specific number of zones. As the number of zones increases , the overall effect approaches a gradient. Target chips are sample presentation devices comprised of one or more zones that are designed to be resistant to analyte binding. With respect to a T2 target chip, for example, the sample presentation device comprises two zones—i.e., a boundary zone and an analysis zone. The surfaces of the zone that contacts the liquid sample are designed to be analyte binding resistant—i.e., the analysis zone is analyte binding resistant. The surfaces of the zone that contacts the liquid sample effectively confine the analytes during the drying step before analysis. With respect to a T3 target chip, the sample presentation device comprises three zones—i.e., a boundary zone, a liquid retention zone, and an analysis zone. The surfaces of the zones that contact the liquid sample are designed to be analyte binding resistant—i.e., the liquid retention zone and the analysis zone are analyte binding resistant. The surfaces of the zones that contact the liquid sample effectively concentrate the analytes to the analysis zone during the drying step. The sample presentation devices of the present invention may thus comprise distinct zones, each of which exhibits a minimum of adsorption with respect to analytes.

In another embodiment of the sample presentation devices of the present invention, the sample presentation devices can be termed “capture chips” or “capture/concentrate chips,” and abbreviated Xn where “n” is a numerical designation referring to the number of zones on the surface of the sample presentation device, where “n” can be any number from 2 to infinity. Thus, for example, an X2 target chip has two zones, an X3 target chip has three zones, etc. The present invention contemplates sample presentation devices containing many more than 2 or 3 zones and is not limited in any way to a specific number of zones. As the number of zones increases, the overall effect approaches a gradient. Capture chips and capture/concentrate chips are sample presentation devices comprised of one or more zones that are designed to bind analytes. The moieties responsible for capturing analytes typically comprise specific surface modifications that are designed as the distinguishing feature of the capture zone. These surface modifications may comprise biological and chemical moieties that bind analytes specifically (such as monoclonal antibodies) or non-specifically (such as charged groups that bind on the basis of electrostatic attraction) or any combination of such attractive forces. In addition to the ability to capture an analyte of interest, these surface modifications may also retain the analytes in a liquid sample to permit subsequent modification. So, for example, a sample presentation device of the present invention that comprises a capture zone in which the surface modification is a monoclonal antibody may bind a complimentary antigen from a liquid sample and retain that antigen while the rest of the liquid sample moves to another part of the surface of the device, through either physical transfer or differences in wettability. The retained antigen may be modified via the addition of other compounds to the capture zone of the sample presentation device (e.g., the addition of an enzyme that cleaves off a part of the antigen). The modified antigen can then be transferred to another portion of the sample presentation device for further handling, or removed from the device for analysis by known techniques.

With respect to an X2 capture chip, for example, the sample presentation device comprises two zones—i.e., a boundary zone and a capture zone. The surfaces of the zones that contact the liquid sample are designed to capture the analytes—i.e., the capture zone binds the analytes—based on the chemical or biological properties of the surfaces of the capture zone. The surfaces of the zones that contact the liquid sample effectively confine the analytes during the drying step before analysis. With respect to an X3 capture/concentrate chip, the sample presentation device comprises three zones—i.e., a boundary zone, a capture zone, and an analysis zone. The boundary zone is designed to be substantially non-wettable. The capture zone is designed to capture and bind analytes. The analysis zone is designed to be analyte binding resistant. Analytes are transferred between the capture and analysis zones, which is done prior to analysis by one of the various known analytical detection methods. The surface of the analysis zone that contains the liquid sample effectively confines the analytes during the drying step before analysis. The transfer of the liquid sample from the capture zone to the analysis zone may be accomplished by the properties of the surface of the capture zone—i.e., if the capture zone has a lower degree of wettability than the analysis zone, the liquid sample will move from the capture zone to the analysis zone without physical intervention. Alternatively, the capture zone may be designed such that its properties may be changed in response to chemical or physical stimuli (e.g., heat, UV radiation), causing the capture zone to have a lower degree of wettability than the analysis zone, and thus causing the liquid sample to move from the capture zone to the analysis zone.

In another embodiment of the sample presentation devices of the present invention, the sample presentation devices can be combinations of the above-described target and capture chips. In this embodiment, the sample presentation devices are comprised of surfaces having different functionality. These kinds of sample presentation devices may involve the transfer of a liquid sample from one zone to another by mechanical means (e.g., via pipetting) or otherwise (e.g., via the differences in wettability between zones). As an example, a “capture-transfer-concentrate chip,” abbreviated X2-transfer-T3, is a sample presentation device comprised of both an X2 chip comprised of two zones (i.e., a boundary zone and a capture zone), as well as a T3 chip comprised of three zones (i.e., boundary zone, liquid retention zone, and analysis zone). A transfer (mechanical or otherwise) of the analyte occurs between the capture zone of the X2 chip and the liquid retention zone of the T3 chip.

These sample presentation devices may involve more than one “capture zone,” such that the surfaces may exhibit binding affinity to one or more analytes. The ability to bind analytes seriatim as a liquid sample is moved from one zone to another on the surface of the sample presentation devices is a feature of the present invention that facilitates the analysis of many different fractions of a liquid sample without the need to physically separate them using mechanical intervention. Instead, the different wettability properties of the sample presentation devices of the present invention may direct liquid samples to different zones of the devices, in the process leaving behind analytes that bind to different capture zones, and thereby sequentially process a liquid sample.

More specifically, the embodiments of the sample presentation devices that involve combinations of capture zones and liquid retention zones may further be used in a combinatorial manner to isolate, concentrate, purify, and modify analytes in liquid samples prior to their detection. So, for example, a liquid sample may be placed onto a T2 chip such that the analytes in the sample are confined in the analysis zone. That sample may then be transferred to an X3 chip that contains a boundary zone, a capture zone, and an analysis zone. In this example, the capture zone may be designed to bind (and thus remove) lipid moieties from the liquid sample, such that when the sample is applied to the X3 chip, it moves from the boundary zone to the capture zone (which has a higher degree of wettability), the lipid moieties in the sample bind to the surface of the capture zone, and the remaining sample moves to the analysis zone (because it has the highest degree of wettability). In this example, the liquid sample is confined on the T2 chip, and then the lipids are moved on the X3 chip, such that the final sample that is analyzed from the analysis zone is concentrated and purified of lipids. Because the capture zones can be designed to bind a multitude of different analytes, and because various combinations of any of these zones may be used, sample presentation devices having a vast range of purification, concentration, isolation, and modification capabilities (vis-a-vis one or more analytes) can be created.

The mechanism of transfer of liquid samples from one sample presentation device to another may vary. Using the above example, the concentrated sample from T2 may be removed mechanically (e.g., by pipetting) and placed on a separate X3 sample presentation device. Alternatively, the T2 and X3 sample presentation devices may be connected by a zone, the wettability of which may be changed in response to chemical or physical stimuli (e.g., UV radiation), such that the concentrated sample in the analysis zone of the T2 sample presentation device is transferred to the capture zone of the X3 device when the exposure of a zone between them to UV radiation results in a wettability that is higher than the analysis zone of the T2 device but lower than that of the capture zone of the X3 device, such that the sample moves from T2 to X3. Again, with a vast number of surfaces (having different wettability and analyte binding properties) and configurations thereof, sample presentation devices having a vast range of purification, concentration, isolation, and modification capabilities (vis-à-vis one or more analytes) can be created.

The sample presentation devices of the present invention—in each of the embodiments described above—may further provide zones of different wettability having different shapes or patterns (a few examples of which are depicted in the Figures). For example, in one embodiment, a sample presentation device may have zones in the form of concentric circles, with the center zone being the analysis zone, surrounded by the liquid retention zone, surrounded by the boundary zone. Because the zones can be created using a various photo-patterning techniques, and because known photo-patterning techniques provide for tremendous variation in the resulting patterns, there is a vast range of possible shapes, patterns, and configurations of the various zones that can be designed by those of skill in the art. Moreover, the various properties of the different zones of wettability allow for the creation of sample presentation devices capable of directing analytes to single or multiple specified or pre-determined locations on the surfaces (e.g., addressable sites, lanes, or fields). Addressable in this context simply means that the pre-determined site, lane or field can be specified by an automated processing device that works in concert with the sample presentation devices of the present invention such that liquid samples or analytes retained at those specified locations can be processed by an analytical device to measure the analytes of interest. In addition, liquid samples or analytes present at these pre-determined locations may be removed from the sample presentation devices for subsequent handling or manipulation (e.g., modification, purification, concentration, etc.) by another sample presentation device.

The sample presentation devices of the present invention are suitable for the handling of both biological and non-biological liquid samples. They are also suitable for application in a wide range of analyte detection methods, for example, including but not limited to, mass spectrometry, various chromatographic methods, immunofluorescence spectroscopy, and other known analytical methods of detecting and measuring analytes in liquid samples.

Each of the above-described variations is designed to allow for maximum flexibility in design and use of sample presentation devices having enhanced capability to present analytes for detection and analysis over known methods. Thus, the sample presentation devices of the present invention have the capability of directing analytes to an analysis zone designed to enhance high sensitivity detection of analytes. The sample presentation devices of the present invention thus afford improved deposition of analytes.

The sample presentation devices of the present invention may further comprise devices capable of receiving and retaining liquid samples in volumes up to about 100 μL, and at least up to about 70 μL. The sample presentation devices of the present invention may also be utilized as sample positioning devices that directs the deposition of analytes to a surface area measuring less than about 2 millimeter squared (2 mm²) and preferably less than about 1 mm². Directing the deposition of analytes to a surface area measuring less than about 1 mm² may facilitate the improved deposition of analytes with a concomitant increase in both ease of automated data acquisition and sensitivity of detection. Consequently, the sample presentation device of the present invention provides a surface that exhibits substantial utility both with respect to liquid-holding capacity and controlled deposition of analytes. In preferred embodiments, this combination of attributes affords an increase in sensitivity of detection of from about 4-fold to greater than about 100-fold as compared to known sample supports.

In one embodiment, the sample presentation device of the present invention is comprised of a substrate, wherein the surface of the substrate is further comprised of three contiguous zones organized in a concentric arrangement, wherein the central analysis zone is surrounded by a liquid retention zone, and wherein the liquid retention zone is surrounded by a boundary zone. Alternatively, the sample presentation device of the present invention may be comprised of a substrate, wherein the surface is further comprised of three contiguous, zones organized in an adjacent arrangement, wherein some portion of the analysis zone and some portion of the liquid retention zone are contiguous with respect to one another, and wherein those portions of the analysis and liquid retention zones that are not contiguous with respect to one another are surrounded by a common boundary zone.

In an embodiment of the sample presentation devices of the present invention, the surface of the analysis zone has a contact angle of preferably less than about 40°, more preferably less than about 30°, and most preferably less than about 20°. The surface of the analysis zone preferably exhibits minimum affinity or binding with respect to analytes. The surface of the liquid retention zone has a contact angle preferably in the range of about 40° to about 95°, more preferably in the range of about 60° to about 95°, most preferably in the range of about 80° to about 95°, and further preferably exhibits minimum affinity or binding with respect to analytes. The surface of the boundary zone has a contact angle of preferably greater than about 95°, more preferably greater than about 105°, most preferably greater than about 115°, and further preferably exhibits a minimum of wettability with respect to liquid samples.

In another embodiment of the sample presentation devices of the present invention, the contact angle of the analysis zone is at least about 10°, preferably at least about 20°, more preferably at least about 30°, and most preferably at least about 40° lower then the contact angle of the liquid retention zone, wherein the contact angle of the liquid retention zone is preferably at least about 10°, more preferably at least about 15°, and most preferably at least about 20° lower than the contact angle of the boundary zone. In an embodiment of the sample presentation devices of the present invention, the surface area of the liquid retention zone is preferably at least about 4-fold greater, more preferably at least about 10-fold greater, and most preferably at least about 50-fold greater than the surface area of the analysis zone, and the surface area of the analysis zone is preferably less than about 1 mm², is more preferably in the range of from about 0.2 mm² to about 0.8 mm², and is most preferably in the range of from about 0.4 mm² to about 0.6 mm².

The sample presentation devices of the present invention may be further comprised of a substrate, wherein the surface of the substrate may be further comprised of, but not limited to, from 1 to 1536 pairs of analysis zones and liquid retention zones, wherein pairs of analysis zones and liquid retention zones are arranged as either concentric or adjacent pairs, and wherein pairs of analysis and liquid retention zones are surrounded by a common boundary zone. The sample presentation devices comprised of multiple pairs of analysis zones and liquid retention zones is preferably configured in a manner analogous to the standard 96-well, 384-well and 1536-well plates so as to be compatible with standardized multi-well plate processors and laboratory liquid handling robots.

### DESCRIPTION OF THE FIGURES

The descriptions that follow are merely exemplary, supplement the disclosure of the invention set forth elsewhere, and do not limit the scope of the invention.

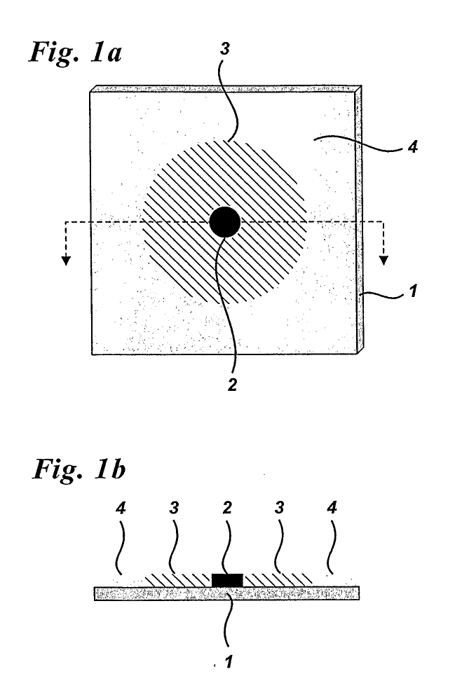

With reference to FIGS. 1a and 1b, the sample presentation device of the present invention is illustrated, showing a substrate 1, wherein the surface of the substrate is further comprised of three contiguous zones organized in a concentric arrangement, wherein the central analysis zone 2 is surrounded by a liquid retention zone 3, and wherein the liquid retention zone 3 is surrounded by a boundary zone 4. The surface of the analysis zone 2 exhibits a contact angle of preferably less than about 40°, more preferably less than about 30°, and most preferably less than about 20°, and further preferably exhibits a minimal binding with respect to analytes. The surface of the liquid retention zone 3 exhibits a contact angle preferably in the range of about 40° to about 95°, more preferably in the range of about 60° to about 95°, most preferably in the range of about 80° to about 95°, and further preferably exhibits minimal binding with respect to analytes. The surface of the boundary zone 4 exhibits a contact angle of preferably greater than about 95°, more preferably greater than about 105°, most preferably greater than about 115°, and further preferably exhibits a minimum of wettability with respect to liquid samples.

With further reference to FIGS. 1a and 1b, a preferred embodiment of the sample presentation device of the present invention is one wherein the contact angle of the analysis zone 2 is preferably at least about 10°, more preferably at least about 20°, more preferably at least about 30°, and most preferably at least about 40° lower than the contact angle of the liquid retention zone 3, wherein the contact angle of the liquid retention zone 3 is preferably at least about 10°, more preferably at least about 15°, and most preferably at least about 20° lower than the contact angle of the boundary zone 4, wherein the surface area of the liquid retention zone 3 is preferably at least about 4-fold greater, more preferably at least about 10-fold greater, and most preferably at least about 50-fold greater than the surface area of the analysis zone 2, and wherein the surface area of the analysis zone 2 is preferably less than about 2 mm², is more preferably in the range of from about 0.2 mm² to about 1.8 mm², and is most preferably in the range of from about 0.4 mm² to about 1.6 mm².

With reference to FIG. 2, the sample presentation device of the present invention is comprised of a substrate 5 wherein the surface is further comprised of 16 concentric pairs of analysis zones 6 and liquid retention zones 7, all of which are surrounded by a common boundary zone 8. In this instance, pairs of target and liquid retention zones are arrayed on 9 mm centers that would allow six of these devices to be combined into the format corresponding to a standard 96-well plate.

With further reference to FIG. 2, a preferred embodiment of the sample presentation device of the present invention is one wherein the contact angle of the analysis zone 6 is preferably at least about 10°, more preferably at least about 20°, more preferably at least about 30°, and most preferably at least about 40° lower then the contact angle of the liquid retention zone 7, wherein the contact angle of the liquid retention zone 7 is preferably at least about 10°, more preferably at least about 15°, and most preferably at least about 20° lower than the contact angle of the boundary zone 8, wherein the surface area of the liquid retention zone 7 is preferably at least about 4-fold greater, more preferably at least about 10-fold greater, and most preferably at least about 50-fold greater than the surface area of the analysis zone 6, and wherein the surface area of the analysis zone 6 is preferably less than about 2 m², is more preferably in the range of from about 0.2 mm² to about 1.8 mm², and is most preferably in the range of from about 0.4 mm² to about 1.6 mm².

It is important to note that neither the analysis zone nor the liquid retention zone must be round in shape as illustrated in FIG. 1a. Both the analysis zone and the liquid retention zone may assume a variety of shapes as may be required to optimize performance of the sample presentation device with respect to a particular application. Additionally, it is important to note that neither the analysis zone nor the liquid retention zone must be concentric with one another as illustrated in FIGS. 1a and 2. Both the analysis zone and the liquid retention zone may be positioned accordingly as may be required to optimize performance of the sample presentation device with respect to a particular application.

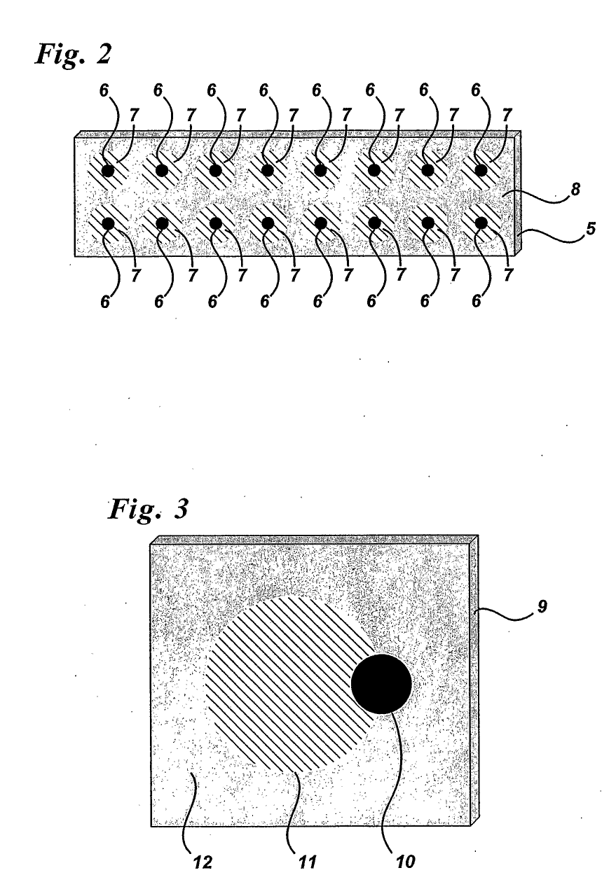

With reference to FIG. 3, the sample presentation device of the present invention is comprised of a substrate 9 having a surface further comprised of three contiguous zones organized in an adjacent arrangement, wherein some portion of the analysis zone 10 and some portion of the liquid retention zone 11 are contiguous with respect to one another, wherein those portions of the analysis zone and liquid retention zone that are not contiguous with respect to one another are surrounded by a common boundary zone 12. The surface of the analysis zone 10 exhibits a contact angle of preferably less than about 40°, more preferably less than about 30°, and most preferably less than about 20°, and further preferably exhibiting minimal binding with respect to analytes. The surface of the liquid retention zone 11 exhibits a contact angle preferably in the range of about 400 to about 95°, more preferably in the range of about 60° to about 95°, most preferably in the range of about 80° to about 95°, and further preferably exhibiting minimal binding with respect to analytes. The surface of the boundary zone 12 exhibits a contact angle of preferably greater than about 95°, more preferably greater than about 105°, most preferably greater than about 115°, and further preferably exhibiting a minimum of wettability with respect to liquid samples.

With further reference to FIG. 3, a preferred embodiment of the sample presentation device of the present invention is one wherein the contact angle of the analysis zone 10 is preferably at least about 10°, more preferably at least about 20°, more preferably at least about 30°, and most preferably at least about 40° lower then the contact angle of the liquid retention zone 11, wherein the contact angle of the liquid retention zone 11 is preferably at least about 10°, more preferably at least about 15°, and most preferably at least about 20° lower than the contact angle of the boundary zone 12, wherein the surface area of the liquid retention zone 11 is preferably at least about 4-fold greater, more preferably at least about 10-fold greater, and most preferably at least about 50-fold greater than the surface area of the analysis zone 10, and wherein the surface area of the analysis zone 10 is preferably less than about 1 mm², is more preferably in the range of from about 0.2 mm² to about 0.8 mm², and is most preferably in the range of from about 0.4 mm² to about 0.6 mm².

It is important to note that neither the analysis zone nor the liquid retention zone must be round in shape as illustrated in FIGS. 1a, 2 and 3. Both the analysis zone and the liquid retention zone may assume a variety of shapes as may be required to optimize performance of the sample presentation device with respect to a particular application.

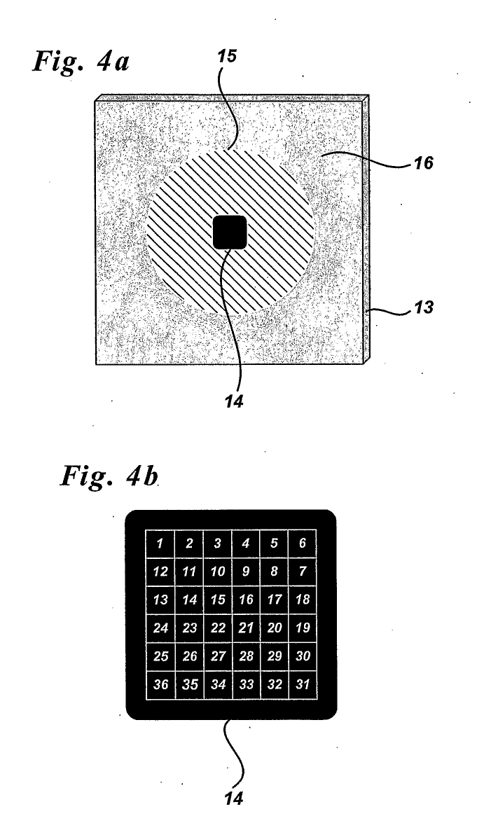

With reference to FIG. 4a, the sample presentation device of the present invention is comprised of a substrate 13 having a surface further comprised of three contiguous zones organized in a concentric arrangement, wherein the central analysis zone 14 is surrounded by a liquid retention zone 15, and wherein the liquid retention zone 15 is surrounded by a boundary zone 16. With reference to FIG. 4b, the shape of the analysis zone 14 (a square) may facilitate automated acquisition of mass spectral data, in that it corresponds in size to a raster of 36 regions.

With reference to FIG. 5, the sample presentation device of the present invention is comprised of a substrate 17 comprised of 96 pairs of analysis zones 18 and liquid retention zones 19, all of which are surrounded by a common boundary zone 20. In this instance, the concentric pairs of zones are arrayed on 9 mm centers that correspond to a standard 96-well plate. The liquid retention zone 19 was been elongated to maximize liquid-holding capacity and minimize the distance between adjacent zones in each row. A serpentine pattern is overlaid on the first two rows of the sample presentation device to indicate the path described by the deposition of a liquid stream of chromatographic eluate during automated fraction collection.

With further reference to FIG. 5, a preferred embodiment of the sample presentation device of the present invention is one wherein the contact angle of the analysis zone 18 is preferably at least about 10°, more preferably at least about 20°, more preferably at least about 30°, and most preferably at least about 40° lower then the contact angle of the liquid retention zone 19, wherein the contact angle of the liquid retention zone 19 is preferably at least about 10°, more preferably at least about 15°, and most preferably at least about 20° lower than the contact angle of the boundary zone 20, wherein the surface area of the liquid retention zone 19 is preferably at least about 4-fold greater, more preferably at least about 10-fold greater, and most preferably at least about 50-fold greater than the surface area of the analysis zone 18, and wherein the surface area of the analysis zone 18 is preferably less than about 2 mm², is more preferably in the range of from about 0.2 mm² to about 1.8 mm², and is most preferably in the range of from about 0.4 mm² to about 1.6 mm².

### Fabrication of Sample Presentation Devices

Still other embodiments of the invention include methods for creating or fabricating the sample presentation devices described above. For example, in an embodiment in which the surfaces are comprised of one or more self-assembled monolayers (SAMs) which form distinct zones depending on differences between the SAMs used, the sample presentation devices of the present invention may comprise various SAM zones that are created by known photo-patterning techniques. Accordingly, the present invention further includes methods of creating sample presentation devices comprised of SAMs using, as one preferred method, photo-patterning techniques.

More generally, the surface of the substrate of the sample presentation device of the present invention is typically modified or patterned by methods known to those of skill in the art. As an example, the substrate's surface can be modified or patterned by means of applying one or more self-assembled monolayers (SAMs), which modify the surface of the substrate of the sample presentation device and whose exposed surfaces may impart particular chemistries to the substrate. Selection of various SAMs, including 1°, 2°, 3°, or 4° compositions, for a particular substrate provides the surface of the substrate with unique surface characteristics and properties. In particular, application of multiple SAMs results in the patterning of the substrate so that it contains a plurality of zones, each zone having different surface characteristics and properties. Methods of patterning the SAMs are known in the art, and include UV photo-patterning, photolithographic patterning, microstamping, electron-beam patterning, and reactive-ion etching.

The zones that are created on the surface of the substrate can be in any shape, with circular shapes being preferred. In addition, the zones can be either continuous or discontinuous with respect to other zones—i.e., the zones can all be contiguous with each other or one or more zones can be discontiguous with one or more other zones. The zones that are created on the surface of the substrate of the sample presentation devices preferably have a plurality of zones of differing wettability with respect to the sample to be analyzed.

As another embodiment of the invention, methods of fabricating a sample presentation device that is capable of precisely positioning analytes so as to facilitate automated data acquisition are provided.

More specifically, approaches to surface patterning, selection of suitable substrates, preparation of self-assembled monolayers as well as other approaches to surface modification are described below. These descriptions are merely exemplary and do not limit the scope of the invention.

The surface of the sample presentation device of the present invention is patterned by one of several approaches which preferably include, but are not limited to: (1) UV-Photopatterning of self-assembled monolayers (SAMs) prepared from alkylthiols on a coinage metal surface; (2) Photolithographic patterning of SAMs prepared from alkylthiols on a coinage metal surface; (3) Microstamping of SAMs prepared from alkylthiols on a coinage metal surface; and (4) Photolithographic patterning of SAMs prepared from alkylsilanes on either a silicon or glass surface; (5) Electron-beam patterning, and (6) Reactive-ion etching. Preferably, the patterning of the sample presentation device surface is achieved either by application of the UV-photopatterning process described in U.S. Pat. No. 5,514,501, or by the microstamping process described in U.S. Pat. No. 5,512,131, both of which are incorporated herein by reference. Alternatively, the patterning of the sample presentation device surface may be achieved by photolithographic patterning processes described in the literature and understood by those skilled in the art.

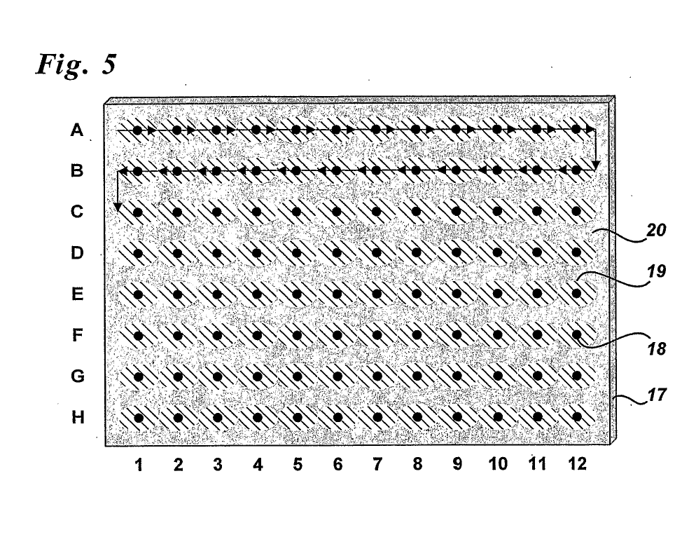

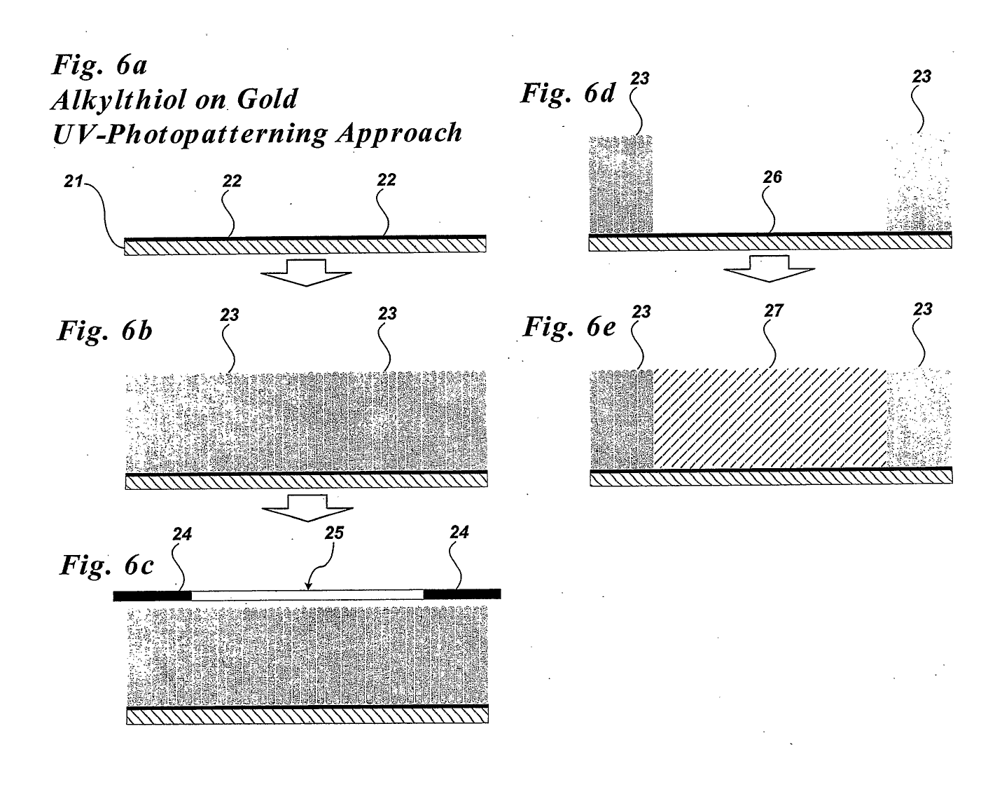

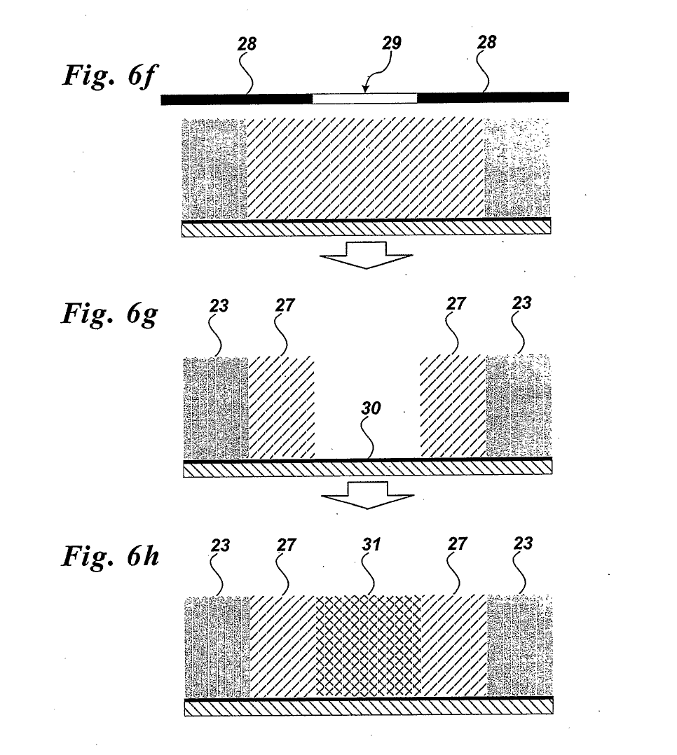

With reference to FIGS. 6a through 6h, the step-wise process for UV-photopatterning of SAMs comprised of alkylthiols on gold is depicted. Initially, a suitable substrate 21 such as a silicon wafer (750 μm) is appropriately cleaned by a combination of wet process and argon plasma etching. An adhesion layer (25-50 nm) of either chromium or titanium and tungsten (9:1) is first applied to the surface of the wafer followed by a thin film (100-1000 nm) of gold 22. Metal deposition is accomplished by a sputtering (vapor deposition) process that has been calibrated with respect to metal deposition (thickness) per unit time. The sputtering process may be undertaken with intact wafers or with individual pieces diced from a wafer.

With reference to FIG. 6b, the first monolayer 23 is assembled on the gold surface by incubation of the substrate in a solution containing from 0.05 to 5 mM alkylthiol in ethanol for a period of from 1 to 24 hours. The surface-modified substrate is then washed with ethanol to remove excess alkylthiol and dried under a stream of nitrogen. The first monolayer 23 is prepared from an alkylthiol which affords a surface that exhibits a contact angle of greater than about 100° and further exhibits a minimum of wettability with respect to liquid samples.

With reference to FIG. 6c, the surface-modified substrate is photo-patterned by exposure to an ultraviolet light source through a first mask 24 in the presence of oxygen so as to oxidize monomers residing within the exposed zone thereby generating monomer sulfonates that exhibit low affinity with respect to the gold surface. The opening in the mask 25 results in the creation of features of size and shape corresponding to the liquid retention zone.

With respect to FIGS. 6d and 6e, subsequent washing of the gold surface removes monomer sulfonates and affords an unmodified region of gold 26. The second monolayer 27 is assembled on the gold surface by incubation of the substrate in a solution containing from 0.05 to 5 mM alkylthiol in ethanol for a period of from 1 to 24 hours. The surface-modified substrate is then washed with ethanol to remove excess alkylthiol and dried under a stream of nitrogen. The second monolayer 27 is prepared from an alkylthiol that affords a surface that exhibits a contact angle in the range of about 40° to about 95° and further affords a surface that exhibits minimal binding with respect to analytes.

With respect to FIG. 6f, the patterned substrate is further photo-patterned by exposure to an ultraviolet light source through a second mask 28 in the presence of oxygen so as to oxidize monomers residing within the exposed zone thereby generating monomer sulfonates that exhibit low affinity with respect to the gold surface. The opening in the mask 29 results in the creation of features of size and shape corresponding to the analysis zone.

With respect to FIGS. 6g and 6h, subsequent washing of the gold surface removes monomer sulfonates and affords an unmodified region of gold 30. The third monolayer 31 is assembled on the gold surface by incubation of the substrate in a solution containing from about 0.05 to about 5 mM alkylthiol in ethanol for a period of from 1 to 24 hours. The surface-modified substrate is then washed with ethanol to remove excess alkylthiol and dried under a stream of nitrogen. The third monolayer 31 is prepared from an alkylthiol that affords a surface that exhibits a contact angle of less than about 40° and further exhibits minimal binding with respect to analytes.

In this manner, the step-wise process for UV-photopatterning of self-assembled monolayers prepared from alkylthiols on gold is exploited to prepare the sample presentation device of the present invention. The above-described process of UV-photopatterning of self-assembled monolayers prepared from alkylthiols on gold is exemplary and the invention is not limited to only the process described.

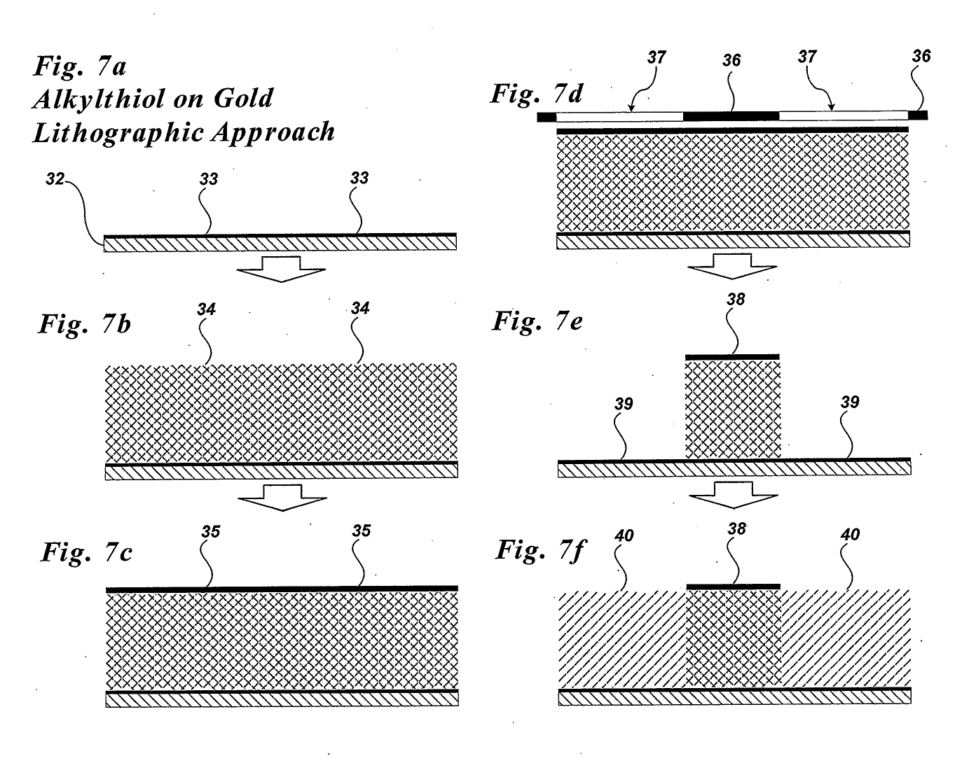

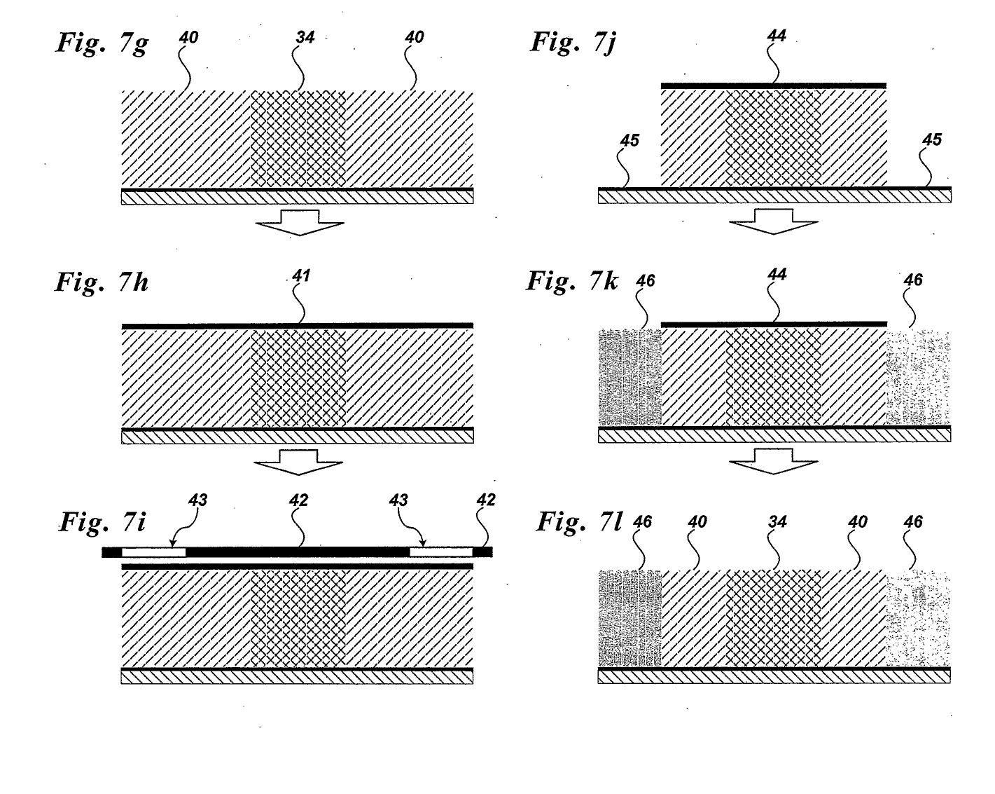

With reference to FIGS. 7a through 7h, the step-wise process for photolithographic patterning of SAMs comprised of alkylthiols on gold is depicted. A suitable substrate 32 such as a silicon wafer is appropriately cleaned and an adhesion layer and a thin film of gold 33 (100-1000 nm) is sputtered thereupon.

With reference to FIG. 7b, the first monolayer 34 is assembled on the gold surface by incubation of the substrate in a solution containing from 0.05 to 5 mM alkylthiol in ethanol for a period of from 1 to 24 hours. The surface-modified substrate is then washed with ethanol to remove excess alkylthiol and dried under a stream of nitrogen. The first monolayer 34 is prepared from an alkylthiol that affords a surface that exhibits a contact angle of less than 40° and further exhibits minimal binding with respect to analytes.

With reference to FIG. 7c, the surface-modified substrate is coated with a photoresist 35 prior to lithography. The resist may be of a negative tone or positive tone. A negative resist results in decreased solubility in the exposed regions of the resist, thus giving a negative image relative to the mask. A positive resist results in increased solubility of the resist in the, exposed regions, thus giving a positive image relative to the mask. The use of a positive resist is depicted. The resist may be applied through a dip-type of process, but is preferable applied using a spin-coater. The manufacturers' recommendations with respect to resist thickness and curing time are used as guidelines.

With reference to FIG. 7d, the surface-modified substrate is photo-patterned by exposure to an ultraviolet light source as required for use in conjunction with the particular resist employed. The photomask 36 may be prepared from a number of commonly employed materials which include, but are not limited to, chromium-on-quartz, Mylar, acetate, and metallic stencils. The opening in the mask 37 results in the creation of features of size and shape corresponding to the analysis zone.

With respect to FIG. 7e, the substrate is initially treated with a commercial solution specific to the resist employed that dissolves the exposed areas of resist while those regions not exposed 38 to the ultraviolet light source remain relatively insoluble. After removal of exposed resist, an oxygen plasma or UV/ozone treatment may be employed to oxidize alkylthiol monomers within the exposed zone thereby generating monomer sulfonates that exhibit low affinity with respect to the gold surface. Subsequent washing of the gold surface removes monomer sulfonates and affords an unmodified region of gold 39.

With reference to FIG. 7f; the second monolayer 40 is assembled on the gold surface by incubation of the substrate in a solution containing from 0.05 to 5 mM alkylthiol in ethanol for a period of from 1 to 24 hours. The substrate is then washed with ethanol to remove excess alkylthiol and dried under a stream of nitrogen. The second monolayer 40 is prepared from an alkylthiol that affords a surface that exhibits a contact angle in the range 40° to 95° and further affords a surface that exhibits minimal binding with respect to analytes.

With respect to FIGS. 7g and 7h, the remaining photoresist 38 is removed by further washing the substrate with one of several organic solvents known to dissolve unexposed resist (e.g. acetone, 1-methyl-2-pyrrolidinone, etc.) and the patterned substrate now comprised of two distinctive zones is coated with fresh photoresist 41 prior to lithography as described above.

With respect to FIGS. 7i and 7j, the patterned substrate is photo-patterned by exposure to an ultraviolet light source through a second photomask 42 as described above. The opening in the mask 43 results in the creation of features of size and shape corresponding to the liquid retention zone. The substrate is initially treated with a commercial solution specific to the resist employed that dissolves the exposed areas of resist while those regions not exposed 44 to the ultraviolet light source remain relatively insoluble. After removal of exposed resist, an oxygen plasma or UV/ozone treatment is employed to oxidize alkylthiol monomers residing within the exposed zone thereby generating monomer sulfonates that exhibit low affinity with respect to the gold surface. Subsequent washing of the gold surface removes monomer sulfonates and affords an unmodified region of gold 45.

With reference to FIGS. 7k and 7l, the third monolayer 46 is assembled on the gold surface by incubation of the substrate in a solution containing from 0.05 to 5 mM alkylthiol in ethanol for a period of from 1 to 24 hours. The substrate is then washed with ethanol to remove excess alkylthiol and dried under a stream of nitrogen. The third monolayer 46 is prepared from an alkylthiol which affords a surface that exhibits a contact angle of greater than 100° and further exhibits a minimum of wettability with respect to liquid samples. Finally, the remaining photoresist 44 is removed by further washing the substrate with one of several organic solvents known to dissolve unexposed resist to afford a patterned surface comprised of three distinctive zones.

In this manner, the step-wise process for photolithographic patterning of SAMs comprised of alkylthiols on gold is exploited to prepare the sample presentation device of the present invention. It should be noted that the sequence of patterning depicted (analysis zone, followed by liquid retention zone, followed by boundary zone) was selected arbitrarily and that the reverse sequence (boundary zone, followed by liquid retention zone, followed by analysis zone) would also prove as suitable as the sequence illustrated. The above-described process of photolithographic patterning of self-assembled monolayers prepared from alkylthiols on gold is exemplary and the invention is not limited to only the process described.

Numerous alkylthiol monomers are suitable for use in preparation of the sample presentation device of the present invention. The synthesis of alkylthiol monomers, their assembly into monolayers, and their classification with respect to the surface tension of the assembled surfaces has been described (Laibinis, P. E.; Palmer, B. J.; Lee, S.-W.; Jennings, G. K. (1998) “The Synthesis of Organothiols and Their Assembly into Monolayers on Gold” in Thin Films, Vol. 24 (Ulman, A., ed.) pp. 1-41, Academic Press, San Diego, Calif.), incorporated herein by reference.

The aforementioned review article has classified terminal moieties associated with alkylthiol SAMs with respect to the surface energy of the assembled surfaces. Moieties which afford highly wettable surfaces and are thus suitable for the preparation of analysis zone monomers include, but are not limited to: CO₂H, B(OH)₂, PO₃H₂, CONH₂ and OH. Each of the aforementioned moieties is reported to afford a surface exhibiting a contact angle of less than about 40°. Generally speaking, moieties that afford highly wettable surfaces are comprised of hydrogen bond acceptors, hydrogen bond donors, and combinations thereof. Terminal moieties which afford surfaces of intermediate wettability and are thus suitable for the preparation of liquid retention zone monomers include, but are not limited to: CN (60°, 10), O₂CCH₃ (63°, 11), CO₂CH₃ (67°, 10), NHCOCH₃ (68°, 11), SCOCH₃ (70°, 11), OCH₃ (74°, 11), CONHCH₃ (76°, 11), NHCOCF₃ (77°, 11) and CO₂CH₂CH₃ (89°, 10). The contact angle associated with the assembled surface and the corresponding alkyl chain length is shown in parenthesis. Generally speaking, moieties which afford intermediately wettable surfaces tend to be comprised of functionalities that participate in dipole-dipole interactions. Terminal moieties which afford minimally wettable surfaces and are thus suitable for the preparation of boundary zone monomers include, but are not limited to: O(CH₂)₂CH₃ (104°, 11), O(CH₂)₃CH₃ (113°, 16), NHCO(CF₂)₇CF₃ (114.5°, 2), O(CH₂)₄CH₃ (115°, 16), O(CH₂)₅CH₃ (115°, 16), OCH₂CF₂CF₃ (118°, 11), and (CF₂)₅CF₃ (118°, 2). The contact angel associated with the assembled surface and the corresponding alkyl chain length is shown in parenthesis. Generally speaking, moieties which afford minimally wettable surfaces tend to be comprised of hydrophobic and oleophobic functionalities.

Preferably, both the target and liquid retention zones of the sample presentation device of the present invention are prepared from monomers that confer protein resistance upon the assembled surface. A number of SAMs prepared from alkylthiols on gold have been specifically characterized with respect to the adsorption of proteins. The most protein resistant of the surfaces thus far reported are those derived from monomers which present oligo(ethylene oxide) (OCH₂CH₂) units. The utility of these surfaces was first described by Prime and Whitesides (Prime, K. L. and Whitesides, G. M. J. Am. Chem. Soc., 1993, 115, 10714-21, incorporated herein by reference). A survey of structure-property relationships of surfaces that resist protein adsorption has appeared (Ostuni, E.; Chapman, R. G.; Holmlin, R. E.; Takayama, S.; Whitesides, G. M. Langmuir, 2001, 17, 5605-5620, incorporated herein by reference). Recently, a number of zwitterionic SAMs have been shown to exhibit good resistance to protein adsorption (Holmlin, R. E.; Chen, X.; Chapman, R. G.; Takayama, S.; Whitesides, G. M. Langmuir, 2001, 17, 2841-50, incorporated herein by reference) and are therefore potentially useful as analysis zones owing to their combination of highly wettable surfaces and good resistance to protein adsorption.

In preferred embodiments, the analysis zone of the sample presentation device of the present invention is prepared from monomers of the General Formula I: HS(CH₂)₁₁—(OCH₂CH₂)ₘOH, wherein m is from 3 to 7. Monomers of this general formula afford surfaces that exhibit contact angles in the range of about 30° to about 38°. Although these surfaces do not exhibit the lowest possible contact angles, they are preferably utilized owing to their superior performance with respect to minimizing the binding of proteins. Furthermore, analysis zone monomers of General Formula I are preferably utilized in conjunction with liquid retention zone monomers that afford surfaces which exhibit contact angles greater than about 60°.

Similarly and preferably, the liquid retention zone of the sample presentation device of the present invention is prepared from monomers of the General Formula II: HS(CH₂)₁₁—(OCH₂CH₂)ₘR, wherein m=3 to 7, and wherein group R is a terminal moiety which influences surface tension and wettability. Preferably but not exclusively, group R is selected from one of OCH₃, OCH₂CN, CO₂CH₃, CONHCH₃, and CO₂CH₂CH₃ moieties. Each of the aforementioned terminal moieties affords a surface that exhibits a contact angle in the range of about 62° to about 89°.

Alternatively and preferably, the liquid retention zone of the sample presentation device of the present invention may be prepared from a monomer of the formula HS(CH₂)₁₁OCH₂C₆H₅. The terminal benzyl moiety (CH₂C₆H₅) exhibits particular utility with respect to samples dissolved in organic solvents and affords a surface that exhibits a contact angle of about 90°.

In preferred embodiments, the boundary zone of the sample presentation device of the present invention is prepared from a monomer which confers a minimum of wettability with respect to liquid samples wherein the analytes are dissolved in aqueous buffers, organic solvents and mixtures thereof. Monomers presenting terminally perfluorinated moieties have been shown to have particular utility in this regard (Naud, C.; Calas, P.; Blancou, H.; Commeyras, A. J. Fluorine Chem., 2000, 104, 173-183, incorporated herein by reference).

A preferred embodiment of the present invention is one wherein the analysis zone is prepared from a monomer of the formula HS(CH₂)₁₁(OCH₂CH₂)₃OH, wherein the liquid retention zone is prepared from a monomer of the formula HS(CH₂)₁₁(OCH₂CH₂)₃OCH₃, and wherein the boundary zone is prepared from a monomer of the formula HS(CH₂)₁₁OCH₂CH₂(CF₂)₅CF₃. This combination of monomers affords a surface wherein the contact angle of the analysis zone, liquid retention zone, and boundary zone are about 38°, 62° and 117°, respectively.

Another preferred embodiment of the present invention is one wherein the analysis zone is prepared from a monomer of the formula HS(CH₂)₁₁(OCH₂CH₂)₃OH, wherein the liquid retention zone is prepared from a monomer of the formula HS(CH₂)₁₁OCH₂C₆H₅, and wherein the boundary zone is prepared from a monomer of the formula HS(CH₂)₁₁OCH₂CH₂(CF₂)₅CF₃. This combination of monomers affords a surface wherein the contact angle of the analysis zone, liquid retention zone, and boundary zone are about 38°, 91° and 117°, respectively.

Mixed (binary) self-assembled monolayers prepared from two alkylthiol monomers have been exploited to precisely control surface contact angle and wettability. (Semal, S.; Bauthier, C.; Voué, M.; Vanden Eynde, J. J.; Gouttebaron, R.; De Coninck, J. J. Phys. Chem. B, 2000, 104, 6225-6232, incorporated herein by reference). Contact angles have been adjusted over a range of greater than 40° by mixing monomers utilized to prepare highly wettable and intermediately wettable surfaces. Preferably, binary SAMs are exploited to prepare either the analysis zone or the liquid retention zone. Alternatively, ternary and quaternary self-assembled monolayers may be exploited to prepare either the analysis zone or the liquid retention zone. Ternary and quaternary SAMs are prepared from binary mixtures of either substituted alkylthiols and hetero-substituted asymmetric alkyl disulfides (i.e., HS(CH₂)₁₁R¹ and R²(CH₂)₁₁S—S(CH₂)₁₁R³) or two hetero-substituted asymmetric alkyl disulfides (i.e., R¹(CH₂)₁₁S—S(CH₂)₁₁R² and R³(CH₂)₁₁S—S(CH₂)₁₁R⁴), respectively.

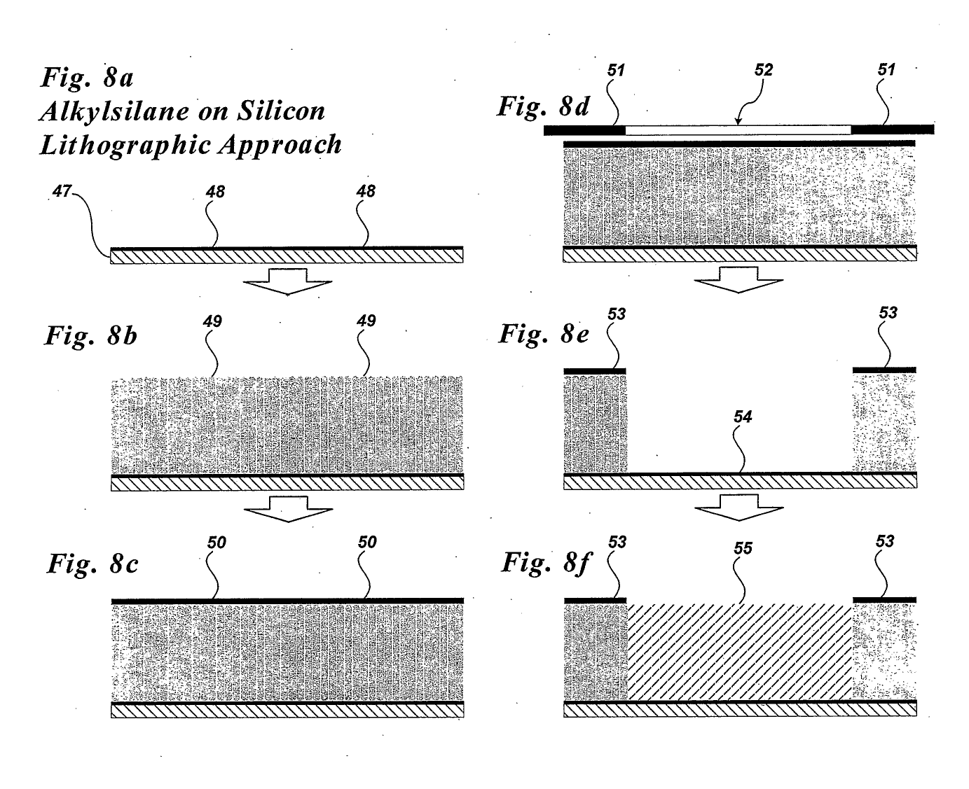

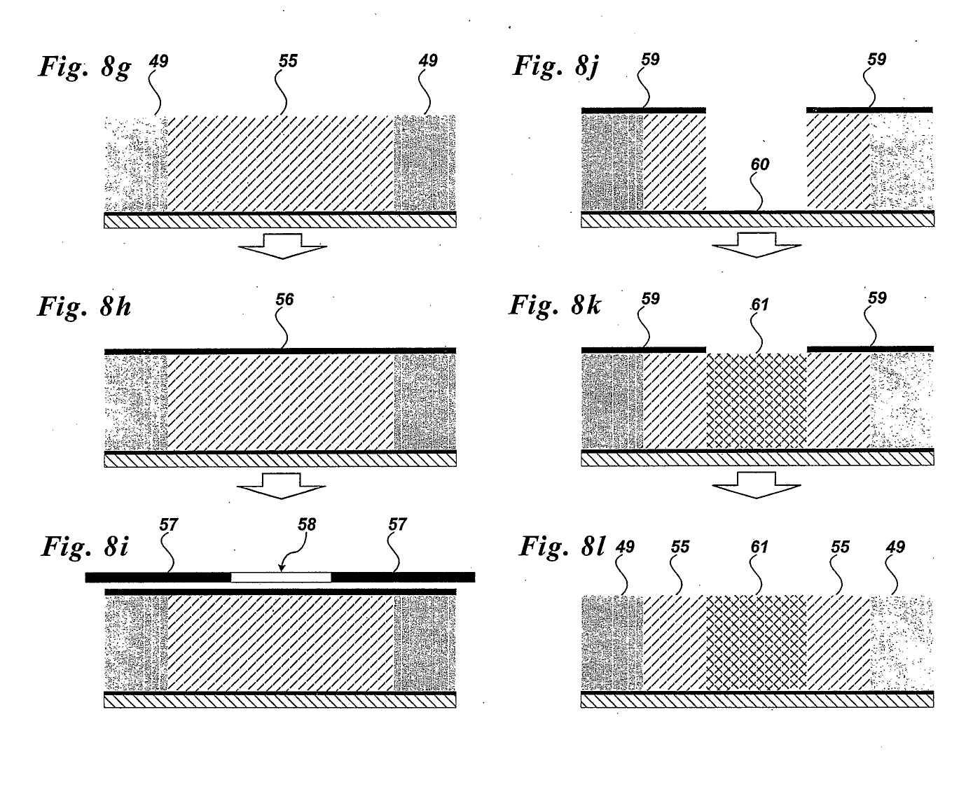

With reference to FIGS. 8a through 8l, the step-wise process for photolithographic patterning of SAMs comprised of alkylsilanes on silicon is depicted. Modification of silicon and glass by reaction with either alkyl dimethylchlorosilanes, alkyl dimethylalkoxysilanes, alkyl trihalosilanes, or alkyl trialkoxysilanes is described in the literature and is understood by those skilled in the art.

With reference to FIG. 8a, a suitable substrate 47 such as a silicon wafer, glass wafer, or metallic substrate with silicon dioxide deposed thereupon is appropriately activated for covalent attachment to an alkylsilane by a process involving removal of surface contaminants followed by oxidation of the surface to generate silanol (Si—OH) moieties. Preferably, the substrate is briefly treated with oxygen plasma, washed with an oxidizing solution (Piranha Solution), and then again treated with oxygen plasma to afford an activated surface 48 that presents an average silanol density approaching 4.9 Si—OH/nm².

With reference to FIG. 8b, following surface activation the first alkylsilane monolayer 49 is assembled on the silicon surface. Silanization may be performed neat, by solution deposition, or by vapor deposition. The first alkylsilane monolayer 49 is preferably prepared from an alkylsilane which affords a surface that exhibits a contact angle of greater than 100° and further exhibits a minimum of wettability with respect to liquid samples.

With reference to FIG. 8c, the silanized substrate is coated with a photoresist 50 prior to lithography. The resist may be of either a negative tone or positive tone. A negative resist results in decreased solubility in exposed regions of the resist, thus giving a negative image relative to the mask. A positive resist results in increased solubility in the exposed regions of the resist, thus giving a positive image relative to the mask. The use of a positive resist is depicted throughout FIG. 6. The resist may be applied through a dip-type of process, but is preferable applied using a spin-coater. The manufacturers' recommendations with respect to resist thickness and curing times should be used as guidelines.

With reference to FIG. 8d, the substrate is photo-patterned by exposure to an ultraviolet light source as required for use in conjunction with the particular resist employed. The photomask 51 may be prepared from a number of commonly employed materials which include, but are not limited to, chromium-on-quartz, Mylar, acetate, and metallic stencils. The opening in the mask 52 results in the creation of features of size and shape corresponding to the liquid retention zone.

With respect to FIGS. 8e and 8f, the substrate is initially treated with a commercial solution specific to the resist employed that dissolves the exposed areas of resist while those regions not exposed 53 to the ultraviolet light source remain relatively insoluble. After removal of exposed resist, an oxygen plasma treatment is employed to activate the surface 54 in preparation for further silanization. The second alkylsilane monolayer 55 is assembled on the activated silicon surface. Silanization may be performed neat, by solution deposition, or by vapor deposition. The second alkylsilane monolayer 55 is prepared from an alkylsilane that affords a surface that exhibits a contact angle in the range of about 40° to about 95° and further affords a surface that exhibits minimal binding with respect to analytes.

With respect of FIGS. 8g and 8h, the remaining photoresist 53 is removed by further washing the substrate with one of several organic solvents known to dissolve unexposed resist (e.g., acetone, 1-methyl-2-pyrrolidinone, etc.) The patterned substrate comprised of two distinctive zones is coated with a photoresist 56 prior to lithography as described above.

With respect to FIGS. 8i and 8j, the patterned substrate is further photo-patterned by exposure to an ultraviolet light source through a photomask 57 as described above. The opening in the mask 58 results in the creation of features of size and shape corresponding to the analysis zone. The substrate is then treated with a commercial solution specific to the resist employed that dissolves the exposed areas of resist while those regions not exposed 59 to the ultraviolet light source remain relatively insoluble. After removal of exposed resist, an oxygen plasma treatment is employed to activate the surface 60 in preparation for further silanization.

With reference to FIGS. 8k and 8l, the third monolayer 61 is assembled on the activated silicon surface. Silanization may be performed neat, by solution deposition, or by vapor deposition. The third alkylsilane monolayer 61 is prepared from an alkylsilane that affords a surface that exhibits a contact angle of less than about 40° and further affords a surface that exhibits minimal binding with respect to analytes. Finally, the remaining photoresist 59 is removed by further washing the substrate with one of several organic solvents known to dissolve unexposed resist to afford a patterned surface comprised of three distinctive zones.

In this manner, the step-wise process for photolithographic patterning of SAMs prepared from alkylsilanes on silicon is exploited to prepare the sample presentation device of the present invention. It should be noted that the sequence of patterning depicted (boundary zone, followed by liquid retention zone, followed by analysis zone) was selected arbitrarily, and that the reverse sequence (analysis zone, followed by liquid retention zone, followed by boundary zone) would also prove as suitable as the sequence illustrated. The above-described process of photolithographic patterning of self-assembled monolayers prepared from alkylsilanes on silicon is exemplary and the invention is not limited to only the process described.

Numerous alkylsilanes are suitable for use in preparation of sample presentation device of the present invention. Alkylsilanes are mostly commercially available and their synthesis and use in surface modification is understood. (Shriver-Lake, L. C. (1998) “Silane-modified surfaces for biomaterial immobilization” Immobilized Biomolecules in Analysis: A Practical Approach (Cass, T. and Ligler, F. S., eds.) Chapter 1, Oxford University Press, Oxford, UK, incorporated herein by reference).

Utilizing an approach that differs somewhat from that outlined above, activated silicon surfaces may be first derivatized with an appropriate alkylsilane having a nucleophilic moiety that is further functionalized by appending a terminal moiety that confers the required wettability. Alternatively, when alkylsilanes with suitable terminal moieties are available, the surface may be modified in a single step. Terminal moieties suitable for use in the preparation of the sample presentation device of the present invention include, but are not limited to, those described above.

In preferred embodiments, the analysis zone of the sample presentation device of the present invention is initially prepared from 3-aminopropyltrimethoxysilane, and then further functionalized to afford an immobilized silane of General Formula III: (XO)₃Si—CH₂CH₂CH₂NHCOCH₂(OCH₂CH₂)ₙOH, wherein X is linkage to either the silicon surface or an adjacent immobilized silane, and wherein n is from 4 to 8. Monomers of General Formula III afford surfaces that exhibit contact angles in the range from about 30° to about 40°. Although these surfaces do not exhibit the lowest possible contact angles, they are preferably utilized owing to their superior performance with respect to minimizing the binding of proteins. Furthermore, analysis zone monomers of General Formula III are preferably utilized in conjunction with liquid retention zone monomers that afford surfaces which exhibit contact angles greater than 60°.

Similarly and preferably, the liquid retention zone of the sample presentation device of the present invention is initially prepared from 3-aminopropyltrimethoxysilane, and then further functionalized to afford an immobilized silane of General Formula IV: (XO)₃SiCH₂CH₂CH₂NHCOCH₂(OCH₂CH₂)ₙR′, wherein X is linkage to either the substrate or an adjacent monomer, wherein n is from 4 to 8, and wherein group R′ is a terminal moiety which influences surface tension and wettability. Preferably but not exclusively, group R′ is selected from one of CH₃, CH₂CN, CH₂CO₂CH₃, CH₂CONHCH₃, and CH₂CO₂CH₂CH₃ moieties. Each of the afore-mentioned terminal moieties affords a surface that exhibits contact angles in the range of about 60° to about 90°.

In preferred embodiments, the boundary zone of the sample presentation device of the present invention is prepared in a single step from an alkylsilane which confers a minimum of wettability with respect to aqueous samples of General Formula V: (CH₃)₂(X′)SiCH₂CH₂—(CF₂)₇CF₃, wherein X′ is a surface reactive moiety.

A variety of alternative surface-modification chemistries and surface patterning approaches may be exploited to prepare the sample presentation devices of the present invention. Polymeric compositions of matter have recently attracted interest with respect to the patterning of protein resistant surfaces. Patterned surfaces initially prepared from either allylthiol or alkylsilane SAMs have been further functionalized by either grafting polymeric compositions to the surface or growing polymeric compositions from the surface (e.g., Husemann, M.; Mecerreyes, D.; Hawker, J. L.; Hedrick, R. S.; Abbott, N. L. Angew. Chem. int. Ed. 1999, 38, 647-649; Shah, R. R.; Merreceyes, D.; Husemann, M.; Rees, I.; Abbott, N. L.; Hawker, C. J.; Hedrick, J. L. Macromolecules 2000, 33, 597-605; and Hyun, J. and Chilkoti, A. Macromolecules 2001, 34, 5644-5652, all incorporated herein by reference). Recently, the first report of surface patterning by adsorption of block copolymers appeared (Deng, T.; Ha, Y.-H.; Cheng, J, Y.; Ross, C. A.; Thomas, E. L. Langmuir, 2002, 18, 6719-6722, incorporated herein by reference). Polymeric thin films grafted to SAMs have been shown to resist the adsorption of proteins to an extent comparable to, or better than, SAMs that present tri(ethyleneglycol) groups (Chapman, R. G.; Ostuni, E.; Liang, M. N.; Meluleni, G.; Kim, E.; Yan, L.; Pier, G.; Warren, H. S.; Whitesides, G. M. Langmuir 2001, 17, 1225-1233, incorporated herein by reference).

It is understood that even the least wettable surfaces may nevertheless retain certain moieties from liquid samples, even if in only a non-specific manner. Such surfaces in fact may contribute to the advantages of the sample presentation devices of the present invention by, for example, enhancing their ability to concentrate analytes by removal of those moieties that are not targets for subsequent analysis. This may be particularly useful in the context of retention of non-biological moieties that might interfere with the analysis of analytes. However, the surfaces of the sample presentation devices are not limited to only this example, but rather may comprise surfaces that bind moieties in regions other than the analysis zone that may be handled or processed separately from the analyte-containing sample. Indeed, any moiety that may be analyzed by analytical biochemical methods may be retained, stored, transported, and subsequently analyzed using the sample presentation devices of the invention. The present invention therefore allows that some retention of moieties in zones other than that having the highest degree of wettability is possible, and that subsequent analysis of those moieties may be desirable. Substantial amounts of the analytes of interest, however, are not typically retained in zones other than those with the highest degree of wettability. Therefore, in the context of the example of analyte analysis by laser desorption spectroscopy, the target analytes retained in the zone of highest wettability are not desorbed from a bound state to the surface of the sample presentation device.

### Uses and Applications of Sample Presentation Devices

The descriptions of various uses and applications of the sample presentation devices of the present invention that follow are merely exemplary and do not limit the scope of the invention.

The sample presentation devices of the present invention find many uses in combination with various analytical techniques and procedures. Thus, the present invention includes methods for using the aforementioned sample presentation devices. More specifically, present invention includes methods of using the sample presentation devices of the present invention to identify the presence of analytes in a sample, and to analyze a plurality of samples, either on a sample presentation device or on a plurality of sample presentation devices.

Virtually any analytical method that permits the detection, identification, or measurement of analytes in a liquid sample can be used in combination with the sample presentation devices of the present invention. Examples of such analytical methods include but are not limited to MALDI-MS or electrospray ionization MS. The sample presentation devices are particularly well suited to us in combination with high throughput analytical measurement techniques, such as, for example, for use in MALDI-MS in which the sample presentation device analysis zones are configured in such fashion as to promote high throughput data acquisition.

The sample presentation devices of the present invention may also be used to manipulate liquid samples, and the analytes contained therein. Based on the differing wettability properties and capture properties that the surfaces of the sample presentation devices may be designed to have, the sample presentation devices may be designed to manipulate, concentrate, position, store, transfer (with and without mechanical intervention), recover (with or without mechanical intervention), analyze, modify or process (via use of analyte modifying reagents on the sample presentation devices), or fractionate liquid samples or the analytes contained therein. Moreover, because the sample presentation devices of the present invention may be designed to accomplish any of these functions in response to chemical or physical stimuli (e.g., heat, UV radiation, pressure, electromagnetic radiation), the sample presentation devices of the present invention may accomplish these functions reversibly or irreversibly, and may further perform various combinations of these functions in response to external forces.

Virtually any liquid sample (and analytes) can be used in connection with the sample presentation devices of the present invention. For example, the present invention can be used to analyze fractions recovered from liquid chromatography. The present invention can be used to analyze enzymatic digests prepared from either protein spots excised from 2D gel electrophoresis or from fractions collected from affinity chromatography (i.e. ICAT). The present invention can also be used to analyze samples recovered from surface plasmon resonance biosensors. The present invention can also be used for 1:1 sample transfer with standard multi-well format robotics and assays. Indeed, the sample presentation devices of the present invention can be used to handle and manipulate liquid samples obtained from virtually any source, whether such samples are the result of laboratory experiment (such as the enzymatic digest and surface plasmon resonance biosensor sample examples identified above), obtained from the environment (such as a water quality sample from a river), or obtained directly from living organisms (such as a human urine sample).

The present invention can also be used for storage of samples for archival purposes or for further analysis. In other words, the detection and analysis of the analytes contained in liquid samples need not occur immediately following transfer of the liquid sample to the analysis zone.

Thus, various embodiments of the present invention provide for sample presentation devices that serve a variety of liquid-handling functions, including but not limited to sample/analyte handling, as well as liquid deposition, retention, transfer, locating and re-locating, and storage. Some examples of these various uses of the sample presentation devices of the present invention are provided.

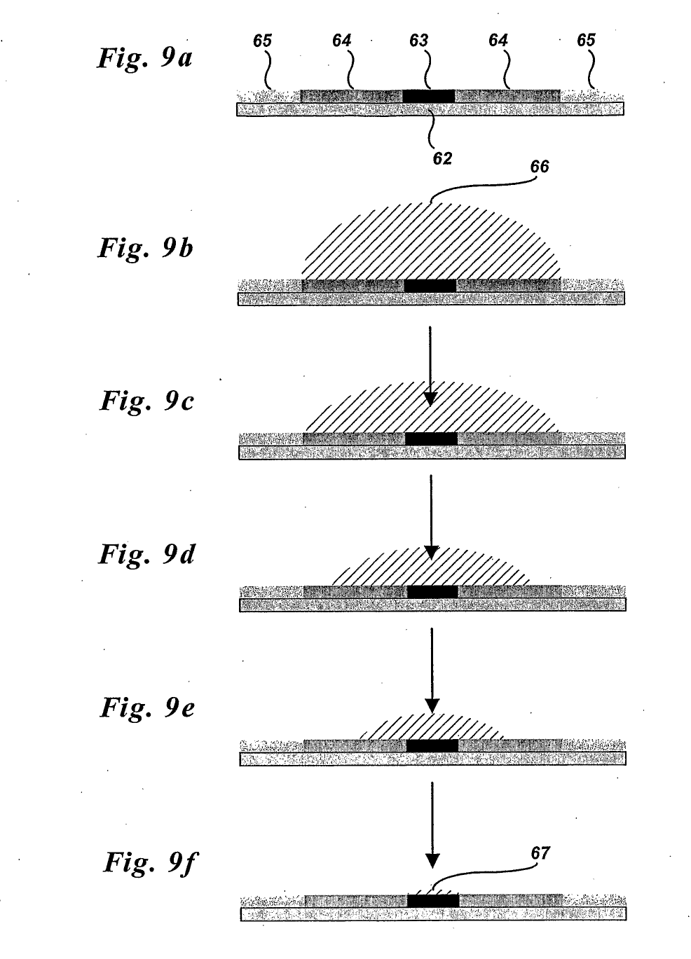

With reference to FIGS. 9a through 9f, various steps in the process of sample drying are illustrated. A cross-sectional view of the sample presentation device of the present invention shows the surface deposited on the substrate 62 comprised of three distinctive zones, wherein the central analysis zone 63 is surrounded by the liquid retention zone 64, and wherein the liquid retention zone 64 is further surrounded by the boundary zone 65.

With reference to FIG. 9b, depositing a liquid sample drop 66 on the surface of the sample presentation device initially results in simultaneous confinement of the sample drop volume to the surface of the analysis zone 63 and the liquid retention zone 64. Sample drop confinement results from the surface tension associated with the limited wettability of the boundary region 65. Upon deposition, the contact angle of the sample drop is approximately equal to that of a drop residing exclusively on the liquid retention zone.

With reference to FIGS. 9c through 9e, as the sample drop dries owing to evaporation, both the radius and the contact angle of the drop recede until the radius of the drop corresponds to that of the analysis zone.

With reference to FIG. 9f, when the radius of the sample drop 67 and that of the analysis zone 63 correspond, the contact angle of the sample drop is found to be approximately equal to that of a drop residing on the analysis zone. As the sample drop continues to dry owing to evaporation, the radius of the sample drop does not further recede, but remains constant as analytes are deposited as a thin film on the surface of the analysis zone. In this manner, aqueous samples of variable volume of up to about 100 μL, deposited on the surface of the sample presentation device, afford upon drying a thin film of analytes confined within an area corresponding to the analysis zone.

For example, the sample presentation device of the present invention with a liquid retention zone having a 3.0 mm diameter (about 7.069 mm² surface area) and a analysis zone having a 0.5 mm diameter (about 0.196 mm² surface area), confines the deposition of analytes to a analysis zone surface area of about 36-fold smaller than the surface area of the liquid retention zone, with an about 36-fold concomitant increase in average surface analyte concentration. Consequently, in principal the sample drop drying process described above would potentially afford an about 36-fold increase in sensitivity.

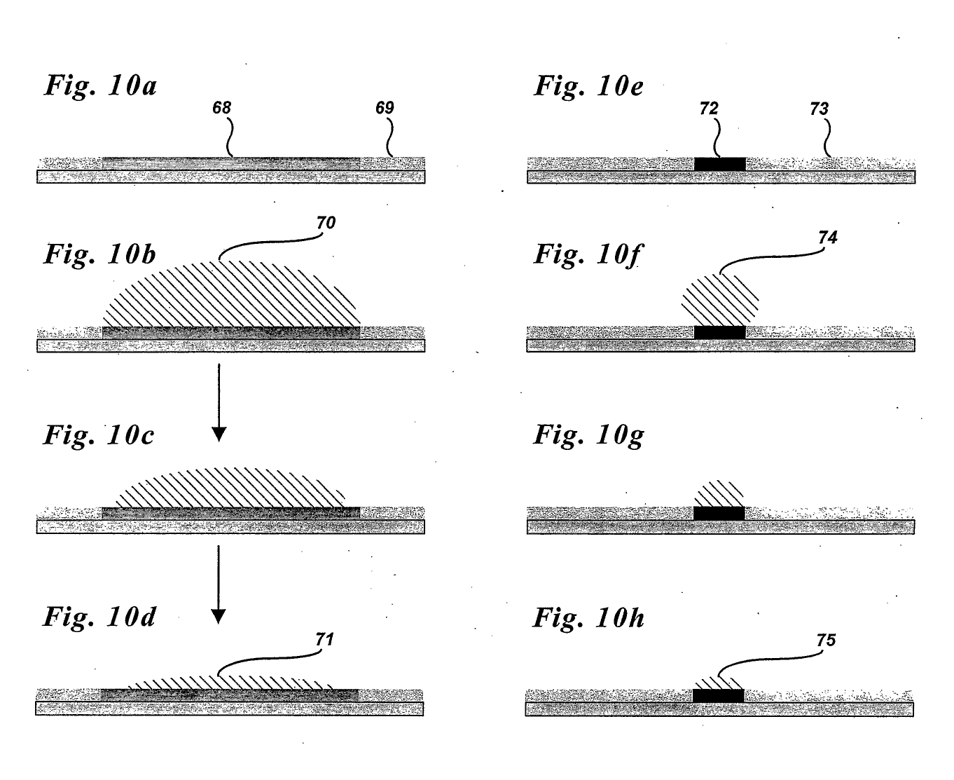

With reference to FIGS. 10a through 10d, in the absence of the analysis zone (only the liquid retention zone 68 and the boundary zone 69 are present) the sample drop 70 dries without a significant reduction in radius resulting in deposition of analytes over much of the surface of the liquid retention zone 71. With reference to FIGS. 10e through 10h, in the absence of the liquid retention zone (only the analysis zone 72 and the boundary zone 73 are present) the volume of the sample drop 74 is limited by the liquid-holding capacity of the analysis zone 72. The sample drop 74 dries without a significant reduction in radius resulting in deposition of analytes over much of the surface of the analysis zone 75.

A significant increase in the sensitivity of detection results from the process described in FIGS. 9b through 9f This phenomenon is best understood with reference to FIGS. 9a through 9d as well as FIGS. 10a through 10d. In the absence of the analysis zone (see FIG. 10a), the average analyte surface concentration per unit area in the liquid retention zone depicted in FIG. 10a, 68 is equal to the total analyte concentration divided by the surface area. In the presence of the analysis zone depicted in FIG. 9a, however, the deposition of analyte is confined to the analysis zone wherein the average analyte surface concentration per unit area is equal to the total analyte concentration divided by the surface area of the analysis zone. Therefore, the presence of the analysis zone 63, depicted in FIG. 9a, affords an increase in average surface concentration of analyte which is equal to the ratio of the surface area of the liquid retention zone, 68, depicted in FIG. 10a, to the surface area of the analysis zone, 63, depicted in FIG. 9a. Since the surface area of the analysis zone is significantly smaller than the surface area of the liquid retention zone, confining analyte deposition to the surface area of the analysis zone results in a significant increase in the average surface concentration of analyte presented to the mass spectrometer with a concomitant increase in sensitivity of detection.

For example, the sample presentation device of the present invention with a liquid retention zone having a 3.0 mm diameter (about 7.069 mm² surface area) and a analysis zone having a 0.5 mm diameter (about 0.196 mm² surface area), confines the deposition of analytes to a analysis zone surface area of about 36-fold smaller than the surface area of the liquid retention zone, with an about 36-fold concomitant increase in average surface analyte concentration. Consequently, in principal the sample drop drying process described above would potentially afford an about 36-fold increase in sensitivity.

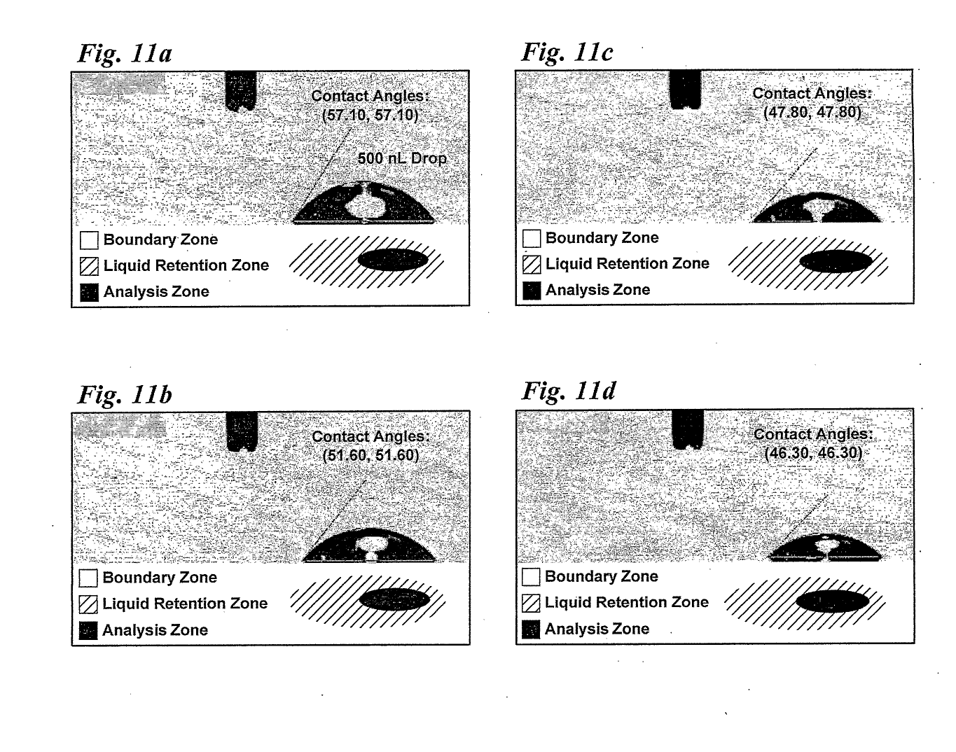

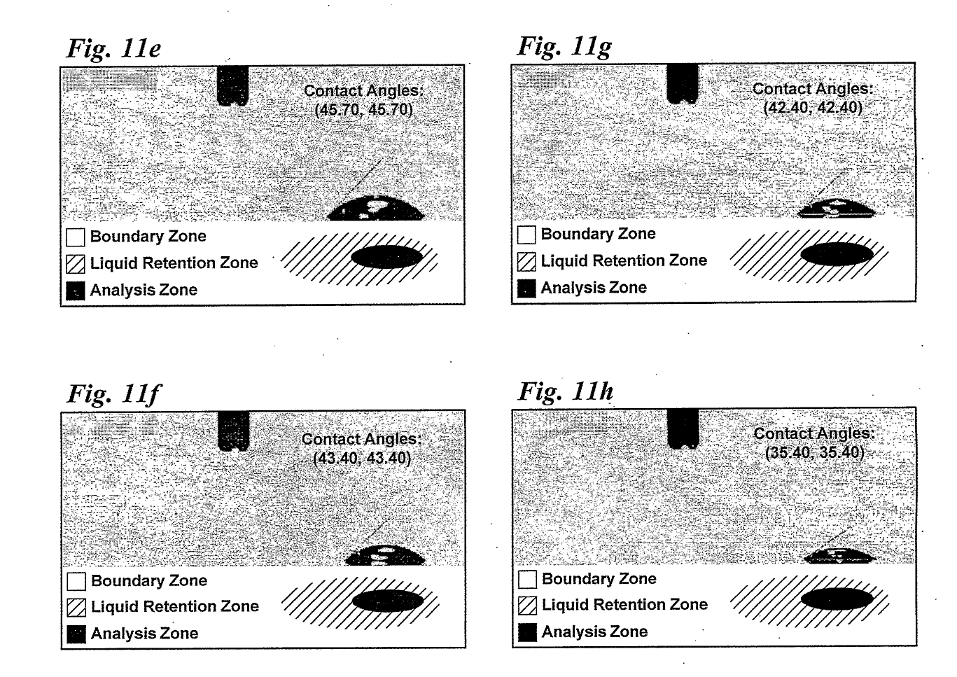

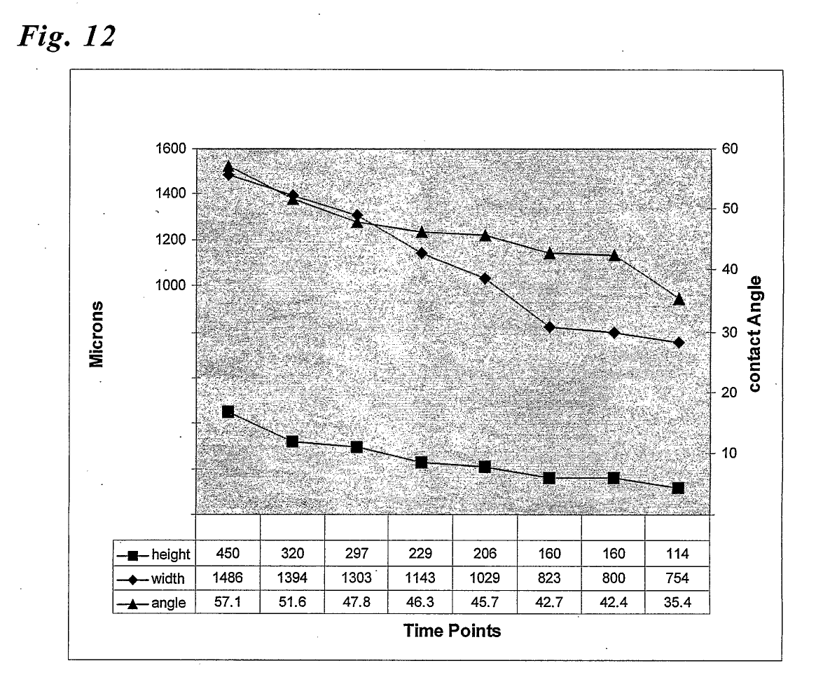

Analyte-confining properties of the analysis zone, which afford an increase in sensitivity of detection, are demonstrated in the video contact angle images shown in FIGS. 11a through 11h. With reference to FIG. 11a, the sample presentation device of the present invention was prepared with a liquid retention zone measuring about 1.6 mm OD and an analysis zone measuring about 0.7 mm OD. To facilitate the observation of the focusing effect, the analysis zone was placed off-center. A drop of water was applied to the surface of the biochip and was observed to rapidly confine itself to the surface area corresponding to the liquid retention zone and the analysis zone. The initial left-side and right-side contact angles were recorded and were both found to be 57.1°, a value which corresponds to that exhibited by a surface prepared from exclusively the liquid retention zone monomer. As the drop dried owing to evaporation (see FIGS. 11b through 11h), both the observed radius and contact angles receded until the radius of the drop corresponded to that of the analysis zone. Furthermore, as the drop dried it was observed that the center of the drop moved to the right so as to allow the drop to center itself over the analysis zone. The left-side and right-side contact angles recorded in FIG. 11h were both found to be 35.4°, a value which corresponds to that exhibited by a surface prepared exclusively from the analysis zone monomer. The drop height, width and contact angle data recorded in conjunction with the acquisition of the images depicted in FIGS. 11a through 11h is summarized graphically in FIG. 12.

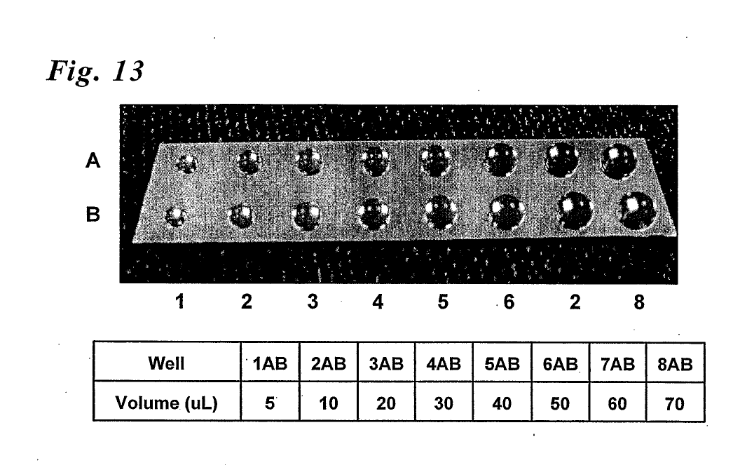

The extraordinary liquid-holding capacity of the liquid retention zone is demonstrated in FIG. 13. An illustration of a 16-site sample presentation device of the present invention shows the retention of sample drop volumes in the range 5 μL to 70 μL. The only factor that appears to significantly limit the sample drop volume is the relative proximity of the adjacent pairs of analysis and liquid retention zones.

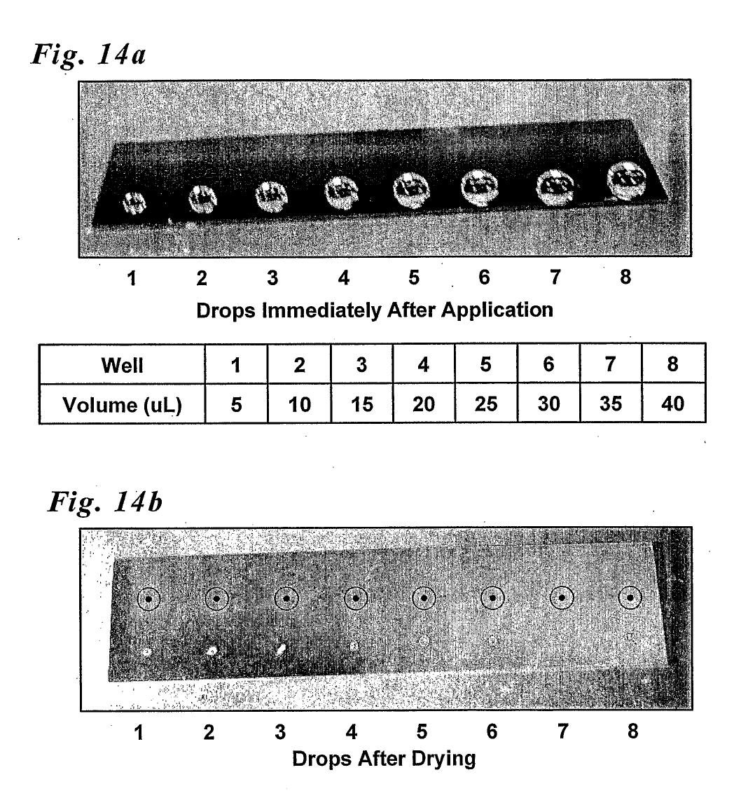

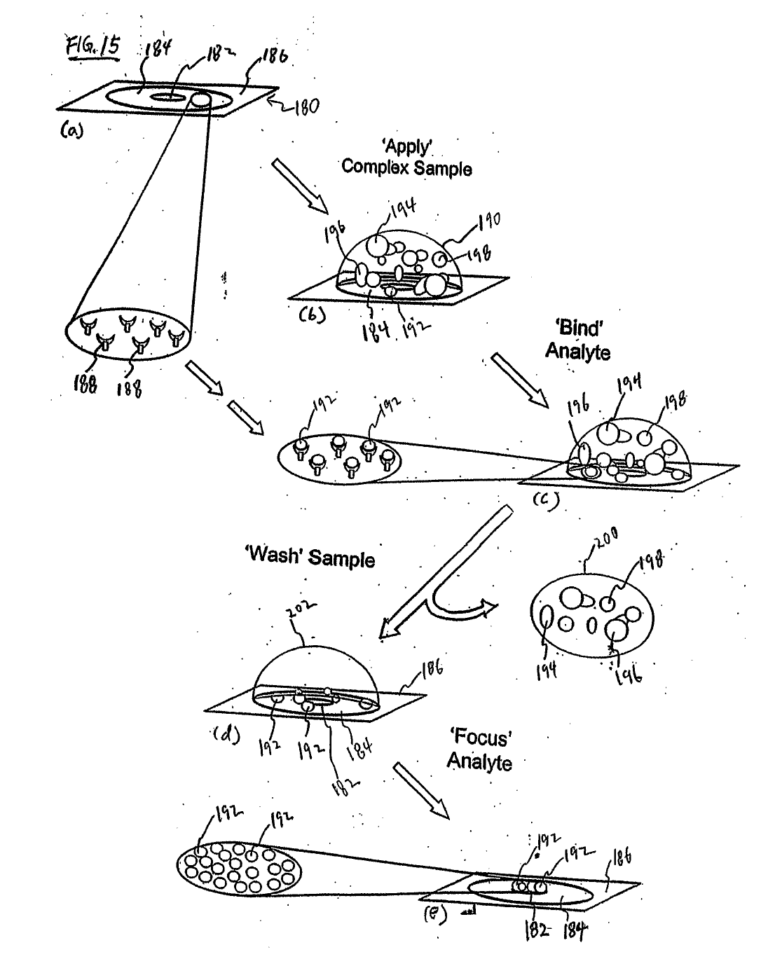

Analyte-confining properties of the analysis zone are further demonstrated in FIGS. 14a and 14b. The first illustration (FIG. 14a) is of a 16-site sample presentation device of the present invention with sample drop volumes in the range 5 μL to 40 μL deposed on the surface of 8 of the 16 sites. Each of the sample drops contained an equivalent amount of a soluble dye. The second illustration (FIG. 12b) is of the same sample presentation device after allowing the sample drops to dry. The dye is now deposed on the surface of the biochip in proximity to the analysis zone. The relative size of the analysis zone and the liquid retention zone is superimposed upon the biochip for comparison purposes. In this instance, an excessive amount of dye was required to afford visible material resulting in the absence of tightly-focused analyte spots.

The sample presentation device of the present invention may be exploited to facilitate high sensitivity mass spectrometric detection of chemical and biological analytes selected from, but not limited to: biological macromolecules such as peptides, proteins, enzymes, enzymes substrates, enzyme substrate analogs, enzyme inhibitors, polynucleotides, oligonucleotides, nucleic acids, carbohydrates, oligosaccharides, polysaccharides, avidin, streptavidin, lectins, pepstatin, protease inhibitors, protein A, agglutinin, heparin, protein G, concanavalin; fragments of biological macromolecules set forth above, such as nucleic acid fragments, peptide fragments, and protein fragments; complexes of biological macromolecules set forth above, such as nucleic acid complexes, protein-DNA complexes, gene transcription complex, gene translation complex, membrane, liposomes, membrane receptors, receptor ligand complexes, signaling pathway complexes, enzyme-substrate, enzyme inhibitors, peptide complexes, protein complexes, carbohydrate complexes, and polysaccharide complexes; and small biological molecules such as amino acids, nucleotides, nucleosides, sugars, steroids, lipids, metal ions, drugs, hormones, amides, amines, carboxylic acids, vitamins and coenzymes, alcohols, aldehydes, ketones, fatty acids, porphyrins, carotenoids, plant growth regulators, phosphate esters and nucleoside diphosphosugars, synthetic small molecules such as pharmaceutically or therapeutically effective agents, monomers, peptide analogs, steroid analogs, inhibitors, mutagens, carcinogens, antimitotic drugs, antibiotics, ionophores, antimetabolites, amino acid analogs, antibacterial agents, transport inhibitors, surface-active agents (surfactants), amine-containing combinatorial libraries, dyes, toxins, biotin, biotinylated compounds, DNA, RNA, lysine, acetylglucosamine, procion red, glutathione, adenosine monophosphate, mitochondrial and chloroplast function inhibitors, electron donors, carriers and acceptors, synthetic substrates and analogs for proteases, substrates and analogs for phosphatases, substrates and analogs for esterases and lipases and protein modification reagents.. Moreover, analytes that may be handled by the sample presentation devices of the present inventions may be non-biological, and include but are not limited to, synthetic polymers, such as oligomers, and copolymers such as polyalkylenes, polyamides, poly(meth)acrylates, polysulfones, polystyrenes, polyethers, polyvinyl ethers, polyvinyl esters, polycarbonates, polyvinyl halides, polysiloxanes, and copolymers of any two or more of the above, as well as oather non-biological analystes such as pesticides.

Analytes may be dissolved in aqueous buffers, organic solvents or mixtures thereof. Buffers are preferably selected from those prepared from volatile constituents including, but not limited to: ammonium acetate, ammonium bicarbonate, ammonium carbonate, ammonium citrate, triethylammonium acetate and triethylammonium carbonate, triethylammonium formate, trimethylammonium acetate, trimethylammonium carbonate and trimethylammonium formate. Aqueous samples containing high concentrations of non-volatile detergents (>0.1%) should be desalted prior to analysis as the presence of detergent may counteract and analyte-confining properties of the analysis zone. Organic solvents are preferably selected from those now to be miscible in aqueous buffers and to promote the solubility of biological analytes including, but not limited to: acetic acid, acetone, acetonitrile, ethanol, N,N-dimethylformamide (DMF), N,N-dimethylsulfoxide (DMSO), formic acid, heptafluorobutyric acid, methanol, N-methylpyrolidone (NMP), 2,2,2-trifluoroethanol and trifluoroacetic acid.

The sample presentation device may be heated during the sample drying process (either on the surface of a heating block, under an infrared lamp or under a stream of hot air) to facilitate the evaporation of high-boiling organic solvent or simply to reduce the time required for sample drying.

Laser desorption time-of-flight mass spectrometry—a preferred analytical method to measure analytes using the sample presentation devices of the present invention requires a material (matrix) to be applied to the surface of the sample presentation device to absorb energy and thereby assist the ionization of analytes. Reagents frequently used as matrices for detection of biological analytes include trans-3,5-dimethoxy-4-hydroxycinlamic acid (sinapinic acid, SA), α-cyano-4-hydroxycinnamic acid (HCCA) and 2,5-dihydroxybenzoic acid (DHBA). Owing to the limited solubility of the aforementioned matrices in water, stock solutions of these reagents often contain 50% to 100% organic solvent. When utilized in conjunction with the sample presentation devices of the present invention, stock solutions containing matrix are added to aqueous samples prior to applying the sample to the surface of the sample presentation device. Alternatively, stock solutions containing matrix may be applied to the surface of the sample presentation device after sample deposition and drying. In this instance, stock solutions containing a high percentage of organic solvent are preferably utilized to minimize dissolving of the analytes deposited on the surface of the analysis zone into the stock solution.

Numerous applications exist for using the sample presentation devices of the present invention. Examples of the types of samples that could be used in the present invention include, but are not limited to, samples that are to be analyzed directly without any processing done before analysis, as well as samples that are to be analyzed indirectly, in that the samples are to be analyzed after some sort of processing has occurred.

Examples of the types of samples that could be used in the present invention that fall into the category of samples that are to be analyzed directly without any processing done before analysis include, but are not limited to, biofluids; tissue and cell extracts and fractions; cells, bacteria, viruses; culture medium; environmental fluids; environmental air sampling; environmental media extracts (soil extracts, solid waste extracts, elution from wipes, elution from air filters); forensic samples; and libraries (combinatorial chemistry, oligonucleotides, peptides, sugars, lipids, cells and components; chromosomes, and viruses and other large protein and nucleoprotein assemblies).

Examples of types of samples that could be used in the present invention that fall into the category of samples that are to be analyzed indirectly, i.e., after some sort of processing has occurred to the samples include, but are not limited to, liquid chromatography (LC) output; gas chromatography (GC) output; elution from gels; digested samples from LC output or gel elutions; mass spectrometry output; elutions from surface plasmon resonance (SPR) or other biosensors; desalting column output; solid-phase extraction output; liquid phase fractionated environmental samples; derivatized samples with respect to any of the above; and other chemical or physical processes and any combinations thereof.

The sample presentation device of the present invention further facilitates the mass spectrometric analysis of biological analytes recovered from fractionation schemes that. exploit either column liquid chromatography or electrophoresis. In particular, utility results from the combination of the liquid-holding capacity of the device (which enables direct collection of chromatographic fractions, samples purified by electrophoresis, samples recovered from sample presentation devices and samples recovered from biosensors without prior sample volume reduction) and the precise positioning of the sample and increased sensitivity of detection (which enables automated data acquisition). The liquid-holding capacity afforded by the sample presentation device of the present invention enables direct collection of fractions recovered from, but not limited to, the following techniques: affinity chromatography, hydrophobic interaction chromatography, ion exchange chromatography, immobilized metal ion affinity chromatography and size exclusion chromatography, as well as fractions recovered from orthogonal separations involving sequential utilization of two or more of the chromatographic approaches enumerated. Furthermore, the availability of the sample presentation device in standard 96-well, 384-well and 1536-well formats enables biochip-based sample collection and processing on multi-well plate processing devices and laboratory liquid handling robots. Consequently, the sample presentation device may be exploited to enable high-throughput mass spectrometric platforms as are needed to support the emergence of proteomics and other important fields of chemistry and biotechnology.

Contemporary protein identification often involves enzymatic digestion of proteins purified either by column liquid chromatography or excised from 2-dimensional electrophoreses gels. Protein digests generally require desalting on reverse phase liquid chromatography (RPLC) or solid-phase extraction (SPE) prior to mass spectrometry. The sample presentation devices of the current invention are suitable for direct collection and subsequent analysis of protein digests desalted by high performance RPLC or SPE.

As a specific example, surface plasmon resonance (SPR) biosensors exploit immobilized proteins to study protein-protein and other biological interactions. Unfortunately, a large volume of eluant is required to recover an analyte from a biosensor and the concentration of analyte in the sample is too low for optimum mass spectrometry. The sample presentation device of the present invention is suitable for direct collection of analytes recovered from biosensor systems; it may be configured to a standard 96-well format so as to be compatible with sample collection devices already integrated into biosensor systems and can be exploited to enable automated sample collection for mass spectrometric analysis, and can concentrate liquid samples of large volumes.

The liquid-holding limitations associated with known mass spectrometer sample presentation devices have prompted the development of various micro-column liquid chromatography approaches involving the use of small pipette tips packed with minute quantities of chromatographic media (e.g., ZipTips®). Micro-column approaches enable the desalting of protein digests with a concomitant reduction in sample volume reported to be sufficient to enable the sample to be applied directly to prior art mass spectrometer devices for retaining samples. The sample presentation devices of the present invention are suitable for direct collection and subsequent analysis of protein digests desalted by micro-column RPLC.

In general, the sample presentation devices of the present invention can be used to accomplish the following with respect to the above-described samples: concentrating; diluting; locating; transporting; storing; presenting for analysis; fractionating; washing; and post-application processing (including digesting, derivatizing, and eluting). It should be understood that this list is not exhaustive and merely provides examples in general terms as to the various applications the sample presentation devices of the present invention can be used.

Once the samples have been applied to the sample presentation devices of the present invention, and the samples have undergone any of the above-identified operations with respect to movement of liquid samples thereon, the following applications can be performed either on the sample presentation device itself or after removal from the device: MALDI-MS; other mass spectrometry techniques; surface plasmon resonance (SPR); fluorescence; atomic force microscopy (AFM); optical spectroscopy; bio- and chemiluminescence; x-ray photoelectron spectroscopy; ellipsometry; electrochemical detection; phosphorescence; and UV, visible and IR spectroscopies. It should be appreciated that this is only a partial list of such applications. It should also be understood that any of the above analyses may be combined and/or serialized, and that where appropriate, these analyses may be performed directly or indirectly upon the analyte(s).

Numerous fields of use are contemplated as being applicable to the sample presentation device of the present invention and include, but are not confined to, such fields as genomics, proteomics, pharmacogenomics, physiomics, toxiomics, metabonomics, drug discovery/drug development/clinical trial monitoring, toxicology, diagnostics, environmental, biosensors, and biological and chemical weapons/bioterrorism. A few specific examples of the applications of the sample presentation devices are described below. The descriptions that follow are merely exemplary and do not limit the scope of the invention.

Genomics: The application of mass spectrometry to genotypic and phenotypic problems has an essential prerequisite of desalting the nucleic acid analyte(s) prior to ionization. Traditionally this desalting is performed before the sample is placed on a MALDI source. In one embodiment, the sample presentation device in an X3 format can accomplish the desalting simultaneously with concentrating the nucleic acid analyte(s). This embodiment is comprised of a reverse phase capture zone and an analyte binding resistant analysis zone. Another embodiment may be comprised of an X4, wherein two capture zones and a single analysis zone would be employed. In a concentric arrangement, the outer capture zone would specifically bind polynucleotide analytes through complementary hybridization with immobilized capture probes; the inner capture zone would perform a desalting function as described above, and the analysis zone presents the analyte for detection. In both of these embodiments, the performance of desalting and presentation for analysis on the same chip increases throughput, minimizes sample loss, and decreases cost.

Drug Discovery/Development/Clinical Trial Monitoring: Many drugs are effective on only a portion of the population. An example of this phenomenon is the drug Herceptin, which is useful for only about 30% of breast cancer patients. In the case of Herceptin, the genetic and protein basis of the sensitivity was integral to the design of the drug, but in most cases the population cannot be divided into likely responders and non-responders prior to expensive and lengthy clinical trials. One of the principal challenges of interpreting such clinical trial results is to understand the biological and/or chemical basis for response and non-response. That knowledge can then be used both for targeting of populations and for further refinement of the drug itself.

One approach to this problem is to obtain profiles (e.g., protein, carbohydrate, lipid) from the patients before, during, and after treatment, and to correlate these profiles with treatment outcome. Several embodiments of the present device can be applied to such studies. Samples (e.g., blood, urine, tissue) obtained from the patients can be subjected to one or more of the pre-processing methods enumerated in the section described above, such as multi-dimensional liquid chromatography, and the fractionated materials produced by that method applied to the device for concentration and presentation for mass spectrometric analysis. Alternatively, samples subjected to minimal processing can be applied to one or more of the present devices with capture zones of known specificity. The analytes are then transferred either to capture zones of complementary specificity before transfer to analysis zones, or directly to analysis zones. In this manner, surfaces with different specificities can be used both in series and in parallel in an automated manner, with the fractionated analytes presented on identical analysis zones for mass spectrometry.

Mass spectrometry provides both profiles (the full mass spectrum) and the opportunity to umambiguously identify specific molecular entities of interest. The mass spectra can then be collected into a database, and multifactorial analysis tools applied to correlate the profiles with patient response. In this way one can discover patterns within the profiles and/or specific molecular entities that enable: prediction of response to therapy; monitoring of response to therapy; and identification of molecular entities that affect response to therapy, thus allowing increasingly sophisticated drug design.

This area of scientific inquiry, like the others described herein, is dependent in large measure on the ability to measure analytes in liquid solution. The sample presentation devices of the present invention, and their uses described herein, represent an important tool that can be used to conduct further study.

Environmental: Analyzing environmental samples for the presence of contaminants is a worldwide effort. Among the particular problems faced by such studies are the low concentrations of analytes and the diversity of samples that must be studied, as contaminants may be present in gaseous, liquid, and solid materials. In general, such analyses involve collection, extraction, derivatization, fractionation, and detection steps.

The present devices may be applied in a number of ways to the analysis of environmental samples. These examples are representative, but by no means complete. Devices with capture zones can be used for direct collection of analytes from gaseous or liquid media. For example, capture of hydrophobic pesticide residues from aqueous solutions by a hydrophobic surface may replace liquid/liquid extractions, which can be time-consuming and generate hazardous waste. The collected material can then be transferred directly to analysis zones, fractionated by serial or parallel transfer to capture zones of complementary specificity prior to transfer to analysis zones, or transferred from the device to enable analysis by one or more of the techniques entunerated in the sections described above. Mass spectrometry is generally used for identification of pesticide residues, but other techniques such as immunoassay may be applied. The present devices can also be used as previously described to present and/or fractionate materials resulting from any of the steps of environmental analysis listed above. The present/devices can be used as a platform to derivatize analytes and present them for analysis in altered form. For example, silyl- and/or acetyl-moieties may be added to pesticides immobilized on the device to enable unambiguous identification of molecular structure.

Biological and Chemical Weapons/Bioterrorism: The United States government is confronted with the need for platforms and analytical techniques to facilitate the detection of chemical and biological agents in both military and civil scenarios. Challenges for biowarfare detection include sample collection and distinguishing between innocuous versus toxic organisms. The current battlefield technique for bio agents utilizes pyrolysis to convert biological compounds to small, easily detectable molecules by MS. A technique relying on peptide biomarkers is largely anticipated, since it would be more specific than current methods. Tests on individuals to determine potential exposure to warfare agents should involve breath tests or blood drawing techniques. Stand-alone biosensors as alerting devices are also of great interest for use in public places or in the battlefield. All these methods present challenges in sample collection, pre-treatment, and presentation of samples to detectors by robotics or other remote means. Techniques that can store, manipulate, concentrate or purify samples or those that can be coupled to aerosol impactors currently used have the potential of attracting the interest of defense agencies. The present devices can be applied to biowarfare/bioterror detection in a manner similar to that described for environmental samples. In addition, devices with custom capture zones can be designed to collect microorganisms of interest from environmental or biofluid samples, allow processing of the cells (or viruses) to release key markers, and present those markers for detection.

The following examples provide additional detail about the composition, manufacture, and use of the sample presentation devices of the present invention, but are exemplary only and do not in any way limit the scope of the present invention.

### EXAMPLE I

### Preparation of 11-(3,3,4,4,5,5,6,6,7,7,8,8,8-Tridecafluorooctyloxy)undec-1-ene (1)

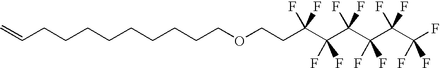

An amber shell vial (40 mL) was charged with 3.0 mL of 1H,1H,2H,2H-perfluorooctanol (13.7 mmol) and to this was added 1.4 mL of 50% aqueous potassium hydroxide (13.7 mmol). The solution was warmed to 80° C., stirred for 30 minutes and 3.3 mL of 11-bromoundec-1-ene (1.5 mmol) added. The reaction was maintained at 80° C. for 52 hours until TLC analysis (hexane) showed the starting material was consumed. The product was allowed to cool to room temperature, added to 100 mL ethyl acetate and extracted with water (2×50 mL) and brine (1×50 mL). The ethyl acetate extract was dried over magnesitun sulfate, filtered and the solvent evaporated in vacuo to afford an oily residue. The residue was purified on a silica gel flash column (50×300 mm, 0% ethyl acetate/hexane followed by 10% ethyl acetate/hexane). Fractions containing the desired product were combined and the solvent evaporated to afford 4.52 g (64%) of 1 as a colorless oil. ¹H NMR (400 MHz, CDCl₃): δ 5.80 (m, 1H), 4.95 (m, 2H), 3.69 (t, J=6.8 Hz, 2H), 3.43 (t, J=6.8 Hz, 2H), 2.39 (m, 2H), 2.03 (m, 2H), 1.55 (m, 2H), 1.36 (m, 2H), 1.27 (broad m, 10H).

### EXAMPLE II

### Preparation of Thioacetic Acid S-[11-(3,3,4,4,5,5,6,6,7,7,8,8,8-Tridecafluorooctyloxy)undecyl]Ester (2)

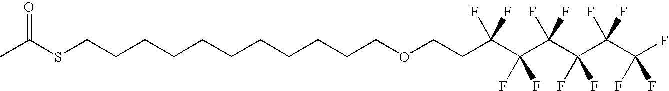

A dry round bottom flask (100 mL) was charged with 1.0 g of 1 (1.9 mmol) under argon and 10 mL of dry methanol added. To the resulting solution was added 426 μL of thiolacetic acid (6.0 mmol) followed by 52 mg of 2,2′-azobis(2-methylpropionamidine)dihydrochloride (0.2 mmol). The reaction was shrouded in a foil tent and exposed to light from a low pressure mercury lamp. After 4 hours, TLC analysis (5% ethyl acetate/hexane) revealed that the starting material had been consumed. The solvent was evaporated in vacuo to give an oily residue. The residue was purified on a silica gel flash column (40×300 mm, 0% ethyl acetate/hexane followed by 5% ethyl acetate/hexane). Fractions containing the desired product were combined and the solvent evaporated to afford 856 mg (76%) of 2 as a colorless oil. ¹H NMR (400 MHz, CDCl₃): δ 3.69 (t, J=6.8 Hz, 2H), 3.43 (t, J=6.8 Hz, 2H), 2.39 (m, 2H), 2.31 (s, 3H), 1.55 (m, 2H), 1.33 (m, 2H), 1.25 (broad m, 10H)

### EXAMPLE III

### Preparation of 11-(3,3,4,4,5,5,6,6,7,7,8,8,8-Tridecafluorooctyloxy)undecane-1-thiol (3)

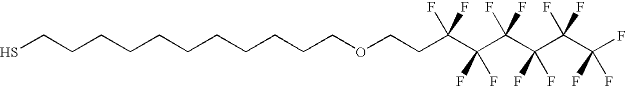

An amber shell vial (20 mL) was fitted with a Teflon-lined silicon septum, charged with 850 mg of 2 (1.1 mmol) and 5 mL of 3N methanolic hydrogen chloride (15 mmol) added. The resulting solution was warmed to 40° C. for 4 hours. The solvent was removed to afford 782 mg (98%) of 3 as a colorless oil. ¹H NMR (400 MHz, CDCl₃): δ 3.69 (t, J=6.8 Hz, 2H), 3.43 (t, J=6.6 Hz, 2H), 2.51 (dd, J=7.3, 7.6 Hz, 2H), 2.39 (m, 2H), 1.58 (m, 4H), 1.32 (t, J=8.0 Hz, 1H), 1.25 (broad m, 12H).

### EXAMPLE IV

### Preparation of 11-{2-[2-(2-Methoxyethoxy)ethoxy]ethoxy}undec-1-ene (4)

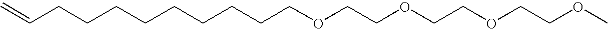

A round bottom flask (200 mL) was charged with 27.4 mL of triethyleneglycol monomethyl ether (171 mmol) and 9.1 mL of 50% aqueous sodium hydroxide (114 mmol) added. The pale yellow solution was warmed to 80° C, stirred for 30 minutes and 26.6 mL of 11-bromoundec-1-ene (114 mmol) was added dropwise. The reaction was maintained at 80° C. for 7.5 hours until TLC analysis (100% ethyl acetate) showed the starting material to be consumed. The product was cooled to room temperature, diluted into 50 mL of water and extracted with hexanes (3×50 mL). The hexanes extracts were combined, dried over magnesium sulfate, filtered and the solvent evaporated in vacuo to afford 20 g (56%) of 4 as a clear, colorless oil. ¹H NMR (400 MHz, CDCl₃): β 5.81 (m, 1H), 4.96 (m, 2H), 3.68-3.56 (m, 12H), 3.44 (t, J=6.8 Hz, 2H), 3.38 (s, 3H), 2.04 (m, 2H), 1.57 (m, 2H), 1.36 (m, 2H), 1.27 (broad s, 10H).

### EXAMPLE V

### Preparation of Thioacetic acid S-(11-{2-[2-(2-methoxyethoxy)ethoxy]ethoxy}undecyl)ester (5)

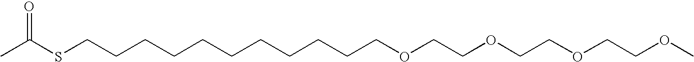

A dry round bottom flask (200 μL) was charged with 5.0 g of 4 (15.8 mmol) under argon and 10 mL of dry methanol was added. To this was added 3.6 mL of thiolacetic acid (50 mmol) followed by 434 mg of 2,2′-azobis(2-methylpropionamidine)dihydrochloride (1.6 mmol). The reaction was shrouded in a foil tent and exposed to light from a low pressure mercury lamp. After 15.5 hours, TLC analysis (ethyl acetate/hexane, 1:3) revealed the starting material had been consumed. The solvent was evaporated in vacuo to give a residue with a strong sulfur-like odor. The residue was purified on a silica gel flash column (40×300 mm, 30% ethyl acetate/hexane, and 50% ethyl acetate/hexane). Fractions containing the desired product were combined and the solvent was evaporated to afford 5.83 g (94%) of 5 as a colorless oil. ¹H NMR (400 MHz, CDCl₃): δ 3.67-3.54 (m, 12H), 3.44 (t, J=7.2 Hz, 2H), 3.38 (s, 3H), 2.86 (t, J=7.2 Hz, 2H), 2.32 (s, 3H), 1.57 (m, 4H), 1.36-1.26 (broad m, 14H).

### EXAMPLE VI

### Preparation of 11-{2-[2-(2-Methoxyethoxy)ethoxy]ethoxy}undecane-1-thiol (6)

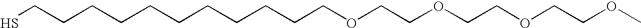

An amber shell vial (20 mL) fitted with a Teflon-lined silicon septum was charged with 5.0 g of 5 (12.7 mmol) and 7 mL of 3N methanolic hydrogen chloride (21 mmol) was added. The solution was warmed to 40° C. for 6 hours. The solvent was then evaporated in vacuo to afford 4.40 g (98%) of 6 as a colorless waxy gel. ¹H NMR (400 MHz, CDCl₃): δ 3.67-3.54 (m, 12H), 3.44 (t, J=6.8 Hz, 2H), 3.37 (s, 3H), 2.51 (dd, J=7.3, 8.0 Hz, 2H), 1.57 (m, 4H), 1.32 (t, J=7.6 Hz, 1H), 1.26 (broad m, 14H).

### EXAMPLE VII

### Preparation of 2-[2-(2-Undec-10-enyloxyethoxy)ethoxy]ethanol (7)

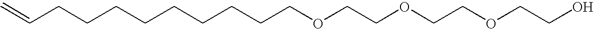

A round bottom flask (250 mL) was charged with 67.0 mL of triethyleneglycol (0.5 mol) and 8.0 mL of 50% aqueous sodium hydroxide (8 mL, 0.1 mol) was added. The solution was warmed to 100° C., stirred for 30 minutes and 22.0 mL of 11-bromoundec-1-ene (0.1 mol) were added dropwise to give a dark yellow solution which produced a precipitate of sodium bromide. The reaction was maintained at 100° C. for 2.5 hours until TLC analysis (methanol/ethyl acetate/hexane, 1:1:8) revealed that the starting material to be consumed. The reaction was cooled to room temperature, diluted into 300 mL of water and extracted with hexanes (3×100 mL). The organic extracts were combined, washed with brine (50 mL), dried over magnesium sulfate and filtered. The solvent evaporated in vacuo to give an oily residue. The residue was purified on a silica gel flash column (50×400 mm, methanol/ethyl acetate/hexane 5:5:90). Fractions containing the desired product were combined and the solvent was evaporated to give 20.8 g (69%) of 7 as a clear oil. ¹H NMR (400 MHz, CDCl₃): δ 5.78 (m, 1H), 4.93 (m, 2H), 3.72-3.55 (m, 12H), 3.42 (t, J=7.2 Hz, 2H), 2.64 (t, J=5.6 Hz, 1H), 2.01 (m, 2H), 1.54 (m, 2H), 1.34 (m, 2H), 1.25 (broad s, 10H).

### EXAMPLE VIII

### Preparation of Thioacetic acid S-(11-{2-[2-(2-hydroxyethoxy)ethoxy]ethoxy}undecyl)ester (8)

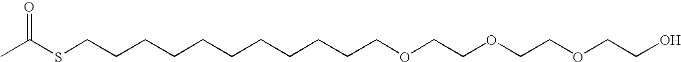

A dry round bottom flask (100 mL) was charged with 2.0 g of 7 (6.6 mmol) under argon and 10 mL of dry methanol added. To this was added 2.85 mL of thiolacetic acid (40 mmol) followed by 271 mg of 2,2′-azobis(2-methylpropionamidine)dihydrochloride (1.0 mmol). The reaction was shrouded in a foil tent and exposed to with light from a low pressure mercury lamp. After 6 hours, TLC analysis (methanol/ethyl acetate/hexane, 1:1:8) revealed that the starting material had been consumed. The solvent was evaporated in vacuo to give yellow oil. The oil was purified on a silica gel flash column (50×300 mm, methanol/ethyl acetate/hexane, 1:1:8). Fractions containing the desired product were combined and the solvent was evaporated to afford 2.44 g (98%) of 8 as a light yellow oil. ¹H NMR (400 MHz, CDCl₃): δ 3.71-3.54 (m, 12H), 3.42 (t, J=6.6 Hz, 2H), 2.83 (t, J=7.2 Hz, 2H), 2.66 (broad s, 1H), 2.29 (s, 3H), 1.52 (m, 4H), 1.36-1.23 (broad m, 14H).

### EXAMPLE IX

### Preparation of 2-{2-[2-(11-Mercaptoundecyloxy)ethoxy]ethoxy}ethanol (9)

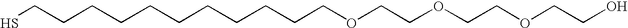

An amber shell vial (20 mL) was fitted with a Teflon-lined silicon septum, charged with 2.40 g of 8 (6.4 mmol) and 5.0 mL of 3N methanolic hydrogen chloride (15 mmol) added. The resulting solution was warmed to 40° C for 4 hours. The solvent was then evaporated in vacuo to afford 2.05 g (95%) of 9 as a colorless waxy gel. ¹H NMR (400 MHz, CDCl₃): δ 3.72-3.55 (m, 12H), 3.43 (t, J=6.8 Hz, 2H), 2.71 (broad s, 1H), 2.50 (dd, J=7.6, 7.4 Hz, 2H), 1.62-1.52 (m, 4H), 1.31 (t, J=7.6 Hz, 1H), 1.26 (broad m, 14H).

### EXAMPLE X

### Preparation of Undec-10-enyl-oxymethylbenzene (10)

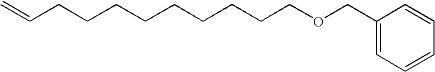

A dry round bottom flask (100 mL) was charged with 5.0 g of undec-10-en-1-ol (29.4 mmol) under argon and 25 mL of dry N,N-dimethylformamide was added. The resulting solution was cooled to 0° C. and 2.16 g of 60% sodium hydride in mineral oil (45 mmol) was added in one portion. The frothing mixture was stirred under argon at 0° C for 30 minutes. To the chilled, stirred solution was added dropwise 7.7 g of bromomethylbenzene (45 mmol) in 5 mL of dry N,N-dimethylformamide and the reaction was allowed to warm to room temperature while stirring for 3 hours. The reaction was quenched by the slow addition of 100 mL of ethyl acetate, extracted with 1N hydrochloric acid (2×50 mL) and brine (1×50 ml). The organic layer was dried over magnesium sulfate, filtered and the solvent evaporated to give an oily residue (9.5 g). The residue was purified on a silica gel flash column (50×300 mm, 94:5:1 hexane/toluene/ethyl acetate) and the fractions containing the desired product were combined. Finally, the solvent was evaporated in vacuo to afford 7.1 g (93%) of 10 as a colorless oil. ¹H NMR (400 MHz, CDCl₃): δ 7.32 (d, 4H), 7.28 (m, 1H), 5.81 (m, 1H), 4.95 (m, 2H), 4.49 (s, 2H), 3.46 (t, 2H), 2.03 (m, 2H), 1.61 (m, 2H), 1.35 (broad m, 4H), 1.24 (broad s, 10H).

### EXAMPLE XI

### Preparation of Thioacetic Acid S-(11-Benzyloxyundecyl)ester (11)

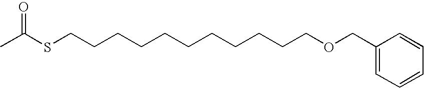

A jacketed photo-reaction vessel (250 mL) was first charged with 5.0 g of 10 (19.2 mmol) and 0.520 g of 2,2′-azobis(2-methylpropionamidine)dihydrochloride (1.92 mmol). The vessel was sealed, evacuated and back-flushed with argon (several cycles). While under argon, 60 mL of anhydrous methanol and 0.520 g of thioacetic acid (92 mmol) were injected into the reaction vessel and the contents of the vessel were stirred. The vessel was again evacuated and back-flushed with argon (several cycles). The UV lamp was activated and the mixture irradiated under argon with constant stirring for 3 hours. The reaction was continually cooled (water jacket) and the temperature maintained below 38° C. during the photo-reaction process. The reaction vessel was allowed to cool to room temperature and the solvent was evaporated to give pale yellow oil (10.8 g). The oil was purified on a silica gel flash column (50×300 mm, 98:2 hexane/ethyl acetate) and the fractions containing the desired product were combined. Finally, the solvent was removed in vacuo to afford 5.0 g (77%) of 11 as a colorless oil. ¹H NMR (400 MHz, CDCl₃): δ 7.32 (d, 4H), 7.28 (m, 1H), 4.49 (s, 2H), 3.46 (t, 2H), 2.86(t, 2H), 2.31(s, 3H), 1.50-1.66 (m, 4H), 1.20-1.40 (broad m, 14H).

### EXAMPLE XII

### Preparation of 11-Benzyloxyundecane-1-thiol (12)

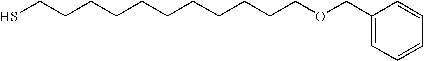

An amber shell vial (40 mL) was fitted with a Teflon-lined silicon septum, charged with 3.04 g of 11 (9.03 mmol) followed by 2 mL of dichloromethane, 1 mL of hexane, and 12 mL of 4.9 N ethanolic hydrogen chloride. The resulting solution was warmed to 40° C. for 4.5 hours. The solvent was then evaporated in vacuo to afford a colorless oily residue (2.8 g). The residue was purified on a silica gel flash column (25×450 mm, 9:1 hexane/chloroform) and the fractions containing the desired product were then combined. The solvent was evaporated in vacuo to give 2.5 g (94%) of 12 as a colorless oil. ¹H NMR (400 MHz, CDCl3): δ 7.32 (d, 4H), 7.28 (m, 1H), 4.49 (s, 2H), 3.46 (t, 2H), 2.51(q, 2H), 1.55-1.65 (m, 4H), 1.20-1.40 (broad m, broad t, 15H).

### EXAMPLE XIII

### Preparation of Self-Assembled Monolayers on Gold-Coated Silicon Substrates

Silicon wafers (200 mm, P-type, Prime Grade Silicon 100) were diced to individual substrates and cleaned to afford a surface having fewer than 10 particles (0.16 μm to 3000 μm) per substrate. Metal deposition was carried out in a CPA 9900 sputtering system with a base pressure of 5×10⁻⁷ mm. In the sputtering chamber, the substrates were cleaned and etched by argon plasma and an adhesive layer of titanium and tungsten (1:9) was sputtered at a rate of 5 Å/s to a thickness of 250 Å. Gold was then sputtered at a rate of 20 Å/s up to a thickness of 1000 Å. Substrates were cooled under an argon flow prior to removal.

Prior to monolayer assembly, gold-coated substrates were cleaned by treatment with argon plasma at 200 W for 300 s. The substrates were rinsed with ethanol and then transferred to a 0.1 mM solution of 3 (11-(3,3,4,4,5,5,6,6,7,7,8,8,8-tridecafluoro-octyloxy)undecane-1-thiol) in ethanol and incubated at room temperature for a period ranging from 1 to 24 hours. Finally, surface-modified substrates were removed from the assembly bath, spin washed at 1000 rpm with ethanol and dried under a stream of nitrogen. The advancing contact angles of water drops (0.5 μL) applied to the surface-modified substrates were in the range 114° to 120°. Surface-modified substrates were stored in fitted plastic containers with transparent amber UV resistant covers.

### EXAMPLE XIV

### Preparation of Patterned Sample Presentation Devices

Twenty-four (24) surface-modified substrates were prepared as described above, mounted in a custom alignment jig and covered with a pin-registered etched stainless steel shadow mask (0.002 inch) having features corresponding in size and shape to the liquid retention zone. The jig was placed on the moving belt of an air-cooled ultraviolet curing system fitted with a low-pressure mercury light source rated at 120 W/cm² and passed under the light source 45 to 75 times over the course of one hour. Following UV exposure, the substrates were removed from the jig, spin washed at 1000 rpm with ethanol and dried under a stream of nitrogen. The exposed substrates were placed in a 0.1 mM solution of 6 (11-{2-[2-(2-methoxyethoxy)ethoxy]ethoxy}undecane-1-thiol) in ethanol and incubated at room temperature for a period ranging from 1 to 24 hours. Patterned surface-modified substrates were removed from the assembly bath, spin washed at 2400 rpm with ethanol and dried under a stream of nitrogen. The advancing contact angles of water drops applied to the liquid retention zone were in the range 60° to 65°, and when applied to the boundary zone were in the range 110° to 119°.

Patterned surface-modified substrates were mounted in a custom alignment jig and covered with a second pin-registered etched stainless steel shadow mask having features corresponding in size and shape to the analysis zone. The jig was placed on the moving belt of the ultraviolet curing system and passed under the light source 45 to 75 times over the course of one hour. Following UV exposure, the substrates were removed from the jig, spin washed at 1000 rpm with ethanol and dried under a stream of nitrogen. The exposed substrates were placed in a 0.1 mM solution of 9 (2-{2-[2-(11-mercaptoundecyloxy)ethoxy]ethoxy}ethanol) in ethanol and incubated at room temperature for 1-24 hours. Finally, twice-patterned surface-modified substrates were removed from the assembly bath, spin washed at 1000 rpm with ethanol and dried under a stream of nitrogen. The advancing contact angles of water drops applied to the analysis zone were less than 47°. Twice-patterned surface-modified substrates were stored in fitted plastic containers with amber transparent UV resistant covers.

While various embodiments of the present invention have been described above, it should be understood that they have been presented by way of example only, and not limitation. In particular, the physical arrangement of the analysis zone, liquid retention zone, and boundary zone is not limited by the examples described above. Thus, the breadth and scope of the present invention should not be limited by any of the above-described exemplary embodiments.

### EXAMPLE XV

### Sample Containment and Positioning

Analyte-confining properties of the analysis zone, which afford an increase in sensitivity of detection, are demonstrated in the video contact angle images shown in FIGS. 11a through 11h. With reference to FIG. 11a, the sample presentation device of the present invention was prepared with a liquid retention zone measuring about 1.6 mm OD and an analysis zone measuring about 0.7 mm OD. To facilitate the observation of the focusing effect, the analysis zone was placed off-center. A drop of water was applied to the surface of the biochip and was observed to rapidly confine itself to the surface area corresponding to the liquid retention zone and the analysis zone. The initial left-side and right-side contact angles were recorded and were both found to be 57.1°, a value which corresponds to that exhibited by a surface prepared from exclusively the liquid retention zone monomer. As the drop dried owing to evaporation (see FIGS. 11b through 11h), both the observed radius and contact angles receded until the radius of the drop corresponded to that of the analysis zone. Furthermore, as the drop dried it was observed that the center of the drop moved to the right so as to allow the drop to center itself over the analysis zone. The left-side and right-side contact angles recorded in FIG. 11h were both found to be 35.4°, a value which corresponds to that exhibited by a surface prepared exclusively from the analysis zone monomer. The drop height, width and contact angle data recorded in conjunction with the acquisition of the images depicted in FIGS. 11a through 11h is summarized graphically in FIG. 12.

### EXAMPLE XVI

### Liquid-Holding Capacity of Patterned Sample Presentation Devices

The extraordinary liquid-holding capacity of the liquid retention zone is demonstrated in FIG. 13. An illustration of a 16-site sample presentation device of the present invention shows the retention of sample drop volumes in the range 5 μL to 70 μL. The only factor that appears to significantly limit the sample drop volume is the relative proximity of the adjacent pairs of target and liquid retention zones.

### EXAMPLE XVII

### Analyte Directing and Concentration

Analyte-confining properties of the analysis zone are further demonstrated in FIGS. 14a and 14b. The first illustration (FIG. 14a) is of a 16-site sample presentation device of the present invention with sample drop volumes in the range 5 μL to 40 μL deposed on the surface of 8 of the 16 sites. Each of the liquid drops contained an equivalent amount of HCCA. FIG. 14b is an illustration of the HCCA having been concentrated and directed to the analysis zone due to sample drying on the sample presentation device depicted in FIG. 14a. The relative size of the analysis zone and the liquid retention zone is superimposed above the HCCA for comparison purposes.

### Capture Chips

In another aspect of the invention, the sample presentation device comprises one or more analyte binding regions. The binding regions may be applied to retain analytes for further processing and/or measurement on the sample presentation device. In one application, after the desired analytes are captured by a binding region, the surface of the sample presentation device is washed to remove undesirable species. The desired analytes are then released from the binding regions so they may be directed toward an analysis zone for further measurement and/or processing. Alternatively, the binding regions may be applied to filter/separate/remove undesirable species (e.g., salts, detergents, proteins, etc.). In one application, at least portion of the undesired species are retained in a capture zone while the rest of the sample, which carries the desired analytes, is allowed to move toward the analysis zone for measurements and/or processing.

In one variation, of the sample presentation devices can be termed “capture chips” or “capture/concentrate chips,” and abbreviated Xn where “n” is a numerical designation referring to the number of zones on the surface of the sample presentation device, where “n” can be any number from 2 to infinity. Thus, for example, an X2 target chip has two zones, an X3 target chip has three zones, etc. The present invention contemplates sample presentation devices containing many more than 2 or 3 zones and is not limited in any way to a specific number of zones. As the number of zones increases, the overall effect approaches a gradient. Capture chips and capture/concentrate chips are sample presentation devices comprised of one or more zones that are designed to bind analytes. The moieties responsible for capturing analytes typically comprise specific surface modifications that are designed as the distinguishing feature of the capture zone. These surface modifications may comprise biological and/or chemical moieties that bind analytes specifically (such as monoclonal antibodies) or non-specifically (such as charged groups that bind on the basis of electrostatic attraction) or any combination of such attractive forces. These bound analytes may then be further processed on the surface prior to analysis. These processing steps include, but are not limited to, purification of the analyte of interest through various washing steps, modification of the analyte by chemical, biochemical, or physical methods, and isolation for subsequent use. The bound analytes may then be released after being subjected to chemical or physical stimuli such as changes in pH, changes in solvent composition, UV radiation, electricity, or heat. Upon release, the analytes may then be concentrated to the analysis zone upon solvent evaporation.

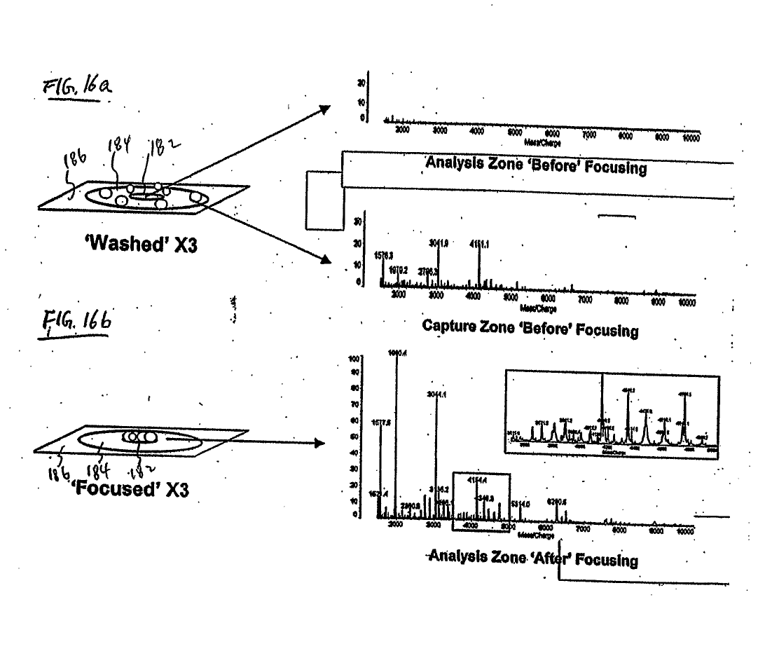

In one variation illustrated in FIG. 15, the capture chip 180 comprises a surface having an analysis zone 182, a capture zone 184 forming a concentric circle around the analysis zone 182, and a boundary zone 186 surrounding the capture zone 184, as shown in step (a) of FIG. 15. The SAM in the capture zone 184 comprises binding areas 188 for capturing analytes (e.g., chemicals, biochemical, etc.). FIG. 15 illustrates an example of using the capture zone 184 to extract desired molecules from a liquid and then concentrate the desired molecules onto the analysis zone 182. A fluid 190 containing the analytes of interest 192 (e.g., proteins, peptides, etc.) and undesired molecules 194, 196, 198 (e.g., salts, detergents, contaminants, etc.) is placed within the boundary defined by the capture zone 184, as shown in step (b) of FIG. 15. The analytes 192 bind to the functional groups on the surface of the capture zone 184, as shown in step (c) of FIG. 15. After a period of time is provided to allow this binding interaction to occur, the liquid

200 carrying the undesired molecules 194, 196, 198 is then washed away, as shown between step (c) and (d) of FIG. 15. A new drop of liquid 202, free of undesired molecules, is then introduced onto the capture zone 184 and the analysis zone 182, as shown in step (d) of FIG. 15. The analytes of interest 192 are then released from the surface of the capture zone 184. This release of the desired molecules 192 may be achieve through introduction of chemicals in the new drop of liquid 202, introduction of liquid or solvent with particular intrinsic properties which facilitates the release of desired molecules, photon excitation, change in pH, change in temperature, or other chemical and/or physical changes. The liquid is then removed from the capture zone 184 and the analysis zone 182 through evaporation or other means that are well known to on of ordinary skill in the art, leaving the desired molecules 192 to concentrate onto the analysis zone 182, as shown in step (e) of FIG. 15.

Furthermore, SAMs may be preferred for protein assays. Self-assembling molecular layers may be formed directly on a substrate. Preferably, the substrate is a metal, such as gold, or a semiconductor, such as silicon, but may include a variety of materials, including but not limited to, for example, glasses, silicates, semiconductors, , metals, polymers (e.g., plastics), and other hydroxylated materials, e.g., SiO₂ on silicon, Al₂O₃ on aluminum, etc. However, it is preferred to form self-assembling monolayers of alkylthiols on gold, or organosilanes on silicon or glass. The SAMs may be added to the sample presentation devices of the present invention in a manner that creates distinct zones whose properties reflect the SAMs used in a particular zone. Molecules used to form self-assembling layers may have a reactive group such as a terminal thiol or silane group that reacts with the substrates surface. A hydrocarbon chain, such as an alkyl moiety, may also form part of a molecule. The molecules also may have oligomeric or polymeric chains with little or no side-branching. These chains may optionally also have a functional group that may be directly employed as a capture moiety (e.g. biotin or antibodies) or further derivatized to create a capture surface. A sample presentation device of the present invention that comprises a capture zone in which the surface modification is a monoclonal antibody may bind a complimentary antigen from a liquid sample and retain that antigen while the rest of the liquid sample moves to another part of the surface of the device, through either physical transfer or differences in wettability. The retained antigen may subsequently be modified via the addition of other compounds to the capture zone of the sample presentation device (e.g., the addition of an enzyme that cleaves off a part of the antigen). The modified antigen can then be transferred into the analysis zone, or be used as a capture surface itself. Similarly, a sample presentation device of the present invention that comprises a capture zone in which the surface modification is biotin may bind a streptavidin-linked analyte from, a liquid sample and retain that analyte while the rest of the liquid sample moves to another part of the surface of the device, through either physical transfer or differences in wettability. The retained streptavidin-linked analyte may subsequently be modified via the addition of other compounds to the capture zone of the sample presentation device. The modified analyte can then be transferred into the analysis zone, or be used as a capture surface itself.

A sample presentation device of the present invention that comprises a capture zone which is created through a convergent technique may be created through surfaces presenting reactive groups on their termini. These could include, but are not limited to, amine-terminated and carboxy-terminated SAMs. Amine-reactive or carboxy-reactive species may be introduced to subsequently create the capture zones surface. Thus, an amine-terminated surface may be treated with a copolymer containing maleic anhydride moieties and various other group including, but not limited to, short-chain (C₄-C₁₁)alkyl groups which may be partially saturated, long-chain (C₁₂-C₂₄)alkyl groups which may be partially saturated, aromatic groups which may or may not be substituted, charged groups, nucleophilic groups, electrophilic groups, etc. This would lead to the copolymer being bound to the surface via the amide bond formed with the amine-terminus and presenting the functional portion of the polymer for use as a capture surface.

In one particular application, the SAM in the boundary zone 186 comprises 11-(3,3,4,4,5,5,6,6,7,7,8,8,8-Tridecafluorooctyloxy)undecane-1-thiol (Example III), the SAM in the capture zone 184 comprises of carboxy terminated polyether alkyl thiolate (Whitesides G. M., Lahiri J., Isaacs L., Tien J. Anal. Chem. 1999, 71 (4), 777-790), and the SAM in the analysis zone 182 comprises of hydroxy terminated polyether alkyl thiolate (Example IX). A sample liquid droplet containing undesired molecules, such as sodium chloride and sodium dodecylsulfate, along with the analytes of interest, e.g. peptides and proteins from serum, may be provided for processing on this capture chip. The process shown in FIG. 15 may then be performed to extract the desired molecule for analysis with mass-spectrometry. After the undesired molecules are removed through the “Wash Sample” step, mass spectrometry may be performed on the capture zone 184 and which would lead to the spectrum shown in FIG. 16a. The “Focus Analyte” step is performed to concentrate the analytes onto the analysis zone 182. Mass spectrometry may then be performed on the analysis zone 182 which would yield a spectrum similar to the one shown in FIG. 16b. Comparing the spectrums in FIG. 16b to FIG. 16a, one can see that the focusing step may significantly improved signal to noise ratio of the mass spectrometry measurements of the desired molecules. Although the above example of the capture chip configuration may be particularly useful for use with serum samples, one of ordinary skill in the art would appreciate that this capture chip may also be implemented in other application where purification and/or concentration of the analyte is desirable before performing an analysis or measurement on the analyte.

In yet another variation, the analysis zone is configured with a binding surface for capturing and/or binding to analytes, while the liquid retention zone and other surface regions surrounding the analysis zone are covered with SAM having substantially nonbinding characteristics. This design may allow the sample liquid droplet to concentrate onto the analysis zone and further allow the analytes to bind to the surface of the analysis zone. Mass-spectrometry or other detection mechanisms may then be performed on the analytes captured on the surface of the analysis zone.

In another variation, both the analysis zone and the liquid retention zone are configured with binding/capturing surfaces. For example, the liquid retention zone may be configured to bind to one type of molecule while the analysis zone may be configured to bind to a different type of molecule. These configurations with multiple binding zones may be applicable in applications where the analysis zone is implemented to capture the desired molecules, while the liquid retention zone is implemented to capture one or more undesirable molecules. Alternatively, one region is used to capture one type of molecules, while the other region is used to capture a different type of molecules for analysis. The molecules may be analyzed right on the binding area. Optionally, the different types of captured molecules may be selectively released from the binding area and transported to an analysis zone for further processing or measurement.

Another aspect of the invention comprises methods for filtering and/or focusing analytes (e.g., chemicals, biochemical, biologics, etc.) on a surface. Preferably, the method is performed on a surface having a plurality of SAM regions. In one variation, the method comprises the following steps: First, deliver a liquid comprises desired analytes and undesired species onto a surface; then capture or bind the desired analytes on a first region of the surface; the undesired species are the washed off; the desired analytes are released from the first region and directed toward a second region on the surface; the desired analytes may be allowed to focus and/or concentrate onto the second region; analysis and/or further processes may then be conducted on the desired analytes. The liquid may be directed to flow on the surfaces due to variation in liquid surface tension in different areas on the surface. Preferably, the second region is substantially non-binding. In addition, the method may be performed on a surface comprises a series concentric rings defined by different SAMs.

In another variation, a liquid comprises both desired analytes and undesired species are introduced onto a surface. At least portion of the undesired species are captured on a first region on the surface. The liquid carrying the desired analytes is directed toward a second region through variation in liquid surface tension in the different areas on the surface. The desired analytes may then be concentrated and/or focused onto the second surface region. Analysis and/or further processes may then be conducted on the desired analytes in the second region. The liquid may be directed to flow on the surfaces due to variation in liquid surface tension in different areas on the surface. Preferably, the second region is substantially non-biding. In addition, the method may be performed on a surface comprises a series of concentric rings defined by different SAMs. Various methods for utilizing the sample presentation device are also described herein.

### EXAMPLE XVIII

### Preparation of one Variation of a Capture Chip

Twenty-four (24) surface-modified substrates were prepared as described in example XIII, mounted in an alignment jig and covered with a pin-registered etched stainless steel shadow mask (0.002 inch) having features corresponding in size and shape to the liquid retention zone. The jig was placed on the moving belt of an air-cooled ultraviolet curing system fitted with a low-pressure mercury light source rated at 120 W/cm² and passed under the light source 45 to 75 times over the course of one hour. Following UV exposure, the substrates were removed from the jig, spin washed at 1000 rpm with ethanol and dried under a stream of nitrogen. The exposed substrates were placed in a 0.1 mM solution of 11-amino-1-undecanethiol (Dojindo Molecular Technologies, Inc. product code A423-10) in ethanol and incubated at room temperature for a period ranging from 1 to 24 hours. Patterned surface-modified substrates were removed from the assembly bath, spin washed at 2400 rpm with ethanol and dried under a stream of nitrogen. The advancing contact angles of water drops applied to the liquid retention zone were in the range 48° to 55°, and when applied to the boundary zone were in the range 110° to 119°.

The patterned surface-modified substrates from above were mounted in an alignment jig and covered with a second pin-registered etched stainless steel shadow mask having features corresponding in size and shape to the analysis zone. The jig was placed on the moving belt of the ultraviolet curing system and passed under the light source 45 to 75 times over the course of one hour. Following UV exposure, the substrates were removed from the jig, spin washed at 1000 rpm with ethanol and dried under a stream of nitrogen. The exposed substrates were placed in a 0.1 mM solution of 6 (11-{2-[2-(2-methoxyethoxy)ethoxy]ethoxy}undecane-1-thiol) in ethanol and incubated at room temperature for 1-24 hours. Finally, twice-patterned surface-modified substrates were removed from the assembly bath, spin washed at 1000 rpm with ethanol and dried under a stream of nitrogen. The advancing contact angles of water drops applied to the analysis zone were less than 47°.

These twice-patterned surfaces were then exposed to a solution of a long-chain poly(alkyl) co-polymer containing amine-reactive sites (1 mg/mL in dichloromethane containing 0.5% v/v pyridine) at room temperature for four hours. The substrates were subsequently washed with warm chloroform (2×), followed by a wash in a mixture of acetonitrile/ethanol/water (84:13:3), and then they were placed in a 10% solution of ammonia for 30 minutes. The ammonia solution was replaced by three subsequent water washes and the substrates were subjected to ethanol spinwashing (vide supra). The advancing contact angles of water drops applied to the liquid retention zone were 84-89°.

### EXAMPLE XIX

### Purification of Analyte on a Capture Chip

In MALDI-TOF spectroscopy, the ionization, and thus detection, of analytes (e.g.; peptides, proteins, sugars, oligonucleotides, etc.) may be substantially influenced by the purity and composition of the sample to be analyzed. Materials such as salts (e.g.; sodium chloride), detergents (e.g.; sodium dodecylsulfate, SDS), buffers (e.g. TRIS) and urea may fully suppress the detection of analytes, especially in more dilute samples. These materials may be endogenous to the sample or introduced during some sample processing steps and necessitates an additional purification step prior to MALDI-TOF analysis. Thus, current technology utilized for this process routinely includes reversed-phase liquid chromatography or solid phase extraction, including ZipTips, to remove these undesirable contaminants. When employing these additional sample purification steps, a T3 surface, whose volume holding capabilities are within the effluent volumes common to these processing steps, may be used to perform the analysis. However, the use of an X3 surface containing long-chain allyl (LCA) groups in the liquid retention zone (LRZ) may facilitate the detection of analytes from samples containing these contaminants. By allowing for the on-surface purification and subsequent focusing of sample for MALDI-TOF analysis, the need for the extra processing steps (and equipment and materials) may be obviated.

An example illustrating the use of a capture chip to analyze peptides is illustrated below. Yeast enolase digest (Waters Corp. part no. 186002325, SwissProt P00924) was dissolved in mixed solutions of 20% acetonitrile (ACN)/80% aqueous containing different contaminants. The aqueous portions all contained 0.1% trifluoroacetic acid (TFA) as well as one of the following: 1 M sodium chloride (NaCl), 1 M urea (Urea), 1 M TRIS buffer (TRIS), and 0.1% sodium dodecylsulfate (SDS). Thus, enolase digest was dissolved in each of these four solutions and the final concentration of analyte was adjusted to 1 fmol/μL.

The analyte-containing solutions were applied to the liquid retention zone (LRZ) of the X3 chip described in example XVIII (16 sites in a 2×8 array) in 10 μL aliquots (10 fmol total analyte) and allowed to incubate on the surface for 20 minutes. In parallel, the same solutions were applied to a stainless steel MALDI platform (Bruker Daltonics part no. 26755) in order to compare the effects. Due to the volume limitations of the stainless steel surface, the full 10 μL of sample could not be applied.

Following incubation on the X3 chip, the majority of the solution was removed and then the sites were washed with 0.1% TFA (3×) via pipet. Residual liquid on the surface after the third wash was allowed to dry and then 2 μL of a solution containing the matrix α-cyano-4-hydroxycinammic acid (CHCA, Waters Corp. part no. 186002331, 0.25 mg/mL in acetonitrile/ethanol/0.1% TFA 84:13:3) was applied to each site. The matrix solution spread to fill the 3.0 mm diameter of the site. Upon drying, the sample and matrix were concentrated into the analysis zone (AZ) of the X3 site with which it was associated. The sample was then secured in a holder in preparation for MALDI-TOF analysis. Similarly, washes were attempted on the MALDI stainless steel plate, matrix was applied and the plate was prepared for MALDI-TOF analysis.

MALDI-TOF analysis was performed in the positive ion mode on an Axima CFR (Kratos Analytical by Shimadzu Biotech, Manchester, UK) using a pulsed N₂ laser (337 μnm), delayed extraction, and an acceleration voltage of 20 kV. The instrument was operated in reflectron mode using a semi-automated protocol producing, generally, 25-50 raster points/site, 20-50 shots per raster point. Data was collected and stored as an average of all raster points and then subjected to analysis by peptide mass fingerprinting (PMF) using MASCOT (http://www.matrixscience.com). MASCOT is a search engine which uses observed fragments (peaks) in the mass spectrum in order to identify proteins from their primary sequences.

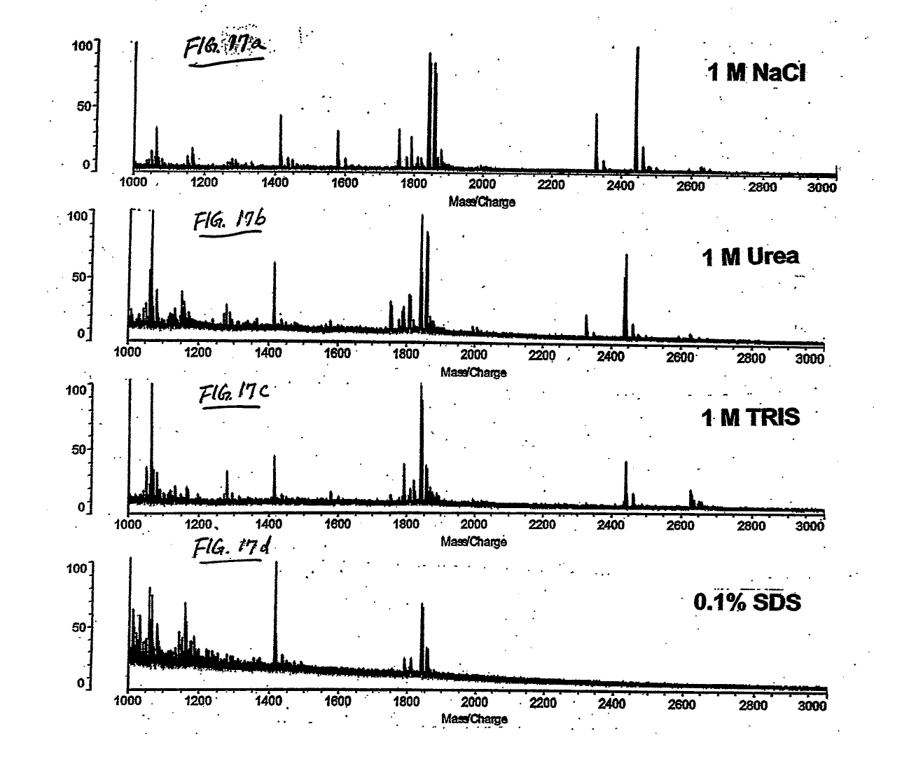

In all four cases, direct analysis on stainless steel resulted in useless data. Each contaminant caused a noisy spectrum in which real peaks were not discernable from the noise in the spectrum. Direct application of the clean (i.e., not intentionally contaminated) digest to a stainless steel surface did result in spectra in which PMF could be performed. In contrast, the X3 surface provided spectra which were high in signal to noise (S/N) ratio and rich in peaks. FIG. 17a -17d illustrates the spectra produced after on-surface purification in the presence of the indicated contaminant. FIG. 17a shows the result obtained from the sample contaminated with 1M NaCl, FIG. 17b shows the result obtained from the sample contaminated with 1M Urea, FIG. 17c shows the results obtained from the sample contaminated with 1M TRIS, while FIG. 17d shows the results obtained from the sample contaminated with 0.1% SDS. MASCOT analysis of these spectra from the X3 surface all identified the correct digested protein as the highest probability answer. These results illustrate the capability of the X3 surface to directly analyze peptides of interest in the presence of common contaminants without the need for an additional purification steps.

### EXAMPLE XX

### Direct Application and Analysis of an In-Gel Digested Protein

Enzymatic digestion of proteins in gels is a common approach utilized in the field of proteomics. Upon completion of 1-D or 2-D electrophoresis and staining, gel pieces are excised and destained prior to digestion, and then subsequently reduction and alkylation of the free thiols. The resultant peptides may then be solubilized and released into acidic solutions. Because the samples, at this stage, are often dilute and contain contaminants (e.g. salts, small molecules, etc.), a purification and concentration scheme may be beneficial prior to analysis by MALDI-TOF. Current methods for this purification include liquid phase chromatography or the use of solid-phase extraction cartridges. Liquid chromatography is an instrument-dependent process and the commercially available solid-phase extraction products (e.g. ZipTip) can suffer from tip-to-tip performance. This requirement may render the analysis to a low-throughput process. Thus, a sample measurement surface with the capability to remove at least part of these interfering contaminants may be desirable. Such a sample measurement surface may provide a reproducible and high-throughput platform for analyte detection and measurement.

In one particular application, Phosphorylase b from Rabbit muscle (Sigma part no. P6635, SwissProt P00489) was dissolved into 18 MΩ water to give a stock solution. This stock was then used to prepare the protein for 1-D gel electrophoresis. Thus, Laemmli buffer (Bio-Rad part no. 161-0737) was prepared according to the manufacturer's protocol and used to dilute the protein stock prior to use. Gel electrophoresis was performed using a pre-cast 4-15% SDS PAGE gel in Tris•HCl buffer (Bio-Rad product no. 161-1176) at 70 V constant voltage. Upon completion of the run, the gel was stained using Gel Code Blue (Pierce product no. 24590) overnight and subsequently washed with water. The material was then prepared for an in-gel digestion using trypsin. A commercially available in-gel tryptic digestion kit (Pierce product no. 89871X) was employed and the manufacturer's protocol was followed, but the sample was not subjected to liquid chromatographic purification. The final concentration of the digested sample stock was approximately 7.5 pmol/μL and dilutions were performed to give a working solution concentration of 7.5 fmol/μL in 25% acetonitrile/0.1% TFA. A portion of the digested sample stock was also passed through a ZipTip_{μC18} (Millipore part no. ZTC 18M) following the manufacturer's protocol. The concentration of this sample was then adjusted to 7.5 fmol/μL in 25% acetonitrile/0.1 % TFA. This cleaned sample would then be used on a T3 surface for comparison purposes.

The analyte-containing solution (10 μL) was applied to the liquid retention zone (LRZ) of the X3-type described in example XVIII (75 mmol total analyte) and allowed to incubate on the surface for 20 minutes. Following incubation, the majority of the solution was removed and then the sites were washed with 10 μL of 0.1% TFA (3×) via pipet. Residual liquid on the surface after the third wash was allowed to dry and then 2 μL of a solution containing the matrix α-cyano-4-hydroxycinammic acid (CHCA, Waters Corp. part no. 186002331, 0.065 mg/mL in acetonitrile/ethanol/0.1% TFA 84:13:3) was applied to each site. The matrix solution spread to fill the 3.0 mm diameter of the site. Upon drying, the sample and matrix were concentrated into the analysis zone (AZ) of the X3 site with which it was associated. The sample surface was then secured in a holder in preparation for MALDI-TOF analysis.

Similarly, the cleaned digest sample (10 μL) was applied to the liquid retention zone (LRZ) of the T3 (75 fmol total analyte) and allowed to focus and dry. Then, 2 μL of a solution containing the matrix α-cyano-4-hydroxycinammic acid (CHCA, Waters Corp. part no. 186002331, 0.065 mg/mL in acetonitrile/ethanol/0.1% TFA 84:13:3 containing 10 mM ammonium citrate) was applied to each site. The matrix solution spread to fill the 3.0 mm diameter of the site. Upon drying, the sample and matrix were concentrated into the analysis zone (AZ) of the T3 site with which it was associated. The sample surface was then secured in a holder in preparation for MALDI-TOF analysis.

MALDI-TOF analysis was performed in the positive ion mode on an Axima CFR (Kratos Analytical by Shimadzu Biotech, Manchester, UK) using a pulsed N₂ laser (337 nm), delayed extraction, and an acceleration voltage of 20 μV. The instrument was operated in reflectron mode using a semi-automated protocol producing, generally, 25-50 raster points/site, 20-50 shots per raster point. Data was collected and stored as an average of all raster points and then subjected to analysis by peptide mass fingerprinting (PMF) using MASCOT (http://www.matrixscience.com). MASCOT is a search engine which uses observed fragments (peaks) in the mass spectrum in order to identify proteins from their primary sequences.

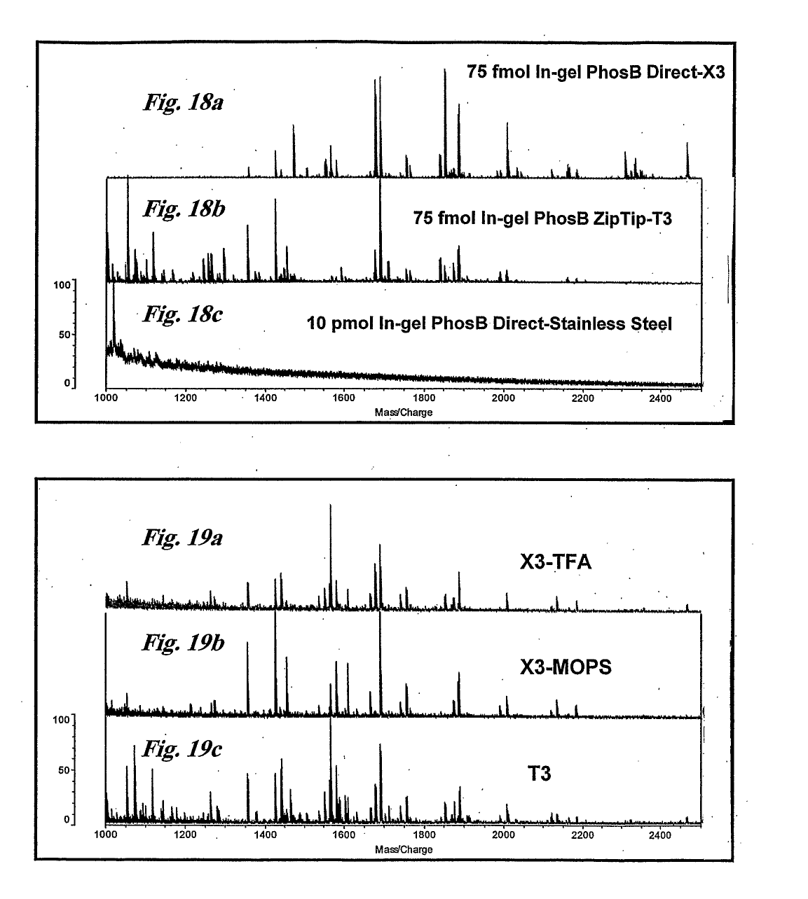

Direct application of sample to the stainless steel surface did not produce discernable spectra. An example of a 10 pmol sample is shown in FIG. 18c. All concentrations less than this on stainless steel resulted in only noise in the spectrum. The X3 surface was capable of purifying peptides and focusing them into the analysis zone (AZ). As shown in FIG. 18a, the spectra is rich in fragment peaks, especially above m/z values of 1300 Daltons. The ZipTip/T3 method also resulted in a peak-rich spectrum, but the mass range is skewed towards the lower molecular weight (less than 2000 Daltons) species, as shown in FIG. 18b.

Although the spectra resulting from an X3 protocol or ZipTip/T3 protocol do vary in appearance, both methods are suitable for this type of analysis. The use of a ZipTip for desalting in combination with a T3 surface gave results in which a 37% of the observed peaks match the theoretically determined fragments. The matched peptides correspond to a 37% sequence coverage of the expected tryptic digest fragments. Direct application of the in-gel digest to an X3 surface produced spectra where 44% of the peaks matched the theoretically predicted fragments accounting for a 39% sequence coverage. These values are comparable to those obtained by the process of purifying on a ZipTip and transferring to a MALDI sample plate, but without the extra sample preparation step.

### EXAMPLE XXI

### Expanded Mass Range Coverage of an X3 surface

### X3-Acetonitrile-TFA Protocol

Rabbit phosphorylase B (Waters Corp. part no. 186002326, SwissProt P00489) was dissolved in a mixed solution of 50% acetonitrile (ACN)/50% trifluoroacetic acid and the final concentration adjusted to 1 fmol/μL. The analyte-containing solution (10 μL) was applied to the liquid retention zone (LRZ) of the X3-type surface described in example XVIII (10 fmol total analyte) and allowed to incubate on the surface for 20 minutes. Following incubation, the majority of the solution was removed and then the sites were washed with 10 μL of 0.1% TFA (2×) via pipet. Residual liquid on the surface after the third wash was allowed to dry and then 2 μL of a solution containing the matrix 2,5-dihydroxybenzoic acid (DHB, Waters Corp. part no. 186002333, 0.65 mg/mL in acetonitrile/0.1% TFA containing 10 mM ammonium citrate, 4:1) was applied to each site. The matrix solution spread to fill the 3.0 mm diameter of the site. Upon drying, the sample and matrix were concentrated into the analysis zone (AZ) of the X3 site with which it was associated. The sample surface was then secured in a holder in preparation for MALDI-TOF analysis.

### X3-Acetonitrile-MOPS Protocol

Alternately, rabbit phosphorylase B (Waters Corp. part no. 186002326, SwissProt P00489) was dissolved in a mixed solution of 50% acetonitrile (ACN)/50% 3-(N-morpholino)propanesulfonic acid (MOPS, 200 mM, pH 6.5) and the final concentration adjusted to 1 fmol/μL. The analyte-containing solution (10 μL) was applied to the liquid retention zone (LRZ) of the X3-type surface described in example XVIII (10 fmol total analyte) and allowed to incubate on the surface for 20 minutes. Following incubation, the majority of the solution was removed and then the sites were washed with 10 μL of 20 mM ammonium acetate (2×) via pipet. Residual liquid on the surface after the third wash was allowed to dry and then 2 μL of a solution containing the matrix 2,5-dihydroxybenzoic acid (DHB, Waters Corp. part no. 186002333, 0.65 mg/mL in acetonitrile/0.1% TFA containing 10 mM ammonium citrate, 4:1) was applied to each site. The matrix solution spread to fill the 3.0 mm diameter of the site. Upon drying, the sample and matrix were concentrated into the analysis zone (AZ) of the X3 site with which it was associated. The sample surface was then secured in a holder in preparation for MALDI-TOF analysis.

### T3-Acetonitrile-TFA Protocol

Rabbit phosphorylase B (Waters Corp. part no. 186002326, SwissProt P00489) was dissolved in a mixed solution of 50% acetonitrile (ACN)/50% trifluoroacetic acid and the final concentration adjusted to 1 fmol/μL. The analyte-containing solution (10 μL) was applied to the liquid retention zone (LRZ) of the T3-type surface described in example XIII (10 fmol total analyte) and allowed to dry and focus into the analysis zone. Then, 2 μL of a solution containing the matrix 2,5-dihydroxybenzoic acid (DHB, Waters Corp. part no. 186002333, 0.65 mg/mL in acetonitrile/0.1% TFA containing 10 mM ammonium citrate, 4:1) was applied to each site. Upon drying, the sample and matrix were concentrated into the analysis zone (AZ) of the T3 site with which it was associated. The sample surface was then secured in a holder in preparation for MALDI-TOF analysis.

MALDI-TOF analysis was performed one the above samples in the positive ion mode on an Axima CFR (Kratos Analytical by Shimadzu Biotech, Manchester, UK) using a pulsed N₂ laser (337 nm), delayed extraction, and an acceleration voltage of 20 kV. The instrument was operated in reflectron mode using a semi-automated protocol producing, generally, 25-50 raster points/site, 20-50 shots per raster point. Data was collected and stored as an average of all raster points and then subjected to analysis by peptide mass fingerprinting (PMF) using MASCOT.

Both methods on the X3-type surface gave spectra rich in peaks with a high signal to noise ratio, but without a complete overlapping of sequences. The TFA protocol gives a spectrum with a 44% sequence coverage, as shown in FIG. 19a. The MOPS protocol resulted in a spectrum with 46% sequence coverage, but, more importantly, revealed new information about the sample itself, as shown in FIG. 19b. When one combines the identified fragments from each protocol, the sequence coverage increases to 57% of the expected fragments from a tryptic digestion of phosphorylase b. This is in good agreement with the results obtained from a T3-type surface after applying a clean digest sample, as shown in FIG. 19c. In this instance, the sequence coverage is 58% and may be representative of the readily detectable fragments of interest from the sample.

### Examples Utilizing Antibodies as Capturing Mechanisms

Specific isolation and detection of analytes from complex solutions (e.g., serum, mixed proteolytic digests, plasma, etc.) is crucial to various biological, biochemical and chemical analysis processes. Antibodies are among the most selective and sensitive tools available, and can be readily be generated against a variety of analytes. Antibodies form the basis of immunoassays, which are widely employed in clinical chemistry, environmental analysis, and research and development. Analytes purified with antibodies typically require one or more off-line clean-up and concentration steps before application to an analysis substrate (e.g. MALDI substrate, etc.), which may result in significant sample loss.

A device capable of capturing analytes and performing the concentration steps on a single substrate may minimize sample loss and improve detection capability. Antibodies and other ligands which specifically capture analytes can be immobilized onto a surface on a sample presentation device to form the capture zone. For example, one may modify an X3 surface through a variety of chemistries to create a capture zone with immobilized antibodies. The X3 chip may then be utilized to provide selective capture, concentration, and presentation for MALDI-MS on a single device, which may eliminate the need for additional processing steps.

In one example, the following series of chemicals were synthesized to generate chemicals for the preparation of a sample presentation device with a surface for anchoring antibodies.

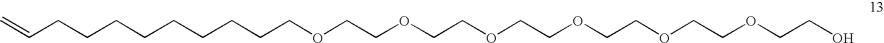

### 2-[2-(2-{2-[2-(2-Undec-10-enyloxyethoxy)ethoxy]ethoxy}ethoxy)ethoxy]ethanol (13).

A 100 mL round bottom flask was charged with hexaethyleneglycol (26.9 mL, 0.1 mol) and to this was added 50% aqueous sodium hydroxide (1.72 mL, 22 mmol). The solution was warmed to 100° C. and stirred for 30 minutes. At this time, 11-bromoundec-1-ene (4.7 mL, 21 mmol) was added dropwise and the reaction continued at 100° C. for 18 hours until TLC analysis showed the starting material to be consumed, and then cooled to room temperature. The solvent was evaporated in vacuo to give an oily residue. This residue was subjected to column chromatography (SiO₂, 40×200 mm, 20% methanol/ethyl acetate), the fractions containing the desired product were then combined, and the solvent was evaporated to give 2.0 g (21%) of 13 as a light yellow oil. ¹H NMR (400 MHz, CDCl₃) δ 5.78 (m, 1H), 4.94 (m, 2H), 3.69-3.54 (m, 24H), 3.42 (t, J=7.0 Hz, 2H), 2.38 (bs, 1H), 2.01 (m, 2H), 1.55 (m, 2H), 1.33 (m, 2H), 1.23 (bs, 10H).

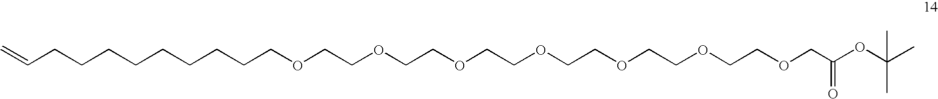

### {2-[2-(2-{2-[2-(2-Undec-10-enyloxyethoxy)ethoxyl ethoxy}ethoxy)ethoxy]ethoxy}-acetic acid tert-butyl ester (14)

A 50 mL round bottom flask was charged with 13 (2.0 g, 4.6 mmol) under anhydrous conditions. This was dissolved into 10 mL of dry dimethylformamide and cooled to 0° C externally. To this cold solution was added sodium hydride (60% in mineral oil, 267 mg, 6.9 mmol) in one portion and the frothing mixture was allowed to stir under argon at 0° C. for 10 minutes. At this time, tert-butylbromoacetate (1.02 mL, 6.9 mmol) was added dropwise and the reaction was warmed to 20° C. for 8 hours. TLC analysis showed that the reaction had quit progressing at this point. The reaction was quenched by the slow addition of 10 mL of water, diluted with 50 mL of ethyl acetate, and extracted with water (2×50 mL). The organic layer was dried over magnesium sulfate, filtered, and the solvent evaporated to give an oily residue. The residue was then subjected to column chromatography (SiO₂, 40×200 mm, 100% ethyl acetate), the fractions containing the desired product were then combined, and the solvent was evaporated in vacuo to give 1.64 g (65%) of 14 as a colorless oil. ¹H NMR (400 MHz, CDCl₃) δ 5.79 (m, 1H), 4.94 (m, 2H), 4.00 (s, 2H), 3.71-3.54 (m, 24H), 3.42 (t, J=6.8 Hz, 2H), 2.01 (m, 2H), 1.54 (m, 2H), 1.45 (s, 9H), 1.34 (m, 2H), 1.24 (bs, 10H).

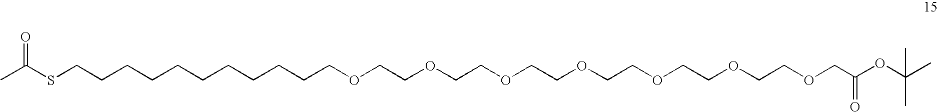

### (2-{2-[2-(2-{2-[2-(11-Acetylsulfanylundecyloxy)ethoxy]ethoxy}ethoxy)ethoxy]-ethoxy}ethoxy)acetic acid tert-butyl ester (15)

A 50 mL round bottom flask was charged with 14 (1.64 g, 3.0 mmol) under anhydrous conditions and this was dissolved into 20 mL of dry methanol. To this was added 2,2′-azobis(2-methylpropionamidine)dihydrochloride (81 mg, 0.3 mmol) followed by thiolacetic acid (715 μL, 10 mmol). The reaction was then shrouded in a foil tent and treated with light from a low pressure mercury lamp. After 4 hours, TLC analysis showed that the starting material had been consumed. The solvent was evaporated in vacuo to give an oily residue. The residue was then subjected to column chromatography (SiO₂, 40×200 mm, 100% ethyl acetate), the fractions containing the desired product were then combined, and the solvent was evaporated to give 1.48 g (79%) of 15 as a colorless oil. ¹H NMR (400 MHz, CDCl₃) δ 4.02 (s, 2H), 3.73-3.56 (m, 24H), 3.44 (t, J=6.8 Hz, 2H), 2.86 (t, J=7.2 Hz, 2H), 2.32 (s, 3H), 1.56 (m, 4H), 1.48 (s, 9H), 1.25 (bs, 14H).

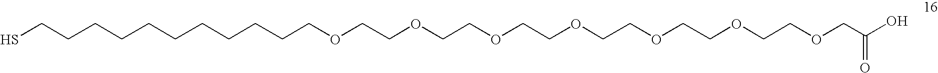

### (2-{2-[2-(2-{2-[2-(11-Mercaptoundecyloxy)ethoxy]ethoxy}ethoxy)ethoxy]-ethoxy}ethoxy)acetic acid (16).

A 20 mL amber shell vial with a teflon/silicon septa cap was charged with 15 (2.0 g, 3.2 mmol), and this was dissolved into 3 N methanolic hydrogen chloride (5 mL, 15 mmol) and warmed to 50° C. The solution was kept at 50° C. for 2 hours. The solvent was then removed in vacuo and the residue was then dissolved into 5 mL of a 50% aqueous potassium hydroxide in methyl sulfoxide (1:1) which had been deoxygenated prior to addition of the residue. The mixture was stirred at room temperature for 1 hour and then the solvent was evaporated in vacuo to give 1.6 g (95%) of 16 as a clear oil. The compound did not require further chromatographic purification. ¹H NMR (400 MHz, CDCl₃) δ 9.98 (bs, 1H), 4.14 (s, 2H), 3.74-3.57 (m, 24H), 3.44 (t, J=6.8 Hz, 2H), 2.51 (q, J=7.2, 2H), 1.57 (m, 4H), 1.32 (t, J=7.6 Hz, 1H), 1.26 (bs, 14H).

### EXAMPLE XXII

### Preparation of Patterned Sample Presentation Devices

### (X3 style, NHS (N-hydroxysuccinimide) Example)

In the process of fabricating sample presentation devices with antibody based capture zone, one may first prepare devices with its intended capture zones modified for receiving and anchoring antibodies. The prefabricated sample presentation devices may then be customized with specific antibodies depending on the intended application. The prefabricated sample presentation device may be utilized in a mass-production process to minimize manufacturing costs. For example, a large number of the prefabricated sample presentation devices may be produced first, and then selective batches of the production may then be customized with specific types of antibodies. In another application, the prefabricated sample presentation device may be provided to the end user. The end user may then customize the prefabricated sample presentation device by anchoring specific antibodies in the capture zone of the sample presentation device.

An example of a method for creating a prefabricated sample presentation device is described below. In this example, the capture zones on the sample presentation device are derivatized with NHS-ester groups such that they may be utilized to anchor antibodies. Twenty-four surface-modified substrates were prepared as described in example XIII, mounted in a custom alignment jig and covered with a pin-registered etched stainless steel shadow mask (0.002 inch) having features corresponding in size and shape to the liquid retention zone. The jig was placed on the moving belt of an air-cooled ultraviolet curing system fitted with a low-pressure mercury light source rated at 120 W/cm² and passed under the light source 45 to 75 times over the course of one hour. Following UV exposure, the substrates were removed from the jig, spin washed at 1000 rpm with ethanol and dried under a stream of nitrogen. The exposed substrates were placed in a mixed solution containing 5% of 16 and 95% of 6 (0.1 mM total thiol concentration) in ethanol and incubated at room temperature for a period ranging from 1 to 24 hours. Patterned surface-modified substrates were removed from the assembly bath, spin washed at 2400 rpm with ethanol and dried under a stream of nitrogen.

Patterned surface-modified substrates were mounted in a custom alignment jig and covered with a second pin-registered etched stainless steel shadow mask having features corresponding in size and shape to the analysis zone. The jig was placed on the moving belt of the ultraviolet curing system and passed under the light source 45 to 75 times over the course of one hour. Following UV exposure, the substrates were removed from the jig, spin washed at 1000 rpm with ethanol and dried under a stream of nitrogen. The exposed substrates were placed in a 0.1 mM solution of 9 in ethanol and incubated at room temperature for 1-24 hours. Finally, twice-patterned surface-modified substrates were removed from the assembly bath, spin washed at 1000 rpm with ethanol and dried under a stream of nitrogen.

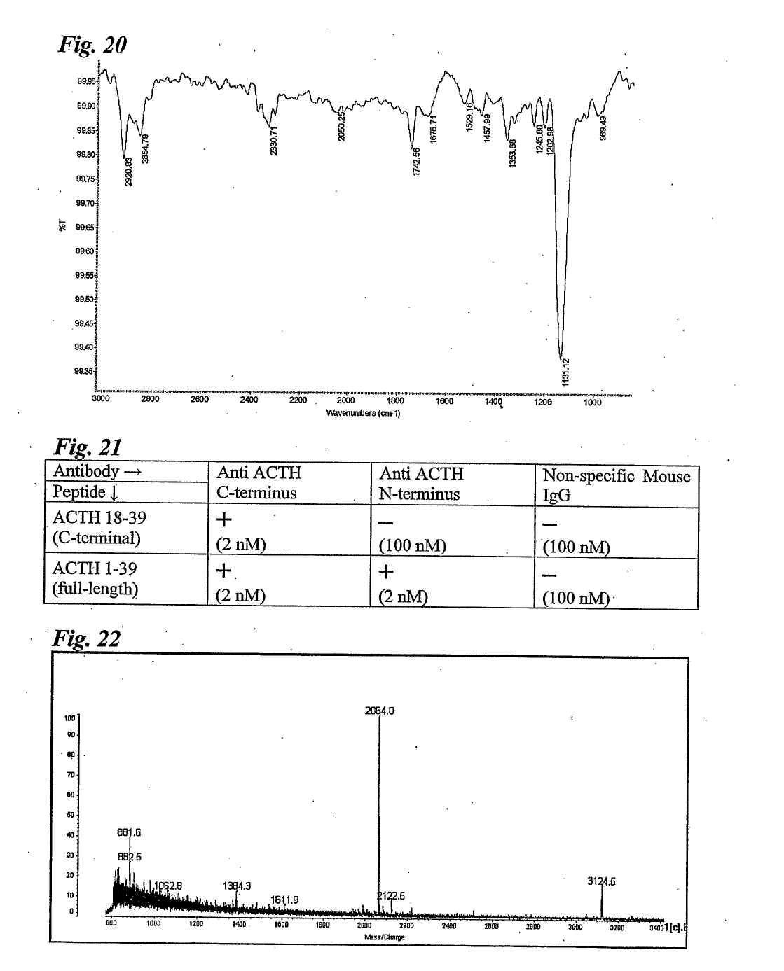

These twice-patterned surfaces were then incubated in 25 mM phosphate buffer, pH 8.0 for 15 minutes. The twice-patterned surfaces were then removed from the buffer solution and treated with an aqueous solution containing 1-[3-(dimethylamino)propyl]-3-ethylcarbodiimide hydrochloride (EDC; 0.4 M) and N-hydroxysuccinimide (NHS; 0.1M) for 60 minutes at room temperature. The substrates were subsequently washed with UHP water, followed by a wash with acetone and blown dry with a stream of nitrogen. The presence of the NHS-ester group was confirmed by grazing-angle FTIR (˜1742 cm⁻¹), as shown in FIG. 20.

### EXAMPLE XXIII

### Antibody Immobilization and Antigen Detection from Buffer and Diluted Serum Samples

An example of immobilizing antibodies to form a capture zone on the sample presentation device and using them to specifically capture and detect human peptide hormones from complex mixtures is illustrated below. Monoclonal antibodies directed against either the C-terminal (amino acids 18-39; Serotech, P/N MCA2000) or N-terminal (amino acids 1-24; Biodesign P/N E54057M) portions of human adrenocorticotropic hormone (ACTH), as well as a non-specific mouse immunoglobulin (IgG; Chemicon, P/N PP100) preparation, were dissolved to a final concentration of 0.5 mg/mL in a buffer containing 25 mM sodium phosphate, pH 8.0 and 0.1% Tween-20.

Ten μL of antibody solution was applied to the liquid retention zones of the X3 chip described in Example XXII and allowed to incubate for 60 minutes in a chamber held at 100% relative humidity (RH). The antibody solutions were washed off of the chip with excess Tris-buffered saline solution containing 0.05% Tween-20 (TBS/Tween) via a spray bottle. Excess wash solution was removed from the sites by gentle shaking, then 10 μL of a 50 mM ethanolamine (Sigma P/N E9508), pH 9.0, solution containing 0.1% Tween-20 was applied to the sites and allowed to incubate for 30 minutes at 100% RH (this action quenches any remaining activated NHS groups and prevents them from covalently binding other materials). Following incubation, the ethanolamine solution was washed off as above with TBS/Tween from a spray bottle. Each site was then treated with 5 μL of a 1% solution of Bovine Serum Albunin (BSA; Sigma P/N A3059) containing 0.5% Tween-20 to block any non-specific binding sites before addition of the analyte. The BSA solution was allowed to incubate on the chip for 30 minutes at 100% RH, followed by washing as described above.

5 μL aliquots of dilutions of ACTH peptides (either full length, amino acids 1-39, Bachem P/N H-4998; or C-terminal, amino acids 18-39, Bachem P/N H-1215) in either 10% rabbit serum or TBS/Tween were applied to the sites at peptide concentrations ranging from 2 nM to 100 nM. The peptides were incubated on the chip for 30 minutes at 100% RH, followed by washes with TBS/Tween, then UHP water, from a spray bottle. The chips were then dried under a stream of nitrogen gas. Two μL of a solution containing the matrix 2,5-dihydroxybenzoic acid (DHB; Bruker Daltonics P/N 203074), 0.5 mg/mL in acetonitrile/ethanol/0.1% TFA with 10 mM dibasic ammonium citrate (84:13:3) were applied to each site. The matrix solution spread to fill the 3 mm diameter of the site. Upon drying, the sample and matrix were concentrated into the analysis zone (AZ) of the X3 site with which it was associated. The chip was then secured in a holder in preparation for MALDI-TOF analysis.

MALDI-TOF analysis was performed in the positive ion mode on an Axima CFR (Kratos Analytical by Shimadzu Biotech, Manchester, UK) using a pulsed N₂ laser (337 nm), delayed extraction, and an acceleration voltage of 20 kV. The instrument was operated in reflectron mode using a semi-automated protocol producing, generally, 25-50 raster points/site, 20-50 shots per raster point. Data were collected and stored as an average of all raster points. Spectra were evaluated for the presence or absence of a distinct peak at the expected m/Z.

Both ACTH peptides diluted into 10% rabbit serum to a final concentration of 10 nM were readily detected by MALDI-TOF analysis on sites where monoclonal antibodies (mabs) directed against the C-terminus of ACTH had been immobilized. Spectra from sites with non-specific mouse IgG showed no capture of either ACTH peptide. No useful MALDI-TOF spectra could be obtained when 10% rabbit serum was applied to stainless steel plates, whether or not it contained additional ACTH peptide.

Application of a series of dilutions of each ACTH peptide in TBS/Tween buffer further illustrated the specificity and sensitivity of antigen capture and detection on these X3 chips. FIG. 21 shows that antibody/antigen pairing allow detection of the peptides at a 2 nM concentration, while the spectra of mismatched pairs showed no peptide binding from a high concentration (100 nM) solution. With the Anti ACTH C-terminus antibody capture zone, antigen concentration as low as 2 nM, for both ACTH 18-39 (C-terminal) and ACTH 1-39 (full-length) peptides, resulted in positive detection. With the Anti ACTH N-terminus antibody capture zone, when paired with ACTH 18-39 (C-terminal) peptide antigen, a negative result is obtained as expected, even when the concentration of the peptide is as high as 100 nM. With the Anti ACTH N-terminus antibody capture zone, when paired with ACTH 1-39 (full length) peptide antigen, a positive result was obtained, as expected, even when the concentrations is as low as 2 nM. With the control group, which utilized Non-specific Mouse IgG antibody on the capture zone, both ACTH 18-39 (C-terminal) peptide and ACTH 1-39 (full-length) peptide resulted in negative detection, as expected, at concentration as high as 100 μM. These data suggests that the device is highly sensitive and capable of detecting antigen at a low concentration (e.g. 2 μM). At the same time, the device has a high tolerance for false positive results, as indicated by negative detections even when the antigen concentration is very high (e.g. 100 nM).

These results illustrate the capability of this X3 surface to immobilize antibodies and use these antibodies to specifically and sensitively detect biomolecules from complex mixtures without the need for separate purification or concentration steps.

### Examples Utilizing Immobilized Metal Ions as Capturing Mechanism

In another variation, metal ions may be loaded onto the capture zone to provide a selective binding interface for capturing specific analytes. By loading the appropriate metal ion in the capture zone one may form an immobilized metal affinity chromatography (IMAC) surface in the capture zone.

In one variation, iron (i.e., Fe(III)) is loaded into the capture zone. In another variation, nickel (i.e., Ni(II)) is loaded into the capture zone. The metal loaded capture zone may be utilized in various applications for capturing selected analytes. In one example, a sample presentation device with Fe(III) loaded on the capture zone is utilized to capture phosphorylated peptides. In another example, a sample presentation device with Ni(II) loaded on the capture zone is utilized to capture His-tagged species. One of ordinary skill in the art having the benefit of this disclosure would appreciate that various metals may be selectively loaded onto the capture zone of a sample presentation device and then utilized to capture corresponding biologics, biochemicals, and/or chemicals for analysis and/or processing.

For example, a sample presentation device with a metal loaded capture zone may be particularly useful in proteomic research. Many proteomic studies specifically search for differences in post-translational modifications of proteins involved in disease pathways. One such modification is protein phosphorylation which can act as a switch to turn pathways on or off. A commonly used method to isolate phosphopeptides and proteins is immobilized metal affinity chromatography (IMAC). However, typical IMAC procedure may be cumbersome and/or expensive, and usually require multiple sample fluid transfer steps. Utilizing a sample presentation device with a metal-loaded capture surface the user may be able to minimize sample transfer and/or increase target molecule detection capability. In one example, an X3 surface containing a metal ion is utilized to chelate the phosphopeptide or protein, which then allows the non-bound material to be washed away. The chelated phosphorylated sample can then be eluted and co-crystallized with the matrix for MALDI-MS. This may decrease the overall sample-handling steps and therefore minimize the risk of sample loss and contamination.

In another variation, a sample presentation device is configured with another IMAC surface in the capture zone. In one example a prefabricated sample presentation device is converted to a Ni(II)-loaded surface. The resulting sample presentation device may be utilized to capture His-tag material from a sample solution. His-tags are often incorporated in recombinant proteins for ease of purification. After extensive washing of all the unbound material, the bound protein can be eluted by various means (e.g., excess imidazole or under acidic conditions, etc.) and isolated. Thus, the X3 IMAC surface may be utilized to capture His-tagged protein to the exclusion of proteins without the His-tag.

One of ordinary skill in the art having the benefit of this disclosure would appreciate that methods described herein may be utilized to prepare surfaces in a variety of devices for capturing selective species (e.g., phyosphotyrosine, His-tagged species, etc.) for further processing or analysis. In addition, it is also contemplated that other metals (e.g., Zn, Cu, etc.) may be loaded onto the capture zone to provide a selective binding interface for capturing specific analytes.

In one example, the following series of chemicals were synthesized to generate chemicals for the preparation of a prefabricated sample presentation device having capture zones configured for receiving and anchoring selective metal ions.

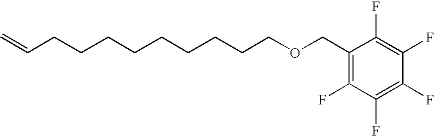

### 1,2,3,4,5-Pentafluoro-6-undec-10-enyloxymethyl-benzene (17)

A dry 200 mL round bottom flask was charged with 10-undecenyl alcohol (5.11 g, 30 mmol) under argon and 30 mL of dry tetrahydrofuran (THF) was added. The resulting solution was cooled to 0° C. and a solution of potassium t-butoxide (14.67 g, 200 mmol) in 60 mL of THF was added dropwise. The mixture was stirred under argon at 0° C. for 90 minutes. To the chilled, stirred solution was added 2,3,4,5,6-pentafluorobenzyl bromide (5.07 mL, 36 mmol) dropwise and the reaction was allowed to continue for 90 minutes at 0° C.. The reaction was quenched by the slow addition of 30 mL of water, and the total volume of the solution was reduced to 30-40 mL by rotary evaporation of the solvent. This was then diluted to 200 mL in ethyl acetate and then extracted with brine (1×200 mL) and water (2×200 mL). The organic layer was dried over magnesium sulfate, filtered and the solvent evaporated to give 17 as an oil. The residue was used ‘as is’ for the subsequent reaction.

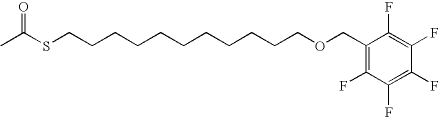

### Thioacetic acid 11-pentafluorophenylmethoxy-undecyl ester (18)

A dry, jacketed, 250 mL photoreaction vessel was charged with 17 (10.52 g, 30 mmol) and thiolacetic acid (10.72 mL, 150 mmol). These were dissolved into 150 mL of dry methanol and then 2,2′-azobis(2-methylpropionamide)dihydrochloride (814 mg, 3 mmol) was added. The UV lamp was activated and the mixture irradiated under argon with constant stirring for 4 hours. The reaction was continually cooled (water jacket) and the temperature maintained below 38° C. during the photo-reaction process. The reaction vessel was allowed to cool to room temperature and the solvent was evaporated to give a pale yellow oil. The oil was subjected to silica gel chromatography (41×300 mm, 1% ethyl acetate/hexane, increasing the ethyl acetate concentration by 1% for every two column volumes) and the fractions containing the desired product were collected, combined, and the solvent evaporated in vacuo to give 3.74 g (29%, two steps) of 18 as a colorless oil. ¹H NMR (400 MHz, CDCl₃): δ 4.46 (pt, 2H), 3.46 (t, 2H), 4.16 (t, J=6.8 Hz, 2H), 2.85 (t, J=6.0 Hz 2H), 2.31 (s, 3H), 1.72 (m, 2H), 1.56 (m, 2H), 1.24-1.36 (broad m, 14H).

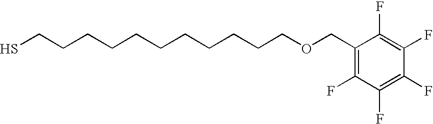

### 11-Pentafluorophenylmethoxy-undecane-1-thiol (19)

A 40 mL amber shell vial was fitted with a Teflon-lined silicon septum and charged with 18 (1.55 g, 3.6 mmol). This was dissolved into 10 mL of 4.9 N ethanolic hydrogen chloride and the resulting solution was warmed to 40° C. for 2.5 hours. The solvent was then evaporated in vacuo to afford a colorless oily residue. The residue was subjected to silica gel chromatography (41×450 mm, 5% ethyl acetate/hexane) and the fractions containing the desired product were then collected and combined. The solvent was evaporated in vacuo to afford 144 mg (10%) of 19 as a colorless oil. ¹H NMR (400 MHz, CDCl₃): δ 4.46 (pt, 2H), 4.17 (t, J=6.4 Hz, 2H), 2.51(dd, J=7.6, 14.6 Hz, 2H), 1.74 (m, 2H), 1.58 (m, 2H), 1.34 (t, 1H), 1.21-1.30 (broad m, 14H).

### EXAMPLE XXIV

### Preparation of Patterned Sample Presentation Devices (X3, C15-CO₂H Example)

Twenty-four surface-modified substrates were prepared as described in example XIII, mounted in a custom alignment jig and covered with a pin-registered etched stainless steel shadow mask (0.002 inch) having features corresponding in size and shape to the liquid retention zone. The jig was placed on the moving belt of an air-cooled ultraviolet curing system fitted with a low-pressure mercury light source rated at 120 W/cm² and passed under the light source 45 to 75 times over the course of one hour. Following UV exposure, the substrates were removed from the jig, spin washed at 1000 rpm with ethanol and dried under a stream of nitrogen. The exposed substrates were placed in a mixed solution containing 5% of 16-mercaptohexadecanoic acid and 95% of 19 (0.1 mM total thiol concentration) in ethanol and incubated at room temperature for a period ranging from 1 to 24 hours. Patterned surface-modified substrates were removed from the assembly bath, spin washed at 2400 rpm with ethanol and dried under a stream of nitrogen.

Patterned surface-modified substrates were mounted in a custom alignment jig and covered with a second pin-registered etched stainless steel shadow mask having features corresponding in size and shape to the analysis zone. The jig was placed on the moving belt of the ultraviolet curing system and passed under the light source 45 to 75 times over the course of one hour. Following UV exposure, the substrates were removed from the jig, spin washed at 1000 rpm with ethanol and dried under a stream of nitrogen. The exposed substrates were placed in a 0.1 mM solution of 9 in ethanol and incubated at room temperature for 1-24 hours. Finally, twice-patterned surface-modified substrates were removed from the assembly bath, spin washed at 1000 rpm with ethanol and dried under a stream of nitrogen.

### EXAMPLE XXV

### Preparation of NHS Chip and Subsequent Attachment of the NTA (Nitrilotriacetic Acid) Ligand

An X3 surface with an acidic group as described in Example XXIV was washed with a 10% solution of ammonia for 5 minutes and then rinsed with water. The surface was then dried with a stream of nitrogen. Into each well, 20 μL of 25 mM sodium phosphate (Sigma S9763) pH 8 containing 0.1% (v/v) octyl-β-glucoside (OBG, Pierce 28310) was applied and left to incubate for 10 minutes. The surfaces are then washed twice with 10 μL of 0.1% OBG (v/v) via pipet. The acidic groups are then reacted with NHS by applying 10 μL of a solution of 50 mM NHS (Pierce 24500) and 200 mM EDC (Pierce 22981) in 0.1% OBG and leaving it to incubate for 20 min. The surface is then washed twice with 10 μL of 0.1% OBG (v/v) via pipet. The next step involves reacting the NHS ester formed on the the surface with the chelating ligand. 10 μL of 20 mM AB-NTA (Dojindo A296) in 25 mM sodium phosphate buffer pH 8 containing 0.1% OBG is applied to the surface and left incubating for 30 min. The surfaces are then washed twice with 10 μL of 0.1% OBG (v/v) via pipet.

### EXAMPLE XXVI

### Loading Fe(III) onto the NTA Surface and Subsequent Sample Application

The next step is to introduce the buffer in which metal chelation will take place. The surface is therefore washed twice with 10 μL of 100 mM AcOH (acetic acid) in 0.1% OBG. The metal is then loaded by applying 10 μL of a 1 mM FeCl₃ (Sigma F-1513) solution in 1 mM AcOH ˜pH 3 containing 0.1% OBG and leaving it to incubate for 10 min. It is important to make the FeCl₃ solution up fresh because it tends to oxidize over time. After the 10 min of metal loading, the surface is washed twice with 10 μL of 100 mM AcOH in 0.1% OBG and then once with 10 μL of 100 mM AcOH in 0.1% OBG containing 1 M Urea (Stratagene 300191). The sample is then applied in the same solution and left to incubate for 20 min. The surface is subsequently washed once with 10 μL of the sample buffer and then twice with 10 μL of 100 mM AcOH. The surface is then allowed to air dry. The sample is then pre-eluted from the X3 surface and dried to the center by applying 2 μL of a 1:1 ACN(acetonitrile):0.1% phosphoric acid solution to each well. This releases the phosphopeptides from the Fe(III)-NTA surface into the solution which is then concentrated into the center. Once the wells are dried, 2 μL of matrix is applied to each well. The matrix formulation is 0.5 mg/mL DHB in 90:10 ACN: ammonium citrate (5 mM). The formulation leads to a uniform pad of crystals throughout the well.

An example application utilizing the Fe(III) loaded sample presentation device to pick out phosphopeptides from an in-gel tryptic digest is described below. β-Casein from bovine milk (Sigma part no. C6905, SwissProt P02666) was dissolved into 18 MΩ water to give a stock solution. This stock was then used to prepare the protein for 1-D gel electrophoresis. Thus, Laemmli buffer (Bio-Rad part no. 161-0737) was prepared according to the manufacturer's protocol and used to dilute the protein stock prior to use. Gel electrophoresis was performed using a pre-cast 4-15% SDS PAGE gel in Tris•HCl buffer (Bio-Rad product no. 161-1176) at 110 V constant voltage. Upon completion of the run, the gel was washed thoroughly with water to remove most of the SDS. The gel was then fixed by placing it in a 10% MeOH, 7% AcOH solution for 20 min. The gel was then stained using Sypro Ruby (BioRad no. 170-3125) overnight and subsequently destained using 10% MeOH, 7% AcOH. The bands were viewed using a transilluminator in a dark room and cut out of the gel using a razor blade cleaned with ethanol after removing each slice. In-gel tryptic digestion was performed following a known procedure (Rapid Comm. in Mass Spec. 2001, 15, 1416-1421), however, the gel slice was not extracted with the TFA mixture at the end and a Speedvac evaporator was never used to dry either the gel or the gel extracts. Thus, the supernatant from the in-gel digestion (20 μL/slice) was used without further purification or concentration. The final concentration of the digested sample stock was approximately 25 fmol/μL and was used without further dilution.

The Fe(III)-NTA surface was prepared as described in the preceding protocol until the incubation with 10 μL of 100 mM AcOH in 0.1% OBG containing 1 M Urea. At this point, 5 μL of the in-gel digest supernatant was added to each well, leading to a total volume of 15 μL per well. This was allowed to incubate on the surface for 20 min, and the rest of the procedure is as described previously. There was no problem focusing the solution as would be expected if gel contaminants had bound to the surface.

MALDI-TOF analysis was performed in the positive ion mode on an Axima CFR (Kratos Analytical by Shimadzu Biotech, Manchester, UK) using a pulsed N2 laser (337 nm), delayed extraction, and an acceleration voltage of 20 kV. The instrument was operated in reflectron mode using a semi-automated protocol producing, generally, 25-50 raster points/site, 20-50 shots per raster point. Data were collected and stored as an average of all raster points.

As indicated in FIG. 22, the major peaks (besides matrix cluster at 880 m/z) are the two phosphopeptides (2064 m/z and 3124 m/z) expected from digestion of β-casein. Noticeably absent are the trypsin auto-digestion peaks.

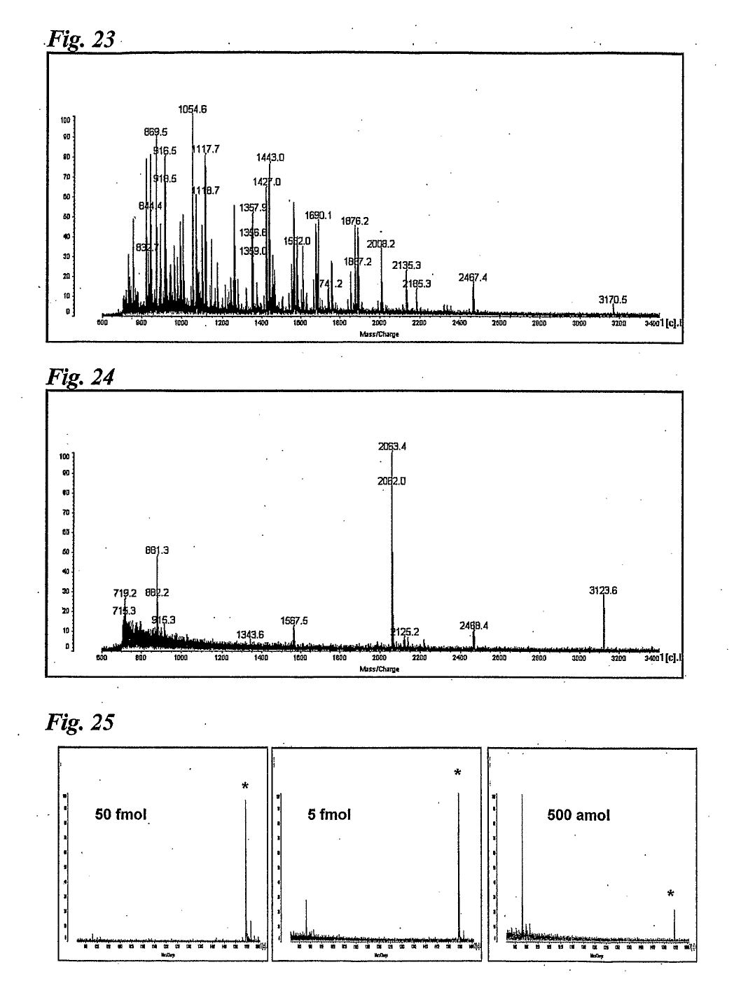

In another example application, the Fe(III)-loaded X3 IMAC surface is utilized to pick out phosphopeptides from a background tryptic digest. A mixture of 100 fmol phosphorylase b digest (Waters Corp. part no. 186002326, SwissProt P00489) and 5 fmol of β-casein digest were applied to the X3 IMAC surface using the unaltered protocol as described previously. Phosporylase b contains no phosphorylated amino acids and is used here to act as a sample contaminant. As seen in the FIG. 23 when the mixed protein digest sample solution was placed on a T3 surface, the background noise was sufficiently high such that it is difficult to identify the peaks for the phosphopeptides in the spectrum. The T3 surface does not have a binding surface that captures the phosphopetides and isolates the phosphopetides from undesired species. However, when the mixed protein digest sample solution was treated on the X3 IMAC surface, the signal to noise ratio improved significantly. As shown in FIG. 24, the X3 IMAC surface exhibits strong binding of the phosphopeptides (2064 m/z and 3124 m/z), which are not visible in the spectrum captured on the T3 (FIG. 23).

The Fe(III) loaded sample presentation device was then tested against a phosphorylated tyrosine peptide of various concentration to verify the device Ability in detecting low levels of phosphotyrosine-containing compound. Varying amounts of a phosphorylated tyrosine peptide, pp60^{C-SRC} carboxy-terminal phosphoregulatory peptide (Bachem, H-3258), were loaded onto a X3 IMAC charged with Fe(III) according to the protocol described previously. Good sensitivity was achieved using the Axima CFR as shown in FIG. 25.

### EXAMPLE XXVII

### Loading Ni(II) onto the NTA Surface and Subsequent Sample Application

In this example, the metal chelation took place in 0.1% OBG. The metal was loaded by applying 10 μL of a 10 mM NiSO₄.6H₂O (Sigma 227676) solution containing 0.1% OBG and leaving it to incubate for 10 min. After the 10 min of metal loading, the surface was washed once with 10 μL of 0.1% OBG and then twice with 10 μL of 10 mM HEPES (4-(2-hydroxyethyl)-1-piperazineethanesulfonic acid; Sigma H-4034) pH 7.4, 150 mM NaCl (Calbiochem 567441) with 0.1% OBG. The sample was then applied in the same solution in 5 μL and left to incubate for 20 min. The surface was subsequently washed three times with 10 μL of the sample buffer and then twice with 10 μL of water. The surface is then allowed to air dry. The sample was then pre-eluted from the Ni(II)-NTA surface and dried to the center by applying 2 μL of a 1:1 ACN:0.1% phosphoric acid solution to each well. These acidic conditions released the His-tagged protein from the IMAC surface into the solution which was then concentrated into the center. Once the wells were dried, 2 μL of matrix was applied to each well. The matrix formulation was 0.5 mg/mL DHB in 90:10 ACN:5 mM ammonium citrate. The formulation lead to a uniform pad of crystals throughout the well.

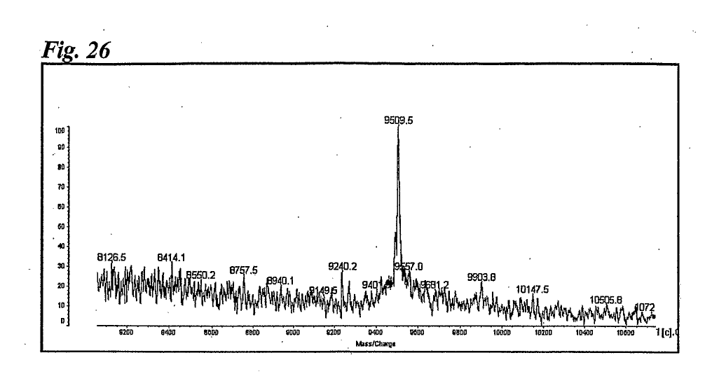

Ubiquitin from bovine erythrocytes (Sigma U-6253) and Ubiquitin, His-tag recombinant (Calbiochem 662060) were dissolved in 18 MΩ water and diluted to 20 fmol/μL in the HEPES buffer described above. Under the previously described protocol, 100 fmol of ubiquitin and the His-tag recombinant variant were loaded onto the chip. There was no binding of ubiquitin (m/z˜8500), and a strong signal visible for the His-tag recombinant ubiquitin (m/z˜9500, FIG. 26).

### Examples of Additional Presentation Device Configurations

As discussed above, the invention disclosed herein may be applied in various patterns, shapes or configurations for moving liquids around on a surface. In addition, one or more of the surfaces may be configured for capturing analytes and/or undesired species.

One variation that has been discussed earlier is the concentric arrangement which facilitates the movement of liquid toward a center region, and as a consequence, concentrates the liquid carrying the analyte. In one design, as shown in FIG. 1a, the sample presentation device comprises two concentric circles bounded by a boundary zone.

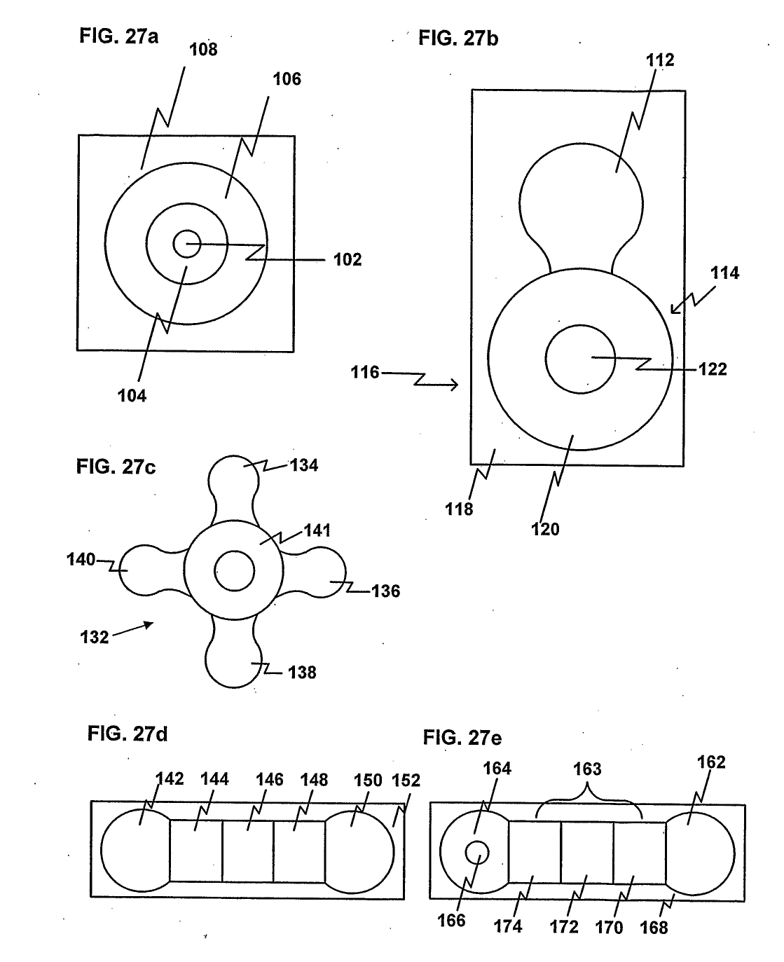

The sample presentation device may also be configured with three or more concentric circles for moving liquids toward a center region for analysis or processing. For example, the sample presentation device may comprise three concentric circles, as illustrated in FIG. 27a. Each region has a wettability value, and the wettability of adjacent region is different. The concentric rings may be configured such that the contact angle (CA1) of the surface in the center circle 102 is smaller than the contact angle (CA2) of the surface in the inner concentric ring 104, which is smaller than the contact angle (CA3) of the outer concentric ring 106, and which is smaller than the contact angle (CA4) at the boundary zone 108 (i.e., CA1<CA2<CA3<CA4).

In addition, one or more of the surface regions 102, 104, 106 may be modified to serve as binding regions. The various binding regions may be configured for capturing desired analyte, undesired species, or a combination thereof. For example, a first region may be configure for capturing analyte A, a second region may be configured for capturing analyte B, while a third region may be configured as the analysis zone. In another example, a first region is configured for capturing undesired specie C, a second region is configured for capturing undesired specie D, while a third region is configured as the analysis zone. In yet another example, a first region configured to capture an analyte, while a second region is configure to capture an undesired species, while a third region is configured as the analysis zone. For certain applications, it may be desirable to configure the analysis zone with a substantially non-binding surface. However, in other applications, the analysis zone may be configured with a binding surface.

In one example application, a first region is configured with a first antibody surface for capturing a first antigen, while a second region is configured with a second antibody surface for capturing a second antigen. A sample liquid containing both first and second antigens is introduced onto the sample presentation device. After an incubation period, which allows the antigens to bind to their respective antibody surfaces, the sample presentation device is washed to remove any residual sample liquid. The first antigen is then released from the first antibody surface and allowed to concentrate onto an analysis zone. Further processing and/or measurements may then be performed on the first antigen. After the first antigen has been analyzed, the operator may wash off the first antigen and continue to process the second antigen. The second antigen is released from the second antibody surface and allowed to concentrate onto the analysis zone. Chemical processing and/or measurements may then be performed on the second antigen.

The sample presentation device disclosed herein is not limited the concentric surface regions discussed above. Concentric designs are provided as examples for illustrating the various functionalities of the inventions. It is also contemplated that the sample presentation device may be configured with various non-circular patterns/shapes.

One variation is shown in FIG. 27b, where a “drop zone” 112 is added to the concentric circle design 114 similar to the one shown in FIG. 1a. In this configuration, the drop zone 112 is provided for receiving liquids delivered onto the sample presentation device 116. Preferably the drop zone 112 has a contact angle that is smaller than the contact angle of the boundary area 118 and larger than the contact angle of the liquid retention zone 120. A liquid droplet delivered to the drop zone 112 may travel towards the liquid retention zone 120 and eventually concentrates at the analysis zone 122. The liquid retention zone may also be modified to serve as the capture zone. In another variation, the drop zone is configured to supply sample liquid to a plurality of liquid retention zone and their corresponding analysis zone.

In another variation, the sample presentation device 132 is configured with two or more drop zones 134, 136, 138, 140 for receiving multiple droplets of liquids at different locations, which are then directed to flow toward the analysis zone after they have been released onto the surface of the sample presentation device 132. An example illustrating a four drop zone configuration is shown in FIG. 27c. The various droplets being delivered onto the sample presentation device may have the same chemical composition or different compositions. Furthermore, one or more of the zones on the sample presentation device may be configured with a binding surface. For example, the liquid retention zone 141 may be modified to capture the desired analyte.

The device shown in FIG. 27c may be particularly useful for introducing multiple liquids into a single region. For example, the device may be used for performing a series of chemical reactions by introducing reactants or catalyst into the drop zones one after the other. In a high throughput application, each reactant may be introduced with the same pipette by delivering the reactant onto a dedicated drop zone for each of the analysis zones. For example, a sample presentation chip may have 96 analysis zones, each of the analysis zones is surrounded by a liquid retentions zone, and each of the liquid retention zones may connect to four dedicated drop zones that are specific to each analysis zone. 96 different lead compounds may be tested on this chip at the same time by placing each of the 96 lead compounds in its own analysis zone on the sample presentation device. Four different reactants may be introduced one or more at a time. Since each of the four reactants may be introduce onto the 96 analysis zone through a dedicated drop zone for each analysis region, cross-contamination through the delivery pipette may be avoided. Furthermore, once the reaction has been completed, analysis of the end product may be conducted directly in the analysis (e.g., through mass-spectrometry, etc.).

In yet another variation, a sample presentation device, as shown in FIG. 27d, is configured with a liquid transport region having a contact angle gradient or transition-steps for transporting liquid from one region to the other on the surface of the sample presentation device. For example, the surface may comprise six regions (i.e., regions one through six) 142, 144, 146, 148, 150, 152 with corresponding contact angles CA21, CA22, CA23, CA24, CA25, and CA26. The regions may be configured such that CA21<CA22<CA23<CA24<CA25<CA26. Preferably, each of the contact regions is configured with a SAM.

In another example, the sample presentation device of FIG. 27e is configured with a drop zone 162, a liquid transport region 163, a liquid retention zone 164, and an analysis zone 166. Although in this particular example the liquid transport region comprises of only three regions 170, 172, 174 of varying wettability, one of ordinary skill in the art would appreciate that the liquid transport region may also comprise of four or more different wettability regions. For example, the liquid transport region may be configured with a continuous surface tension gradient from one end to the other such that there is a continuous transition in liquid contact angel on the surface of the liquid transport region. These continuous gradient or multi-step transition gradient may be use to move liquid reagents on a flat surface for various purposes. For example, two or more reagents may be transported from two different locations on a chip to a single location so that a chemical reaction may take place. In another variation, the surface tension gradient may be used to separate or filter various chemicals or fluids. For example, one or more of the liquid transport regions may be configured with a binding surface for capturing desire analytes and/or undesired species. In one variation, the liquid transport region is configured with various sections of antibody surfaces for capturing different antigens. The sample presentation device may then be utilized to filter the undesired antigens from a sample liquid deposited on the drop zone 162. In another variation, the liquid transport region 163 is adapted with one or more chromatographic surfaces/zones (e.g., ion-exchanges surface, reverse phase surface, etc.).

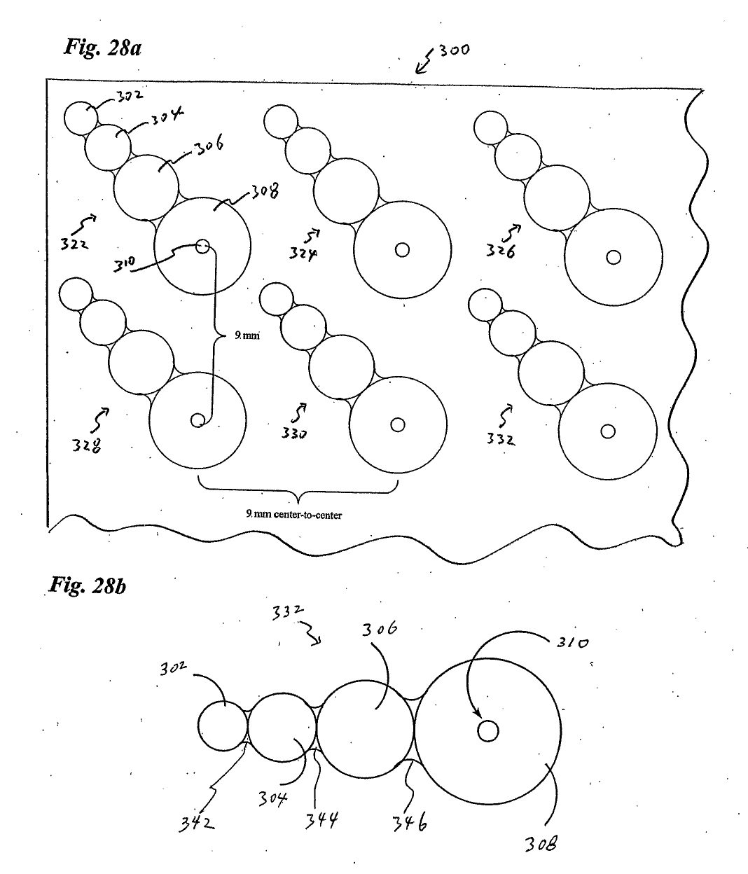

In one variation, the sample presentation device comprises a plurality of multiple juxtaposed zones of varying sizes, each with a specific purpose (e.g.; sample loading, sample purification/capture, liquid retention followed by concentration into the analysis zone and subsequent analysis, etc.). One example, as illustrated on FIG. 28a, comprises an 8×12 array of processing sites 322, 324, 326, 328, 330, 332 (only a portion of the 96 processing site are shown in FIG. 28a). FIG. 28b shows an individual processing site 322 from the sample presentation device 300 of FIG. 28a. The sample loading zone 302 may or may not contain functional chemistry for processing of the sample. The mechanism of transfer of liquid samples from one zone to another may vary. The sample loaded into the sample loading zone 302 may be moved mechanically (e.g., by pipetting, etc.) and placed into the next zone. The various zones 302, 304, 306, 308 described in the above sample presentation device may be connected by connection zones 342, 344, 346 and the wettability of which may permit liquid to flow from one zone to the other. In one variation, the different zones 302, 304, 306, 308, 310, 342, 344, 346 are each configured with different surface tension to promote liquid flow from one zone to the other. For example, each processing site 332 may be configured with a surface tension gradient to promote liquid flow from the loading zone 302 towards the analysis zone 310. In another variation, the wettability of each of the connection zones 342, 344, 346 may be changed in response to chemical or physical stimuli (e.g., UV radiation), such that the applied sample in the sample loading zone is transferred to the next zone 304 (e.g.; ion-exchange zone) when the exposure of a zone 342 between them to UV radiation results in a change of wettability and subsequent flow of liquid. Similar processes may then be used to transfer the sample from the ion-exchange zone 304 into a reversed-phase zone 306, and, ultimately, into the liquid retention zone 308. At this point the processed sample would be allowed to concentrate into the analysis zone 310. Alternately, the sample may be moved from zone to zone by the addition of a solvent which would result in an “overflow” into the next zone. This process may be repeated until the purified sample is in the liquid retention zone 308. Again, with a vast number of surfaces (having different wettability and/or analyte binding properties) and configurations thereof, sample presentation devices having a vast range of purification, concentration, isolation, and modification capabilities (vis-à-vis one or more analytes) can be created.

Ion exchange is one of the most frequently used chromatographic techniques for the separation and purification of proteins, polypeptides, nucleic acids, polynucleotides, and other charged biomolecules (“The right step at the right time.” BioTechnology, 4, 954-958 (1986), Bonnerjera, J., Oh, S., Hoare, M., Dunhill, P.). Thus, for various chemical analysis and/or synthesis applications, it may be advantageous to provide a functional zone on the sample presentation device with ion-exchange capability. The ion-exchange process may be implemented on the sample presentation device as the only active chemical process to take place prior to the analysis of the sample liquid in the analysis zone. In another variation, the ion-exchange may be implemented as one processing step within a series of two or more chemical processing steps to take place on the sample presentation device. The ion-exchange capability may also be implemented on a sample presentation device having one or more capture zones.

Separation in ion exchange chromatography depends upon the reversible adsorption of charged solute molecules to immobilized ion exchange groups of opposite charge. These ion exchange groups may be cationic or anionic, and are often categorized as ‘weak’ or ‘strong.’ Thus, the ion-exchange zone on the sample presentation device may be configured to comprise strong cationic functional groups (e.g.; sulfonate groups), weak cationic groups (e.g.; carboxylate groups), strong anionic groups (e.g.; quaternary amines), or weak anionic groups (e.g.; tertiary amines). The surface functionality may controlled through the assembly of various SAMs containing these functional groups. The surface wettability may be controlled through, for example, the use of 1°, 2°, 3°, or 4° compositions implemented in the assembly process.

As shown in the above example, reverse-phase chromatography is another chemical process that may be implemented in a functional zone on the sample presentation device. Molecules that possess some degree of hydrophobic character, such as proteins, peptides and nucleic acids, may be separated by reversed phase chromatography with excellent recovery and resolution. In addition, the use of ion pairing modifiers in the mobile phase may allow reversed phase chromatography of charged solutes, such as fully deprotected oligonucleotides and hydrophilic peptides. In one variation, the reversed-phase zone would contain n-alkyl hydrocarbon or aromatic groups, which can interact via hydrophobic interactions. The surface functionality may be controlled through the assembly of various SAMs containing these functional groups. The surface wettability may be controlled through, for example, the use of 1°, 2°, 3°, or 4° compositions implemented in the assembly process. One of ordinary skill in the art having the benefit of this disclosure would appreciate that the reverse phase zone may be implemented on the sample presentation device as the only active chemical process to take place prior to the analysis of the sample liquid in the analysis zone, or as one of a series of processes to take place on the sample presentation device. The reverse phase capability may also be implemented on a sample presentation device having one or more capture zones.

Ion-exchange zone and reverse phase zone are used herein as examples of possible functional regions/zones that may be implemented on a sample presentation device. One of ordinary skill in the art having the benefit of this disclosure would appreciate that various other chemical processes may be implemented in the functional regions by modifying the surface chemistry on a SAM surface within the functional zones.

Additionally, these sample presentation devices may be prepared in accordance with the Society for Biomolecular Screening (SBS) format. Thus, for example, the sites may be placed on 9 mm spacings in an 8×12 array, giving 96 individual sites for use. In the example shown in FIG. 28a, the sites are created on a 45° angle relative to the orthogonal positioning of the analysis zones to allow for the spacing. Versions compatible with a 384-site or 1536-site array could be created as well. One of ordinary skill in the art having the benefit of this disclosure would appreciate that a sample presentation device may be configured with various array configurations of sample processing sites.

Sample presentation device having one or more functional zones (e.g., ion-exchange zone, reverse-phase zone, capture zone, etc.) may be utilized to process various biological, biochemical and/or chemical samples. For example, crude biological samples (e.g.: serum, plasma, etc.), which contain a variety of species that could interfere with subsequent protein/peptide analysis, may be processed with a sample presentation device. In particular, sample “clean-up” has become a major area of research recently with the goal of removing the unwanted, interfering materials from the crude sample. Many of the current methodologies involve a series of off-surface chromatographic steps, each of which requires an instrument to control the chromatography and a column with which to process the crude sample. A sample presentation device having one or more functional zones to process the crude material may allow the operator to perform chromatographic processes on-surface and avoid the need for additional instrumentations and tools.

### Examples of Additional Presentation Device Implementation

In another variation, the presentation device comprises a surface having a capture zone and analysis zone. An optional boundary zone which surrounds the capture zone and the analysis zone may also be provided. The capture zone is configured such that it can be activated to capture an antigen. For example, the capture zone may be activated by covalently binding a chemical or biochemical (e.g., nucleotide, peptides, protein, etc.) onto the surface. The activated surface my then be utilized to capture analytes by non-covalent interactions with the analyte in the sample solution (e.g., a biological fluid, etc.). Once the desired analyte is bound into the capture zone, the residual species in the sample fluid may be removed from the surface of the presentation device (e.g., washing the surface of the sample presentation device).

Once the residual species has been removed, the desired analyte may be released from the capture zone and then transferred into the analysis zone for measurement. In one approach, the non-covalent bond linking the analyte to the activated surface may be disrupted through chemical (e.g., introduction of a chemical agent, etc.) or physical (e.g., UV irradiation, etc.) methods, thus, forcing the release of the analyte from the activated surface. The released analyte may then be directed to move into the analysis zone. For example, the released analyte may move towards the analysis zone due to surface tension deferential between the capture zone and the analysis zone. In one design variation, the capture zone has a larger surface area than the analysis zone, such that the analyte would concentrate onto the analysis zone. Once the analyte is in the analysis zone, various chemical analysis techniques may then be implemented to detect/measure the analyte. In one variation, the analysis zone comprises a substantially non-binding surface. In another variation, the analysis zone comprises binding surface for capturing the analyte for analysis. In another design variation, the binding characteristics of the analysis zone may be activated after the analyte has been captured in the capture zone and prior to the release of the analyte from the capture zone.

In another variation, the analyte is release from the capture zone by cleaving a covalent bond within the groups comprising the capture zone. The released analyte, which is attached to cleaved-end of the surface functional groups, may then be transported into the analysis zone for analysis or further processing.

One of ordinary skill in the art having the benefit of this disclosure would appreciate that the activation of the capture zone and the capturing of the analyte in the capture zone is not limited to the covalent and non-covalent bindings discussed above. Variations of the sample presentation device may implement utilizing ionic and other chemical binding characteristics, either alone or in combination with covalent/non-covalent binding, to activating the capture zone and capture antigen within the capture zone.

In another application, the sample presentation device is delivered and/or sold to a third party as a customizable device. The customizable device is configured with a modifiable capture zone, such that the third party can electively modify the capture zone to capture a specific analyte for analysis. In one variation, the capture zone on a sample presentation device is adapted with a surface that is capable of covalently binding an antibody. For example, the capture zone may be adapted with a SAM surface which comprises NHS ester groups. The third party may be the end user that modifies the sample presentation device, and then subsequently utilize the customized sample presentation device to analyze and/or process a particular analyte of interest. In another application, the third party may customize the sample presentation device for capturing a particular type of analyte, and then provide and/or sell the customized sample presentation device to another party, who may be the end user. In another example, the capture zone is adapted with a SAM surface comprising NTA ligands. A third party may then utilize the prefabricated NTA sample presentation devices to produce a sample presentation devices having different metal ions for capturing different bio-chemicals.

In yet another variation, the customizable sample presentation device is provided to a user in a kit along with other chemicals/solutions to allow the user to activate the capture zone with specific a chemical/biochemical to capture a desired antigen. In one variation, the kit comprises a sample presentation device having a capture zone that can be activated to anchor an antibody, and a chemical solution that can be utilized to activate and anchor the antibody. For example, the kit may comprise: (1) chips; (2) reagents; (3) buffers; (4) calibrants; and (5) tools. In another variation, the kit comprises a sample presentation device having a capture zone that can be activated for capturing phosphopeptides. For example, the kit may comprise: (1) chips; (2) reagents; (3) buffers; (4) calibrants; and (5) tools. An instruction on how to activate the capture zone by conjugating, loading, and/or anchoring the proper chemical/biochemical (e.g., nucleotide, peptides, protein, monoclonal antibody, iron ion, etc.) onto the capture zone may also be provided within the kit. In another variation, one or more of the activating agent to be conjugated, loaded, anchored and/or otherwise attached to the capture zone, may also be provided within the kit. For example, the kit may be provided with three types of monoclonal antibodies, so the user can customize the chip to capture one of the corresponding antigens.

### Example Applications Utilizing Various Detection/Measurement Mechanisms

As discussed earlier, the sample presentation device may be implemented for detection and measurement of various chemical, biochemical and/or biological samples with various detection and measurement apparatus. The sample presentation device may also be utilized to filter and/or concentrate chemical, biochemical and/or biological particles for further processing, and/or for running additional chemical reactions.

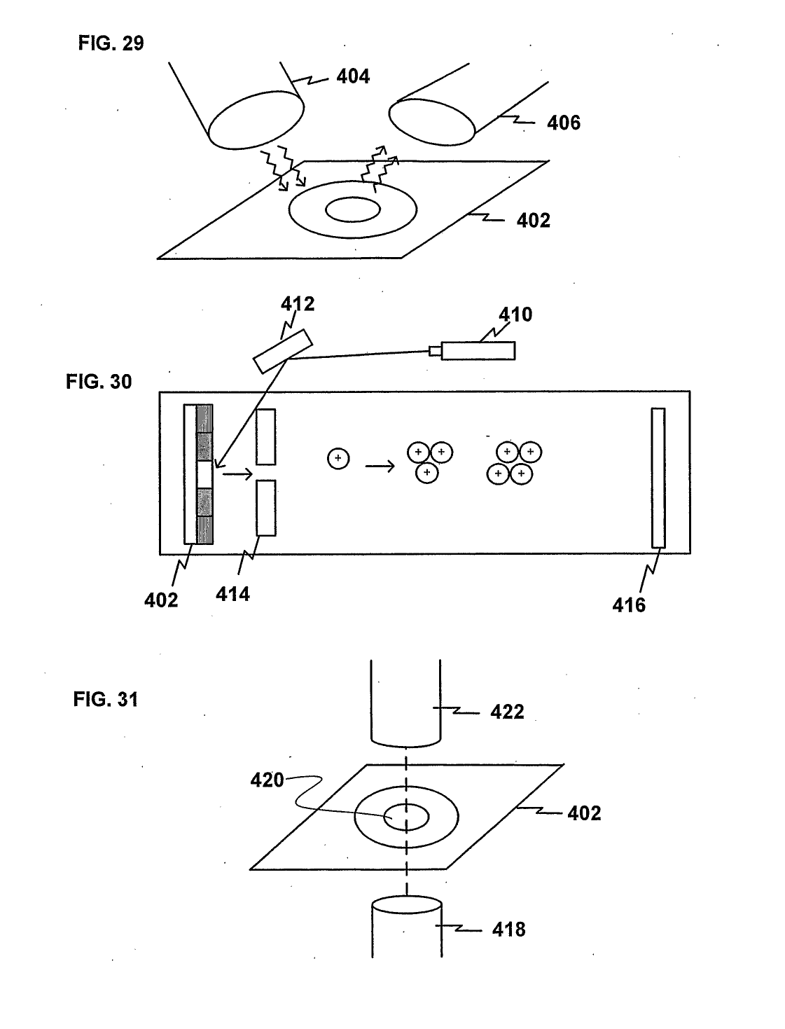

In one variation the sample presentation device 402 is utilized with a system for measurements based detection of reflected photons (e.g., optical spectroscopy, fluorescence detection, etc). The system may be configured with a photo-emitter 404 (e.g., UV, visible, or IR light source, etc.) and a photo-detector 408 (e.g., optical sensor, etc.), as shown in FIG. 29.

In another variation the presentation device is utilized with a system which ionizes the analytes on the sample presentation device 402 and directed the ionized particles toward the detector 416. For example the system may be a mass-spectrometer as shown in FIG. 30. A laser 410 directed by a mirror 412 may be utilized to excite the analytes on the surface of the sample presentation device 402, and the ionized particles are accelerated through the acceleration electrode 414 towards the detector 416.

In yet another variation, the sample presentation device may be configured such that photons may pass through the sample presentation device itself. For example, the substrate of the sample presentation device may 402 comprise a glass based layer with a thin gold layer sputtered on top, and a SAM layer deposited on the gold. The thin gold layer may allow transmission of photons. In one particular application, the sample presentation device 402 is utilized with an optical emitter 418 (e.g., IR light source, etc.) transilluminating light through the analysis zone 420 of the presentation device 402. An optical detector 422 (e.g., IR light detector, etc.) is positioned on the other side of the presentation device 402 to measure the amount of light that passes through the presentation device 402 and the analytes position in the analysis zone 420 (e.g., measuring IR absorption spectrum), as shown in FIG. 31. One of ordinary skill in the art having the benefit of this disclosure would appreciate that other detection and/or measurement apparatus may also be implemented with the sample presentation device described herein.

This invention has been described and specific examples of the invention have been portrayed. While the invention has been described in terms of particular variations and illustrative figures, those of ordinary skill in the art will recognize that the invention is not limited to the variations or figures described. In particular, the physical arrangement of the analysis zone, liquid retention zone, and boundary zone is not limited by the examples described above. In addition, where methods and steps described above indicate certain events occurring in certain order, those of ordinary skill in the art will recognize that the ordering of certain steps may be modified and that such modifications are in accordance with the variations of the invention. Additionally, certain of the steps may be performed concurrently in a parallel process when possible, as well as performed sequentially as described above. Therefore, to the extent there are variations of the invention, which are within the spirit of the disclosure or equivalent to the inventions found in the claims, it is the intent that this patent will cover those variations as well. Finally, all publications and patent applications cited in this specification are herein incorporated by reference in their entirety as if each individual publication or patent application were specifically and individually put forth herein.

We claim the following:

1. A sample presentation device comprising:
a substrate having a surface, wherein said surface comprises a first zone configured to capture an analyte and a second zone configured for analyzing said analyte, said first zone and said second zone being configured with different wettability to promote liquid flow from said first zone to said second zone.

2. The sample presentation device according to claim 1 wherein said surface further comprises a third zone, said third zone is configured to contain liquid within said first zone.

3. The sample presentation device according to claim 1 comprising a plurality of said first zone and a plurality of said second zone, wherein the plurality of said first zone is distributed on said surface as an array, each of said first zone is connected to a corresponding one of the plurality of said second zone, said surface further comprises a third zone adapted to separate said plurality of first zone.

4. The sample presentation device according to claim 1 wherein said substrate comprises a self-assembled monolayer.

5. The sample presentation device according to claim 1 wherein said first zone comprises an antibody.

6. The sample presentation device according to claim 1 wherein said first zone is configured for performing chromatography.

7. The sample presentation device according to claim 1 wherein said first zone comprises a immobilized Fe(III).

8. The sample presentation device according to claim 1 wherein said first zone comprises a immobilized Ni(II).

9. The sample presentation device according to claim 1 wherein said first zone comprises an immobilized metal affinity chromatography surface.

10. The sample presentation device according to claim 1 wherein said first zone is adapted to capture a protein, a peptide, or a nucleotide.

11. The sample presentation device according to claim 4 wherein said second zone is substantially non-binding.

12. The sample presentation device according to claim 11 wherein said first zone comprises an antibody for capturing an analyte.

13. The sample presentation device according to claim 11 wherein said first zone is configured for performing chromatography.

14. A method of analyzing an analyte comprising:
presenting said analyte on the sample presentation device as described in claim 9; and
detecting said analyte.

15. The method according to claim 14 wherein said detecting act comprises performing laser desorption ionization mass spectrometry on said analyte.

16. A method of analyzing an analyte comprising:
presenting said analyte on the sample presentation device as described in claim 11; and
measuring a chemical characteristic of said analyte.

17. A sample presentation device comprising:
a substrate having a surface for presenting an analyte for a analysis;
means for capturing said analyte; and
means for focusing said analyte for analysis.

18. The sample presentation device according to claim 17 wherein said substrate comprises a self-assembled monolayer.

19. The sample presentation device according to claim 17 wherein said surface comprises a plurality of zones having different wettability.

20. The sample presentation device according to claim 19 wherein at least one of said plurality of zones comprises an antibody for capturing said analyte.

21. The sample presentation device according to claim 19 wherein at least one of said plurality of zones comprises an immobilized metal affinity chromatography surface.

22. The sample presentation device according to claim 18 wherein said surface is adapted to focus said analyte onto a substantially non-binding region on said surface.

23. A customizable sample presentation device configured to detect an analyte in a sample comprising:
a substrate having a surface, wherein said surface comprises a first region adapted for modification by a user to capture an analyte, and a second region configured for receiving said captured analyte and present said capture analyte for analysis.

24. The customizable sample presentation device according to claim 23 wherein said first region and said second region being configured with different wettability to promote liquid flow from the first region to the second region.

25. The customizable sample presentation device according to claim 24 wherein said second region is substantially non-binding.

26. The customizable sample presentation device according to claim 25 wherein said substrate comprises a self-assembled monolayer.

27. The customizable sample presentation device according to claim 26 wherein said first region is adapted to receive an antibody.

28. The customizable sample presentation device according to claim 23 wherein said first region comprises a NHS ester group.

29. The customizable sample presentation device according to claim 23 wherein said first region is adapted to chelate a metal ion.

30. The customizable sample presentation device according to claim 23 wherein said first region comprises a NTA ligand.

31. A method of presenting an analyte comprising:
introducing a sample liquid onto a surface, wherein said sample liquid comprises an analyte and a specie;
capturing said analyte on a first region on said surface;
removing said specie from said surface;
releasing said analyte; and
focusing said analyte onto a second region on said surface.

32. The method according to claim 31 further comprising:
detecting said analyte.

33. The method according to claim 32 wherein detecting said analyte comprises one of a group consisting of mass spectrometry, surface plasmon resonance, fluorescence, atomic force microscopy, optical spectroscopy, bioluminescence, chemiluminescence, x-ray photoelectron spectroscopy, ellipsometry, electrochemical detection, phosphorescence, ultraviolet spectroscopy, visible spectroscopy, and infrared spectroscopy.

34. The method according to claim 31 wherein said capturing act further comprises capturing said analyte with an antibody.

35. The method according to claim 31 wherein said capturing act further comprises capturing said analyte with a chromatography surface.

36. The method according to claim 31 wherein said focusing act further comprises focusing said analyte due to variability in surface tension on said surface, wherein said surface comprises a self-assembled monolayer.

37. A method of purifying a sample liquid comprising;
introducing a sample liquid onto a surface, said sample liquid comprises an analyte;
performing chromatography on said sample liquid in a first region on said surface;
transferring said sample liquid onto a second region on said surface; and
detecting said analyte within said second region on said surface.

38. The method according to claim 37 wherein the performing chromatography act comprises performing ion-exchange chromatography.

39. The method according to claim 37 wherein the performing chromatography act comprises performing reverse phased chromatography.

40. The method according to claim 37 wherein said surface comprises a self-assembled monolayer.
